SEQUENCE LISTING (98 sequences)

SEQ ID NO: 1 - DNA, 19 bp, Artificial
  primer
gcagtggctacttgaagat

SEQ ID NO: 2 - DNA, 18 bp, Artificial
  primer
tctcggtgatggcactgg

SEQ ID NO: 3 - DNA, 19 bp, Artificial
  primer
cttactgataaggacgctc

SEQ ID NO: 4 - DNA, 20 bp, Artificial
  primer
ctcatagttcatcgtaggct

SEQ ID NO: 5 - DNA, 20 bp, Artificial
  primer
atgagattcttcctaatttg

SEQ ID NO: 6 - DNA, 19 bp, Artificial
  primer
ggtaatgaagtatttctgg

SEQ ID NO: 7 - DNA, 19 bp, Artificial
  primer
attattgccatgtacactt

SEQ ID NO: 8 - DNA, 21 bp, Artificial
  primer
gaatctttaagcctcacatag

SEQ ID NO: 9 - DNA, 19 bp, Artificial
  primer
gctgagccgactagtgatt

SEQ ID NO: 10 - DNA, 19 bp, Artificial
  primer
aagggaagcgaggacgtaa

SEQ ID NO: 11 - DNA, 18 bp, Artificial
  primer
cgctctcgtctgacattt

SEQ ID NO: 12 - DNA, 18 bp, Artificial
  primer
tcagacatttttaggtgg

SEQ ID NO: 13 - DNA, 20 bp, Artificial
  primer
cgaaggaaatgtcagatcaa

SEQ ID NO: 14 - DNA, 19 bp, Artificial
  primer
gactccatggagattgaag

SEQ ID NO: 15 - DNA, 19 bp, Artificial
  primer
ggagggacgggaagtagag

SEQ ID NO: 16 - DNA, 22 bp, Artificial
  primer
gtgaagaagcagtggatgtgcc

SEQ ID NO: 17 - DNA, 16 bp, Artificial
  primer
cgccagcaatggatac

SEQ ID NO: 18 - DNA, 19 bp, Artificial
  primer
tgcaaaagtggtttcgttc

SEQ ID NO: 19 - DNA, 18 bp, Artificial
  primer
aagacagcccggcgaaga

SEQ ID NO: 20 - DNA, 21 bp, Artificial
  primer
attccgggagaatgcgtctgc

SEQ ID NO: 21 - DNA, 19 bp, Artificial
  primer
tgcctaatgttttgtgatt

SEQ ID NO: 22 - DNA, 18 bp, Artificial
  primer
gacccaaaactacctgtc

SEQ ID NO: 23 - DNA, 22 bp, Artificial
  primer
ccctgtgtgtctctaaaccagc

SEQ ID NO: 24 - DNA, 15 bp, Artificial
  primer
ggtggcagggtcagt

SEQ ID NO: 25 - DNA, 18 bp, Artificial
  primer
gtggggctggtggccgtg

SEQ ID NO: 26 - DNA, 14 bp, Artificial
  primer
ggccantcgctgcc

SEQ ID NO: 27 - DNA, 16 bp, Artificial
  primer
tcaccaacccaagaaa

SEQ ID NO: 28 - DNA, 18 bp, Artificial
  primer
aaacaatatggtaatgaa

SEQ ID NO: 29 - DNA, 18 bp, Artificial
  primer
accaagaaagatggtgac

SEQ ID NO: 30 - DNA, 15 bp, Artificial
  primer
ggaagcgaggacgta

SEQ ID NO: 31 - DNA, 17 bp, Artificial
  primer
catttgtttggaatcgt

SEQ ID NO: 32 - DNA, 17 bp, Artificial
  primer
tgtcagatcaagacaca

SEQ ID NO: 33 - DNA, 18 bp, Artificial
  primer
cagaatcatcttcatcat

SEQ ID NO: 34 - DNA, 14 bp, Artificial
  primer
ggcccttcagtgga

SEQ ID NO: 35 - DNA, 20 bp, Artificial
  primer
tgataaaccagttagaaatc

SEQ ID NO: 36 - DNA, 14 bp, Artificial
  primer
gtaaaacccaactg

SEQ ID NO: 37 - DNA, 16 bp, Artificial
  PRIMER
gaaactggtggagctg

SEQ ID NO: 38 - DNA, 120 bp, Homo sapiens
aaggtcatccactgcagcggctacttgaagatccgccagtacagcctggacatgtcccccttcgacggctgctaccaaaacgtgggcctggtggccgtgggccactcgctgcctcccagc

SEQ ID NO: 39 - DNA, 120 bp, Homo sapiens
aaggtcatccactgcagcggctacttgaagatcaggcagtatatgctggacatgtccctgttcgacggctgctaccaaaacgtgggcctggtggccgtgggccactcgctgcctcccagc

SEQ ID NO: 40 - DNA, 300 bp, Homo sapiens
aaatccaactatggcagtttttgctagaacttcttactgataaggacgctcgagactgcatttcttgggttggtgatgaaggtgaatttaagctaaatcagcctgaactggttgcacagaaatggggacagcgtaaaaataagcctacgatgaactatgagaaactcagtcgtgcattaagatattattacgatggggacatgatttgtaaagttcaaggcaagagatttgtgtacaagtttgtctgtgacttgaagactcttattggatacagtgcagcggagttgaaccgtttggtca

SEQ ID NO: 41 - DNA, 300 bp, Homo sapiens
aaatccaactatgacagtttttgctagaacttcttactgataaggacgctcgagactgtatttcttgggttggtgataaaggtgaatttaagctaaatcagcctgaactggttgcacaaaaatggggacagcgtaaaaataagcctacgatgaactatgagaaactcagtcgtgcattaagatattattatgatggggacatgatttgtaaagttcaaggcaagagatttgtgtacaagtttgtctgtgacttgaagactcttactggatacagtgcagcggagttgaaccgtttggtca

SEQ ID NO: 42 - DNA, 240 bp, Homo sapiens
atgaaatagcctgcagaaaagctcatgagattcttcctaatttggtatgttgttctgcaaaaaaccttcgagatattgatgaagtctcatctctacttcgtacctccataatgagtaaacaatatggtaatgaagtatttctggccaagcttattgctcaggcatgcgtatctatttttcctgattccggccatttcaatgttgataacatcagagtttgtaaaattctgggctctggta

SEQ ID NO: 43 - DNA, 240 bp, Homo sapiens
atgaaatagcttgcagaaaagctcatgagattcttcctaatttggtacgttgttctgcaaaaaaccttcgagatgttgatgaagtctcatctctacttcgtacctctgtaatgtgtaaacaatatggtaatgaagtatttctggccaagcttattgttcaggcatgcgtatctatttttcctgattctggccatttcaaagttgataacatcagagtttgtaaaattctgggctgtggta

SEQ ID NO: 44 - DNA, 240 bp, Homo sapiens
gagtacaaagtggtggtgctgggctcgggcggggtaggcaaatccgccctgaccgtgcagttcgtgaccggcaccttcatcgagaaatacgaccccaccatcgaggacttctaccgcaaggagatcgaggtggattcgtcgccgtcggtgctggagatcctggacacggcgggcaccgagcagttcgcgtccatgcgggacctgtacatcaagaacggccagggcttcatcctcgtctac

SEQ ID NO: 45 - DNA, 240 bp, Homo sapiens
gagtacaaagtggtggtgctgggctcgggcggcgtgggcaagtccgcgctcaccgtgcagttcgtgacgggctccttcatcgagaagtacgacccgaccatcgaagacttttaccgcaaggagattgaggtggactcgtcgccgtcggtgctggagatcctggatacggcgggcaccgagcagttcgcgtccatgcgggacctgtacatcaagaacggccagggcttcatcctggtctac

SEQ ID NO: 46 - DNA, 250 bp, Homo sapiens
aagttacagaatacctatatgctaataaaatggctttccgatacccagaacctgaagacaaggccaaatatgttaaagaaagaacatggcggagtgaatatgattccctgctgccagatgtgtatgaatggccagaatctgcatcaagccctcctgtgataacagaatagaagcactcccctgataaatactttctgtgctccagggaaccccttttttcagacaagaagagataatgtcttcagtttta

SEQ ID NO: 47 - DNA, 251 bp, Homo sapiens
aagttacagaatacctgtatgctaataaaatggctttctcaatacccagaacctgaagacaaggcccaatatgttaaagaaagaatatgatggagtgaatatgattccctgctgccagatgtgtgtgagtggccagaatctgcatcaagccctcctgtgataacagaatagaagcattcccctgataaatactttctgtgctccagggaaccccttttttcagacaagaagagataatgtcctcagtttta

SEQ ID NO: 48 - DNA, 359 bp, Homo sapiens
ttagcagatttggatccagtggttgttacattctggtaccgagcccctgaactacttcttggagcaaggcattataccaaagctattgatatttgggctatagggtgtatatttgcagaactactaacgtcagaaccaatatttcactgtcgacaagaggacatcaaaactagtaatccttatcaccatgaccagctggacagaatattcaatgtaatgggatttcctgcagataaagattgggaagatataaaaaagatgcctgaacattcaacattaatgaaagatttcagaagaaatacgtataccaactgcagccttatcaagtatatggaaaaacataaagttaaaccagatag

SEQ ID NO: 49 - DNA, 360 bp, Homo sapiens
ttagcagatttggatccagtgattgttacattctggtactgagcccctgaattacttctttgagtaaggcattataccaaagctattgatagttgggcttatagggtgtatgtttgaagaactactaatgtcaaaaccaatatttcacggtcgacaagaggacatcagaactggtaatccttattaccatgactggctggacagaatactcaatgtaatgggatttcctgcaaataaagacggggaagatataaaaaagatgcctgaacattcaacattaatgaaagatttcagaagaaatatgtatactaactgcagccttatcaagtatatggaaaaacacaaagttaaaccagatag

SEQ ID NO: 50 - DNA, 150 bp, Homo sapiens
agctggaagattctttatgggtatcattaacagatcagcatgtccagctccccatggcaatgactgcagagaatcttactgtaaaacacaaaataagcagagaagaatgtgacaaatatgccctgcagtcacagcagagatggaaagctg

SEQ ID NO: 51 - DNA, 150 bp, Homo sapiens
agctggaagattctttatgggtatcattaacagatcagcatgtccagctccccatggcaatgactgcagagaatcttgctgtaaaacacaaaataagcagagaagaatgtgacaaatatgccctgcagtcacagcagagatggaaagctg

SEQ ID NO: 52 - DNA, 180 bp, Homo sapiens
  misc_feature: “y” at position 59 can be C ot T, and “R” at position 14, 78 and 119 can be A or G.
aaatccaactatgrcagtttttgctagaacttcttactgataaggacgctcgagactgyatttcttgggttggtgatraaggtgaatttaagctaaatcagcctgaactggttgcacaraaatggggacagcgtaaaaataagcctacgatgaactatgagaaactcagtcgtgcattaa

SEQ ID NO: 53 - DNA, 309 bp, Homo sapiens
  misc_feature: “y” at positions 86, 123, 150, 182, 232 and 263 can be C or T. “R” at positions 150 and 183 can be A or G. “W” at positions 189 and 275 can be A or T.
ttgctggagctctcctggaattagctgaagaacttctgaggattggcctgtcagtttcagaggtcatagaaggttatgaaatagcytgcagaaaagctcatgagattcttcctaatttggtaygttgttctgcaaaaaaccttcgagatrttgatgaagtctcatctctacttcgtacctcyrtaatgwgtaaacaatatggtaatgaagtatttctggccaagcttattgytcaggcatgcgtatctatttttcctgattcyggccatttcaawgttgataacatcagagagtttgtaaaattctggg

SEQ ID NO: 54 - DNA, 167 bp, Homo sapiens
aatttattgccatgtacacttacgagagttctgagcaaggagatttaacctttcagcaaggggatgtgattttggttaccaagaaagatggtgactggtggacaggaacagtgggcgacaaggccggagtcttcccttctaactatgtgaggcttaaagattcagag

SEQ ID NO: 55 - DNA, 167 bp, Homo sapiens
aatttattgccatgtacacttaccagagttctgagcaaggagatttaacctttcagcaaggagatgtgattttggttaccaagaaagatggtgaccggtggacaggaacagtgggcgacaaggccggagtcttcccttctaactatgtgaggcttaaagattcagag

SEQ ID NO: 56 - DNA, 167 bp, Homo sapiens
  misc_feature: “s” at position 24 can be G or C. “r” at position 62 can be G or A. “y” at position 96 can be either T or C.
aatttattgccatgtacacttacsagagttctgagcaaggagatttaacctttcagcaaggrgatgtgattttggttaccaagaaagatggtgacyggtggacaggaacagtgggcgacaaggccggagtcttcccttctaactatgtgaggcttaaagattcagag

SEQ ID NO: 57 - DNA, 190 bp, Homo sapiens
  misc_feature: “r” at positions 24, 88 and 129 can be G or A. “y” at position 69 can be C or T.
aacaatggccaaatccaactatgrcagtttttgctagaacttcttactgataaggacgctcgagactgyatttcttgggttggtgatraaggtgaatttaagctaaatcagcctgaactggttgcacaraaatggggacagcgtaaaaataagcctacgatgaactatgagaaactcagtcgtgcattaa

SEQ ID NO: 58 - DNA, 300 bp, Homo sapiens
caaatccaactatggcagtttttgctagaacttcttactgataaggacgctcgagactgcatttcttgggttggtgatgaaggtgaatttaagctaaatcagcctgaactggttgcacagaaatggggacagcgtaaaaataagcctacgatgaactatgagaaactcagtcgtgcattaagatattattacgatggggacatgatttgtaaagttcaaggcaagagatttgtgtacaagtttgtctgtgacttgaagactcttattggatacagtgcagcggagttgaaccgtttggtc

SEQ ID NO: 59 - DNA, 300 bp, Homo sapiens
caaatccaactatgacagtttttgctagaacttcttactgataaggacgctcgagactgtatttcttgggttggtgataaaggtgaatttaagctaaatcagcctgaactggttgcacaaaaatggggacagcgtaaaaataagcctacgatgaactatgagaaactcagtcgtgcattaagatattattatgatggggacatgatttgtaaagttcaaggcaagagatttgtgtacaagtttgtctgtgacttgaagactcttactggatacagtgcagcggagttgaaccgtttggtc

SEQ ID NO: 60 - DNA, 239 bp, Homo sapiens
atggctgagccgactagtgatttcgagactcctatcgggtggcatgcgtctcccgagctgactcccacgttagggcccctgagcgacactgccccgccgcgggacagctggatgttctgggcaatgctgccgccaccgccaccaccacttacgtcctcgcttcccgcagccgggtcaaagccttcctctgagtcgcagccccccatggaggccagtctctccccggggctccgcccccc

SEQ ID NO: 61 - DNA, 239 bp, Homo sapiens
atggctgagccgactagtgatttcgagactcctatcgggtggcctgcgtctcccgagctgactcccacgttagggcccctgaccgacactgccccgccgcgggacagctggatgttctgggcaattctgccgccaccgccaccaccgcttacgtcctcgcttcccgcagccgggtcaaagccttcctctgagtcgcagccccccatggaggccagtctctccccggggctccgcccccc

SEQ ID NO: 62 - DNA, 240 bp, Homo sapiens
  misc_feature: “m” at positions 26 and 44 can be either A or C. “r” at positions 56, 147, 207 and 226 can be either A or G. “s” at position 83 can be either G or C. “k” at position 126 can be either G or T.
atggctgagccgactagtgatttcgmgactcctatcgggtggcmtgcrtctcccgrgctgactcccacgttagggcccctgascgacactgccccgccgcgggacagctggatgttctgggcaatkctgccgccaccgccaccaccrcttacgtcctcgcttcccgcagccgggtcaaagccttcctctgagtcgcagccccccatrgaggcccagtctctccccrgggctccgccccyc

SEQ ID NO: 63 - DNA, 247 bp, Homo sapiens
  misc_feature: “r” at positions 29, 117, 133, 183 and 231 can be either A or G. “w” at positions 63, 192 and 226 can be either T or A.  “y” at positions 125, 158, 171 and 201 can be either T or C.  “m” at position 153 can be either C or A.
atggtactatttcttagtgttttaaattrgaacatatcttgcctcatgaagctttaaattatwattttcagtttctccccatgaagcgctctcgtctgacatttgtttggaatcgtrccactgcyggtctgcrccagatgtaccgtcctttcmaataygattttctgttgyaccttgtagtgrattctgcawatcatcttycccacctaaaaatgtctgaatgctwacacraataaattttataaca

SEQ ID NO: 64 - DNA, 247 bp, Homo sapiens
atggtactatttcttagtgttttaaattggaacatatcttgcctcatgaagctttaaattataattttcagtttctccccatgaagcgctctcgtctgacatttgtttggaatcgtgccactgctggtctgcgccagatgtaccgtcctttccaatacgattttctgttgcaccttgtagtggattctgcatatcatctttcccacctaaaaatgtctgaatgcttacacaaataaattttataaca

SEQ ID NO: 65 - DNA, 247 bp, Homo sapiens
atggtactatttcttagtgttttaaattagaacatatcttgcctcatgaagctttaaattattattttcagtttctccccatgaagcgctctcgtctgacatttgtttggaatcgtaccactgccggtctgcaccagatgtaccgtcctttcaaatatgattttctgttgtaccttgtagtgaattctgcaaatcatcttccccacctaaaaatgtctgaatgctaacacgaataaattatataaca

SEQ ID NO: 66 - DNA, 145 bp, Homo sapiens
  misc_feature: “r” at position 11  can be either G or A.  “y” at positions 84, 112 and 121 can be either T or C.  “w” at position 39 can either A or T.
aggaatttctrctgaaggaaatgtcagatcaagacacawgctgatgagtccaaaagctgatgttaaacttaagacttccagggygactgatgcttcaatctccatggagtcyttaaaaggyacaggagattcagtagatgaacag

SEQ ID NO: 67 - DNA, 180 bp, Homo sapiens
gaacaatcagtagtatacgttccaggaatttctgctgaaggaaatgtcagatcaagacacaagctgatgagtccaaaagctgatgttaaacttaagacttccagggtgactgatgcttcaatctccatggagtccttaaaaggcacaggagattcagtagatgaacagaattcctgcagg

SEQ ID NO: 68 - DNA, 180 bp, Homo sapiens
gaacaatccgtagtgtacgttcaaggaatttctactgaaggaaatgtcagatcaagacacatgctgatgagtccaaaagctgatgttaaacttaagacttccagggcgactgatgcttcaatctccatggagtctttaaaaggtgcaggagattcagtagatgaacagagttcccgcagg

SEQ ID NO: 69 - DNA, 191 bp, Homo sapiens
  misc_feature: “r” at positions 78, 89 and 101 can be either A or G.  “n” at positions 104, 107, 118, 122, 166 and 170  can A, or G, or T, or C,  or nothing.  “m” at position 109 can either C or A.  “s” at position 120 can be either G or C.
gtcaagaaatccctgaggagggacgggaagtagaggaattttcagaggacratgatgaagatgattctgatgactctraagcagaaaarcaatcacaaaarcancantmaagaggaantscnattctgatggcacatccactgcttcttcacagcagcaggctccnccgncagtctgttcctccttctcag

SEQ ID NO: 70 - DNA, 188 bp, Homo sapiens
gtcaagaaatccctgaggagggacgggaagtagaggaattttcagaggacaatgatgaagatgattctgatgactctaaagcagaaaaacaatcacaaaaacacaatcaagaggaactgcattctgatggcacatccactgcttcttcacagcagcaggctccccggcagtctgttcctccttctcag

SEQ ID NO: 71 - DNA, 188 bp, Homo sapiens
gtcaagaaatccctgaggagggacgggaagtagaggaattttcagaggacgatgatgaagatgattctgatgactctgaagcagaaaagcaatcacaaaagcagcataaagaggaatcccattctgatggcacatccactgcttcttcacagcagcaggctccgccgcagtctgttcctccttctcag

SEQ ID NO: 72 - DNA, 125 bp, Homo sapiens
caggcatggacgccagcaatggataccgggcccttcagtggaacgcaggctcaggtggactccctgagaacgaaaccacttttgcaagaatcttgcagcagcatggctatgcaaccggcctcata

SEQ ID NO: 73 - DNA, 124 bp, Homo sapiens
caggcatggacgccagcaatggatactgggcccttcagtggaatgcaggctcaggtggctccctgagaacgaaaccacttttgcaagaatcttgcagcaccgcgactatgcaactggcctcata

SEQ ID NO: 74 - DNA, 360 bp, Homo sapiens
gcccggtggaaaaagacagcccggcgaagacccaaagcccagcccaagacacctcaatcatgtcgagaaataacgcagatacaggcagagttcttgccttaccagagcacaagaagaagcgcaagggaaacttgccagccgagtccgttaagatcctccgcgactggatgtataagcatcggtttaaggcctacccttcagaagaagagaagcaaatgctgtcagagaagaccaatttgtctttgttgcagatttctaactggtttatcaatgctcgcagacgcattctcccggatatgcttcaacagcgtagaaacgaccccatcattggccacaaaacgggcaaagatgcccatgcca

SEQ ID NO: 75 - DNA, 360 bp, Homo sapiens
gcccggtggaaaaagacagcccggcgaagacccaaagcccagcccaagacacctcaatcatgtcgagaaataacgcagatacaggcagagttcttgccttaccagagcacaagaagaagcgcaagggaaacttgccagccgagtccgttaagatcctccgcgactggatgtataagcatcggtttaaggcctacccttcagaagaagagaagcaaatgctgtcagagaagaccaatttgtctttgttgcggatttctaactggtttatcaatgctcgcagacgcattctcccggatatgcttcaacagcgtagaaacgaccccatcattggccacaaaacgggcaaagatgcccatgcca

SEQ ID NO: 76 - DNA, 520 bp, Homo sapiens
  misc_feature: “k” at  4 and 179 can be either G or T.  “m” at 6, 21, 64, 93, 164 and 458 can be either C or A.  “n” at positions 16, 22, 246, 298, 322-325, 479-481, 485 and 499 can be any of A, C, G, and T. “s” at  27 and 410 can be either G or C.
cttkgmcttccttaanttccmnaactsaactccctttctaratcccattcattttctgcacctmccccataggyttgtttctctccattgytmttaaatgtaraaggccatacctggaatttwaaaaatatyattcctggtaatacagctcagtgtcattttcmtatttttaaaacatkctctayatgcctaatgttttgtgattcactttaacctgatggyttgcatttgctgtttttcactctnatgtcagarcagttgggttttacyccttagtttttatgcctgttragctttnctgtgcttttgacaggtagttttgggtcagttannnncagttttagtmttrtattccaagttgataactctwccatrtttcacatttctaaatttaacagagatgctrtagsttaaaaywtgttttgataagtaattacactggacctaggcaaaaccamtgaagaacaagtgttyncttnnntttnaccacatacayrtnatgttttgatcactgctgctt

SEQ ID NO: 77 - DNA, 531 bp, Homo sapiens
tccctttctagatcccattcattttctgcacctcccccataggcttgtttctctccattgctattaaatgtagaaggccatacctggaattttaaaaatattattcctggtaatacagctcagtgtcattttcctatttttaaaacatgctctacatgcctaatgttttgtgattcactttaacctgatggtttgcatttgctgtttttcactcttatgtcagagcagttgggttttaccccttagtttttatgcctgttaagctttactgtgcttttgacaggtagttttgggtcagttatattcagttttagtattgtattccaagttgataactcttccatgtttcacatttctaaatttaacagagatgctgtagcttaaaacatgttttgataagtaattacactggacctaggcaaaaccactgaagaacaagtgttcctttttaccacatacatattatgttttgatcactgctgcttgagccccgctattggtataattcagatcattttagcttgttgctga

SEQ ID NO: 78 - DNA, 532 bp, Homo sapiens
tccctttctaaatcccattcattttctgcacctaccccataggtttgtttctctccattgttcttaaatgtaaaaggccatacctggaatttaaaaaatatcattcctggtaatacagctcagtgtcattttcatatttttaaaacattctctatatgcctaatgttttgtgattcactttaacctgatggcttgcatttgctgtttttcactctatgtcagaacagttgggttttactccttagtttttatgcctgttgagctttctgtgcttttgacaggtagttttgggtcagttacagttttagtcttatattccaagttgataactctaccatatttcacatttctaaatttaacagagatgctataggttaaaatttgttttgataagtaattacactggacctaggcaaaaccaatgaagaacaagtgttttcttcccttttaccacatacacgtatgttttgatcactgctgcttgagtcctccaattggtataattcagatcacatttttagctagttgctga

SEQ ID NO: 79 - DNA, 210 bp, Homo sapiens
  misc_feature: “n” at positions 28 and 30 can be any of A, G, C, and T.  “s” at position 46 can be either G or C.  “r” at positions 71 and 180can be either G or A.  “k” at position 124 can be either G or T.  “y” at position 156 can be either C or T.
gacctggcgagcacaaagcctggcaccntngtctgctccattcacsatcaatgcacatcagagtgacatarcctgtgtgtctctaaaccagccaggcactgtagtggcctcagcctcccagaakggtacccttattcgcctctttgacacacaatycaaggagaaactggtggagctgcrccgaggcactgaccctgccaccctctactg

SEQ ID NO: 80 - DNA, 239 bp, Homo sapiens
tgggagtctgcaacttgtggacctggcgagcacaaagcctggcacctcgtctgctccattcacgatcaatgcacatcagagtgacatagcctgtgtgtctctaaaccagccaggcactgtagtggcctcagcctcccagaagggtacccttattcgcctctttgacacacaatccaaggagaaactggtggagctgcgccgaggcactgaccctgccaccctctactgcattaacttca

SEQ ID NO: 81 - DNA, 239 bp, Homo sapiens
tgggagtctgcaacttgtggacctggcgagcacaaagcctggcaccatgtctgctccattcaccatcaatgcacatcagagtgacataacctgtgtgtctctaaaccagccaggcactgtagtggcctcagcctcccagaatggtacccttattcgcctctttgacacacaattcaaggagaaactggtggagctgcaccgaggcactgaccctgccaccctctactgcattaacttca

SEQ ID NO: 82 - DNA, 167 bp, Homo sapiens; identical to SEQ ID NO: 54

SEQ ID NO: 83 - DNA, 167 bp, Homo sapiens
aatttattgccatgtacacttacgagagttctgagcaaggagatttaacctttcagcaaggggatgtgattttggttaccaagaaagatggtgaccggtggacaggaacagtgggcgacaaggccggagtcttcccttctaactatgtgaggcttaaagattcagag

SEQ ID NO: 84 - DNA, 7247 bp, Homo sapiens
gcgtccctcccagcggcgcgtgagcggcactgatttgtccctggggcggcagcgcggacccgcccggagatgaggcgtcgattagcaaggtaaaagtaacagaaccatggctcagtttccaacaccttttggtggcagcctggatatctgggccataactgtagaggaaagagcgaagcatgatcagcagttccatagtttaaagccaatatctggattcattactggtgatcaagctagaaactttttttttcaatctgggttacctcaacctgttttagcacagatatgggcactagctgacatgaataatgatggaagaatggatcaagtggagttttccatagctatgaaacttatcaaactgaagctacaaggatatcagctaccctctgcacttccccctgtcatgaaacagcaaccagttgctatttctagcgcaccaccatttggtatgggaggtatcgccagcatgccaccgcttacagctgttgctccagtgccaatgggatccattccagttgttggaatgtctccaaccctagtatcttctgttcccacagcagctgtgccccccctggctaacggggctccccctgttatacaacctctgcctgcatttgctcatcctgcagccacattgccaaagagttcttcctttagtagatctggtccagggtcacaactaaacactaaattacaaaaggcacagtcatttgatgtggccagtgtcccaccagtggcagagtgggctgttcctcagtcatcaaggctgaaatacaggcaattattcaatagtcatgacaaaactatgagtggacacttaacaggtccccaagcaagaactattcttatgcagtcaagtttaccacaggctcagctggcttcaatatggaatctttctgacattgatcaagatggaaaacttacagcagaggaatttatcctggcaatgcacctcattgatgtagctatgtctggccaaccactgccacctgtcctgcctccagaatacattccaccttcttttagaagagttcgatctggcagtggtatatctgtcataagctcaacatctgtagatcagaggctaccagaggaaccagttttagaagatgaacaacaacaattagaaaagaaattacctgtaacgtttgaagataaaaagcgggagaactttgaacgtggcaacctggaactggagaaacgaaggcaagctctcctggaacagcagcgcaaggagcaggagcgcctggcccagctggagcgggcggagcaggagaggaaggagcgtgagcgccaggagcaagagcgcaaaagacaactggaactggagaagcaactggaaaagcagcgggagctagaacggcagagagaggaggagaggaggaaagaaattgagaggcgagaggctgcaaaacgggaacttgaaaggcaacgacaacttgagtgggaacggaatcgaaggcaagaactactaaatcaaagaaacaaagaacaagaggacatagttgtactgaaagcaaagaaaaagactttggaatttgaattagaagctctaaatgataaaaagcatcaactagaagggaaacttcaagatatcagatgtcgattgaccacccaaaggcaagaaattgagagcacaaacaaatctagagagttgagaattgccgaaatcacccatctacagcaacaattacaggaatctcagcaaatgcttggaagacttattccagaaaaacagatactcaatgaccaattaaaacaagttcagcagaacagtttgcacagagattcacttgttacacttaaaagagccttagaagcaaaagaactagctcggcagcacctacgagaccaactggatgaagtggagaaagaaactagatcaaaactacaggagattgatattttcaataatcagctgaaggaactaagagaaatacacaataagcaacaactccagaagcaaaagtccatggaggctgaacgactgaaacagaaagaacaagaacgaaagatcatagaattagaaaaacaaaaagaagaagcccaaagacgagctcaggaaagggacaagcagtggctggagcatgtgcagcaggaggacgagcatcagagaccaagaaaactccacgaagaggaaaaactgaaaagggaggagagtgtcaaaaagaaggatggcgaggaaaaaggcaaacaggaagcacaagacaagctgggtcggcttttccatcaacaccaagaaccagctaagccagctgtccaggcaccctggtccactgcagaaaaaggtccacttaccatttctgcacaggaaaatgtaaaagtggtgtattaccgggcactgtacccctttgaatccagaagccatgatgaaatcactatccagccaggagacatagtcatggttaaaggggaatgggtggatgaaagccaaactggagaacccggctggcttggaggagaattaaaaggaaagacagggtggttccctgcaaactatgcagagaaaatcccagaaaatgaggttcccgctccagtgaaaccagtgactgattcaacatctgcccctgcccccaaactggccttgcgtgagacccccgcccctttggcagtaacctcttcagagccctccacgacccctaataactgggccgacttcagctccacgtggcccaccagcacgaatgagaaaccagaaacggataactgggatgcatgggcagcccagccctctctcaccgttccaagtgccggccagttaaggcagaggtccgcctttactccagccacggccactggctcctccccgtctcctgtgctaggccagggtgaaaaggtggaggggctacaagctcaagccctatatccttggagagccaaaaaagacaaccacttaaattttaacaaaaatgatgtcatcaccgtcctggaacagcaagacatgtggtggtttggagaagttcaaggtcagaagggttggttccccaagtcttacgtgaaactcatttcagggcccataaggaagtctacaagcatggattctggttcttcagagagtcctgctagtctaaagcgagtagcctctccagcagccaagccggtcgtttcgggagaagaatttattgccatgtacacttacgagagttctgagcaaggagatttaacctttcagcaaggggatgtgattttggttaccaagaaagatggtgactggtggacaggaacagtgggcgacaaggccggagtcttcccttctaactatgtgaggcttaaagattcagagggctctggaactgctgggaaaacagggagtttaggaaaaaaacctgaaattgcccaggttattgcctcatacaccgccaccggccccgagcagctcactctcgcccctggtcagctgattttgatccgaaaaaagaacccaggtggatggtgggaaggagagctgcaagcacgtgggaaaaagcgccagataggctggttcccagctaattatgtaaagcttctaaaccctgggacgagcaaaatcactccaacagagccacctaagtcaacagcattagcggcagtgtgccaggtgattgggatgtacgactacaccgcgcagaatgacgatgagctggccttcaacaagggccagatcatcaacgtcctcaacaaggaggaccctgactggtggaaaggagaagtcaatggacaagtggggctcttcccatccaattatgtgaagctgaccacagacatggacccaagccagcaatggtgttcagacttacatctcttggatatgttgaccccaactgaaagaaagcgacaaggatacatccacgagctcattgtcaccgaggagaactatgtgaatgacctgcagctggtcacagagatttttcaaaaacccctgatggagtctgagctgctgacagaaaaagaggttgctatgatttttgtgaactggaaggagctgattatgtgtaatatcaaactactaaaagcgctgagagtccgcaagaagatgtccggggagaagatgcctgtgaagatgattggagacatcctgagcgcacagctgccgcacatgcagccctacatccgcttctgcagccgccagctcaacggggctgccctgatccagcagaagacggacgaggccccagacttcaaggagttcgtcaaaagattggaaatggatcctcggtgtaaagggatgccactctctagttttatactgaagcctatgcaacgggtaacaagatacccactgatcattaaaaatatcctggaaaacacccctgaaaaccacccggaccacagccacttgaagcacgccctggagaaggcggaagagctctgttcccaggtgaacgaaggggtgcgggagaaggagaactctgaccggctggagtggatccaggcccacgtgcagtgtgaaggcctgtctgagcaacttgtgttcaattcagtgaccaattgcttggggccgcgcaaatttctgcacagtgggaagctctacaaggccaagaacaacaaggagctgtatggcttccttttcaacgacttcctcctgctgactcagatcacgaagcctttggggtcttctggcaccgacaaagtcttcagccccaaatcaaacctgcagtataaaatgtataaaacacctattttcctaaatgaggttctagtaaaattacccaccgacccttctggagacgagcccatcttccacatctcccacattgaccgcgtctatactctccgagcagaaagcataaatgaaaggactgcctgggtgcagaaaatcaaagctgcttctgaactttacatagagactgagaaaaagaagcgcgagaaagcgtacctggtccgttcccaaagggcaacaggcattggaaggttgatggtgaacgtggttgaaggcatcgagttgaaaccctgtcggtcacatggaaagagcaacccgtactgtgaggtgaccatgggttcccagtgccacatcaccaagacgatccaggacactctgaaccccaagtggaattccaactgccagttcttcatccgagacctggagcaggaagtcctctgcatcactgtgttcgagagggaccagttctcaccagatgattttttgggtcggacggagatccgtgtggcggacatcaagaaagaccagggctccaaaggtccagttacgaagtgtcttctgctgcacgaagtccccacgggagagattgtggtccgcttggacctgcagttgtttgatgagccgtaggcagcgggctcagggtgtgctcagcagggtcccagcccacggccacacatgctgtctggaaattgtattccttttctaagaaaccaccatttggtattcagtcacagggatatgggatggcaaagacaggcccctcaaagctcctaggaatcattctcgacaatcctccctgccccgaaacaatttcctgtttcatgaaacaaagctgtgttttcctttgtcctcactacaggtctcattatggcttctagggtcgctgaaatcccatagccctcaacagggtgcagctgggagtctagccccttcccgggcttgagggatgggtctggttactataaaatagatttataaatgcaatgtctatatttttggagaactcatgtaaccctcctgtttcttacatccaccagtccccaagtagacttcttggcctacaatgcccagtccttggtgtgagtttagaaacaattatgacggtcctgtcattgcttcagaatcccatctctcctgcagggaaatgctgcctagagctgatcactcggtgagacggtctgatcaggccctggcttagctctttgaagagctggtctatggaagtttccagcatgtgcaccgttatagccgttccttccccctctaggccttgtattaatatatgtcaatgaaaacacactggtgtattgttgcgtggattcagttctgattcccagcatgcttagaatatggtcacagaaagtcattatctagaaagtcacccctctgctggatcagatcactacaggtcactggaaaggcaactttacaatgttgggtcactgggtctcggttggcagccatgttggaaaaatctcttttggctcggaggcctgtgatatttcatagcagcagtcgttgctggtgacctgttctgtgcttgaatgtgctgaatcctgattgttgtaggacatttcaacagctctttttggtacgttccccaaaaagccatgtcctagatccccaaggcgtgaaaaggaaaaatatcaagctggaggttgggaaagaaaatgaaggcagtccattatgtggtgggtgaaagaccctaggaggatgcaagccccgcacatcccggggcaaagacctaagacacttttccaccctccaccaccccaacctcacataatatgcttgttgcaagagtcaggactttatgactatgtgccaagctgtttggtttgagttctttaatttttttttcccttaaatgccaggagatcatctggttagttagatagtaacttgatttgctaatgaaaagtgggggccgtgttttgtttgcatgttaatattctcataatcctagtttgttgtggtcatgaaatgccctttgcatgttctgttggtactggagtctagctttcctgtactagatggtgttctctttgattgtaggtccttagactttaattagggttatcaaagtgctttctaaatgatagcatcagcgttgtggcagagtacctcctttgctgggaactgaatgtgtagggttatcatttcccatgagagcccggtcatacttcaagcaatttttttaaaagtgtgtgttggaaaggacaacaaagtttacatttcatacttttaagaaatactttattatttatttattgaagatagtgtagaattttgtatcaagaacaacagacataagtattttttgaaacaagcaaatataccctgtagttagaaactttcaactgaacatgttagagaccaagtttaacttcaggcatgcatttgtttaccatttcccagcagaaaacatggttaaaatactttaagtttatattttttgatgttgttaagaaacttttaaattaaatctataaatagacatgcaactcatgctttcctatttctataaccaacaccgtttgtttagtgtatttatgaaagatatgctaccatggtagaaagaaaagtattcaatgtgtaaatt

SEQ ID NO: 85 - DNA, 4394 bp, Homo sapiens
gcctgggaggcgggaggggggttggggcttctcagcgccgattccgcgggaagggccctgggacctcacacttctagtcgcgggagctgcaggtcttacccggagagacgctgcacgtggagccctcgccgctgccgttctcagccggctctggagtgcgggcgggggcgacagggccgattccggagtgggactgatcctttgaaatactccagccatgactaaaagagaagcagaggagctgatagaaattgagattgatggaacagagaaagcagagtgcacagaagaaagcattgtagaacaaacctacgcgccagctgaatgtgtaagccaggccatagacatcaatgaaccaataggcaatttaaagaaactgctagaaccaagactacagtgttctttggatgctcatgaaatttgtctgcaagatatccagctggatccagaacgaagtttatttgaccaaggagtaaaaacagatggaactgtacagcttagtgtacaggtaatttcttaccaaggaattgaaccaaagttaaacatccttgaaattgttaaacctgcggacactgttgaggttgttattgatccagatgcccaccatgctgaatcagaagcacatcttgttgaagaagctcaagtgataactcttgatggcacaaaacacatcacaaccatttcagatgaaacttcagaacaagtgacaagatgggctgctgcactggaaggctataggaaagaacaagaacgccttgggataccctatgatcccatacagtggtccacagaccaagtcctgcattgggtggtttgggtaatgaaggaattcagcatgaccgatatagacctcaccacactcaacatttcggggagagaattatgtagtctcaaccaagaagatttttttcagcgggttcctcggggagaaattctctggagtcatctggaacttctccgaaaatatgtattggcaagtcaagaacaacagatgaatgaaatagttacaattgatcaacctgtgcaaattattccagcatcagtgcaatctgctacacctactaccattaaagttataaatagtagtgcgaaagcagccaaagtacaaagagcgccgaggatttcaggagaagatagaagctcacctgggaacagaacaggaaacaatggccaaatccaactatggcagtttttgctagaacttcttactgataaggacgctcgagactgcatttcttgggttggtgatgaaggtgaatttaagctaaatcagcctgaactggttgcacagaaatggggacagcgtaaaaataagcctacgatgaactatgagaaactcagtcgtgcattaagatattattacgatggggacatgatttgtaaagttcaaggcaagagatttgtgtacaagtttgtctgtgacttgaagactcttattggatacagtgcagcggagttgaaccgtttggtcacagaatgtgaacagaagaaacttgcaaagatgcagctccatggaattgcccagccagtcacagcagtagctctggctactgcttctctgcaaacggaaaaggataattgagccccaggacattctgagactccaaagtctttcttaaaatgtttagagcaagtatagctcttacctttattactgaatttgaatcttcttttatttctaggctgtacagtctgatgcatgatttttttataaatatttcatactcttgtgaatttggatctttttactttgagcatatattttagaatatgtgtatgttaaaggatctccacaatgtctgcagtgtgaaggcaggttcattgtggaatagtttaacagtcaggaaggctaaactggtcagtattaatgtgtagccctaccaaaaatagccagtagtatctgaaaatgaaaaataaatgaagtatctctaggaaacagtctggcttaactatttttgaaaatataactgtttcccctctctgctgctttagatgttgctttacatagaaccagaaaatggaatttctcagctaaagcatgtgtgcctgtttcatctaatcaagcagagctaaaatgttcataccgaataaatttatattaataaattactaaactaagagtatcaggttatttatatatttgcaagcaaaggacagtaagaagttgactggcaaaagagcagtgctgaaggaggagatccaggtttaaatctggcttattaactcaagccaattttaaggattttctgtatagattactcatgtcagaccaagaatttaaattattttgagagaggcatttaattctaataaaccagctgttataaaaattataaaatgatctctgtttttcctgtcagagatttaaaaaactgaaaaggtatacctcaacccaaaaataaaggtttgttttggtttgttatggcttccttttttaaaaaattaccctgtagtgccagtttattatgcaaagcagcttatattcctttgtttctgataaaatgaagactttaaatcagtcagtagtactttacctttcaaggcattagtaaattacttgcaaatagttttaaaaggaaaatacgacctttgttataggcagtcttctctttaagacaatacttttccacttgttttccttttccatattatatatgtgtattcatatagctgtatacatattcagttgatcattttataaacatatgaaggcataaagatatacagaagaaaaattattaaacaactcattttaagattcaaattaactaattcctgcatatatgacattccttacataagcgaacactaaacaaaaatggctagaaatgtctttttctttcttttctctctttgttgtttaaggtattaagcacgaattattacatgagactggcagatagctattaatcctcttacagatttgagaaagttgattctcaaatatttatgcaccttctccttcattgttttctttaaatctgtcctcttaaaaagcttcttaagagctcagttaatgcttttgacttaactaggagaaaaaggcatgataatacaggcaagatggcattgttagcaattctggtagtggtttggaatgaatcctaagaggcagggatcttaaggacaaggaagagaagagagagagggagggatctttgatctctttctctggtaatcttaatgcataattttactaaaacatgttctcaattcattcatattattaagctcttcctgcagttgatatctgagcagagtaagatttgtatttccatttttacttttttgaaagagaatatatggacagattattagtacaatttgggcactgtggttttaagaatatctgagtaaaataacaatatgaaataataaacagaagctctaacgtcaggtaacaaatagacagcaagaaaggttttgcaccatcctcttacggcctagagagttgacaagttgcttgtagttttaaaaaaataataaagtatacccttctggtatatcatcaagagcttaagaatcttggctttcatatttaaaatgcttttggggagacatatattaaaattttagccaagatgatagacatgtctcaattatatatgtgtgtgtatgtttttaaagctaaaaacattacttttagatccctagaatgaaaatttttttctcatctatgcaattcccatatggtttttttttaaatcatattttattcattttctccctttagcaattttcattttatttctcataatttgaacagagacagttctcatacatgatcagatgctttttttttcttcttaccatcatttatgcatgacataggtaatgtgactaatttctccagttgattcaagaaactcattactttgcctcaaattatatgtaaaatatttgttttacttaggttacagttatcagaaaggtagtttttttcttctattaaaatataacattgtgaaagaaaataaaatttatgctattctttgctttgtttttataaatgaatttttcatagaatttacagtatattcaaaggaagaaagataaaattattggtcatcatttgtaccttagaagtacaagaatttaagtaaaagaaatgttcatttttgttttaaaatttgttttccatgtgaagtttttattgagccaactttcatacatatcttgctagcctaaagtctaaatatttgtgttggcatcagaaaaacaaatgaggcagaattgctatgtgtggttgatcttcagataaattgactgatcacagttatttttgtatcagtctatgttattaggaaaaattgtttagttgttttctcccctgattaatggtgatattcaagtatgatacaaaaagaattgtaccaccaaaaaaaaaaaaaaaa

SEQ ID NO: 86 - DNA, 3463 bp, Homo sapiens
atggctgagccgactagtgatttcgagactcctatcgggtggcatgcgtctcccgagctgactcccacgttagggcccctgagcgacactgccccgccgcgggacaggtggatgttctgggcaatgctgccgccaccgccaccaccacttacgtcctcgcttcccgcagccgggtcaaagccttcctctgagtcgcagccccccatggaggcccagtctctccccggggctccgccccccttcgacgcccagattcttcccggggcgcaaccccccttcgacgcccagtctccccttgattctcagcctcaacccagcggccagccttggaatttccatgcttccacatcgtggtattggagacagtcttctgataggtttcctcggcatcagaagtccttcaaccctgcagttaaaaattcttattatccacgaaagtatgatgcaaaattcacagacttcagcttacctcccagtagaaaacagaaaaaaaagaaaagaaaggaaccagtttttcactttttttgtgatacctgtgatcgtggttttaaaaatcaagaaaagtatgacaaacacatgtctgaacatacaaaatgccctgaattagattgctcttttactgcacacgagaagattgtccagttccattggagaaatatgcatgctcctggcatgaagaagatcaagttagacactccagaggaaattgcacggtggagggaagaaagaaggaaaaactatccaactctggccaatattgaaaggaagaagaagttaaaacttgaaaaggagaagagaggagcagtattgacaacaacacaatatggcaagatgaaggggatgtccagacattcacaaatggcaaagatcagaagtcctggcaagaatcacaaatggaaaaacgacaattctagacagagagcagtcactggatcaggcagtcacttgtgtgatttgaagctagaaggtccaccggaggcaaatgcagatcctcttggtgttttgataaacagtgattctgagtctgataaggaggagaaaccacaacattctgtgatacccaaggaagtgacaccagccctatgctcactaatgagtagctatggcagtctttcagggtcagagagtgagccagaagaaactcccatcaagactgaagcagacgttttggcagaaaaccaggttcttgatagcagtgctcctaagagtccaagtcaagatgttaaagcaactgttagaaatttttcagaagccaagagtgagaaccgaaagaaaagctttgaaaaaacaaaccctaagaggaaaaaagattatcacaactatcaaacgttattcgaaccaagaacacaccatccatatctcttggaaatgcttctagctccggacattcgacatgaaagaaatgtgattttgcagtgtgttcggtacatcattaaaaaagacttttttggactggatactaattctgcgaaaagtaaagatgtataggcatctggtgtttcagcatacataactgaagcatgtgaaacagtatcatcctcgttagtagaggaaaaccaaaacccttttttccgtcaaaattggatttgtaattaaattgtaagcctcgtaggatgtatgttggaattttaagtctttcctttggttctatgcaaataaaaaaataactgattttttaagactgtgtctgtattgttgggattgaatctagtatttgctgggagaattttttctttgtatttattttaatgtattgttctcatgtaagaatgactgatgttgtgttagttaagaattgaagataggtttagcagtaaagaagaaagcttttaaaaggattgattcagctaagcaaagttgggcagagaaatacagccattttgtttttaatgcagaaaaggaagatgttctgtagcaagggggaatattttaaaaataaaccagatcaaattaatacaatcagaaggtttcgaaatgtaaatattccttatttaagacatgtttaaattcacctactagcacgacttacatagctcaaatattgaatgtttaaaatattaatacagatggggcctctttatgtttagataaaattgaagtacttaattgaagctttttaaaaattgtaaagtaaatgaaagctattgagatctttttgtctcctataataccagggaatttgagcttgtgttctagtcattgtactagctgtagctattggtctgtccttttgacatacagctaaaagggactaaatttgtaaaaaattagtttgttatagttgaagattaacttttcctaacattgtgattattgaagttcatgaatcttgctgtcaaggaagaaaggtaagaaagctgatagctcctccatgttggtaaaatcctctccagaatcttggaacacctggcatgtgaccctagtgacgtcacagacctgagatgaagattcatgtttagccagtgttttccagccttgtacccaccatacagatctgtttattctgtttcaccctactcctccagtgagccccatattttgggaaattatctgccttatacattaactaattcaattcatgtaacactgttgagtgcttactctttgtacctctattgtgcctatattaaaggtatacaaataaataaggccatgtctgacttcaaggaactcagtttaattttgatatattcaaagatgtgattcccaaccaactcaggatgaagtaactagtgttacaactgagttgatattctaaaatataacccagtttgtacttttattactagttagcatacacattttatggcttatgggttaataaatgaattcatggactcctggactactttcattgatgaccatatctccagggatgttgttgatccccacactgccttaaggtatattatagaaacagttttattttccatttttcttgtttcctgataataaatgtatttaggactgaaaatactcctgagtactcccctggctgtatgtctgacagtctttagctatggtgactattgtttatttttaatgggtatttcagattccaagtgtatttaaaatttctaaggagatataatatagcctgtatggtttctactttatggaattatatggtcaatatttgtaaatattctatgagttttgggtgggtagaggggtgctttgcctgttttgggtacaggtttttttggatttagcttgttaattgttcaaactttctgccttctacattcctatcttattgttcgtttaatcagtttctgaaatgtaagcattacatgactattggtgagttgtgccttttataactgaaatactttactttttctcatatcctctataattgacttctattttccttaatcaaaccagctctgggaaatttaatacatttatattaattgagattattaaaacatttggactattaaaaaaaaaaaaaaaaaaa

SEQ ID NO: 87 - DNA, 2505 bp, Homo sapiens
tttgggttagggagagtgctttcgtttgttttaaatgggagaaactggagcatgttgccaaggcagagagccagcagagaggggtgaatggaagaaggagcgagaagggggttactgacgaagccttatcctggaggagagaaggatggactccagagcccagctttggggactggccttgaataaaaggagggccactctacctcatcctggagggagcacgaacctaaaggcagacccagaagagctttttacaaaactagagaaaattgggaagggctcctttggagaggtgttcaaaggcattgacaatcggactcagaaagtggttgccataaagatcattgatctggaagaagctgaagatgagatagaggacattcaacaagaaatcacagtgctgagtcagtgtgacagtccatatgtaaccaaatattatggatcctatctgaaggatacaaaattatggataataatggaatatcttggtggaggctccgcactagatctattagaacctggcccattagatgaaacccagatcgctactatattaagagaaatactgaaaggactcgattatctccattcggagaagaaaatccacagagacattaaagcggccaacgtcctgctgtctgagcatggcgaggtgaagctggcggactttggcgtggctggccagctgacagacacccagatcaaaaggaacaccttcgtgggcaccccattctggatggcacccgaggtcatcaaacagtcggcctatgactcgaaggcagacatctggtccctgggcataacagctattgaacttgcaagaggggaaccacctcattccgagctgcaccccatgaaagttttattcctcattccaaagaacaacccaccgacgttggaaggaaactacagtaaacccctcaaggagtttgtggaggcctgtttgaataaggagccgagctttagacccactgctaaggagttattgaagcacaagtttatactacgcaatgcaaagaaaacttcctacttgaccgagctcatcgacaggtacaagagatggaaggccgagcagagccatgacgactcgagctccgaggattccgacgcggaaacagatggccaagcctcggggggcagtgattctggggactggatcttcacaatccgagaaaaagatcccaagaatctcgagaatggagctcttcagccatcggacttggacagaaataagatgaaagacatcccaaagaggcctttctctcagtgtttatctacaattatttctcctctgtttgcagagttgaaggagaagagccaggcgtgcggagggaacttggggtccattgaagagctgcgaggggccatctacctagcggaggaggcgtgccctggcatctccgacaccatggtggcccagctcgtgcagcggctccagagatactctctaagtggtggaggaacttcatcccactgaaattcctttggcatttggggttttgtttttccttttttccttcttcatcctcctccttttttaaaagtcaacgagagccttcgctgactccaccgaagaggtgcgccactgggagccaccccagcgccaggcgcccgtccagggacacacacagtcttcactgtgctgcagccagatgaagtctctcagatgggtggggagggtcagctccttccagcgatcattttattttattttattacttttgtttttaattttaaccatagtgcacatattccaggaaagtgtctttaaaaacaaaaacaaaccctgaaatgtatatttgggattatgataaggcaactaaagacatgaaacctcaggtatcctgctttaagttgataactccctctggagcttggagaatcgctctggtggatgggtgtacagatttgtatataatgtcatttttacggaaaccctttcggcgtgcataaggaatcactgtgtacaaactggccaagtgcttctgtagataacgtcagtggagtaaatattcgacaggccataaacttgagtctattgccttgcctttattacatgtacattttgaattctgtgaccagtgatttgggttttattttgtatttgcagggtttgtcattaataattaatgcccctctcttacagaacactcctatttgtacctcaacaaatgcaaattttccccgtttgccctacgccccttttggtacacctagaggttgatttcctttttcatcgatggtactatttcttagtgttttaaattggaacatatcttgcctcatgaagctttaaattataattttcagtttctccccatgaagcgctctcgtctgacatttgtttggaatcgtgccactgctggtctgcgccagatgtaccgtcctttccaatacgattttctgttgcaccttgtagtggattctgcatatcatctttcccacctaaaaatgtctgaatgcttacacaaataaattttataacacgcttaaaaaaaaaaaaaa

SEQ ID NO: 88 - DNA, 5520 bp, Homo sapiens
gggtttcaccgtgttggccaggatgatctcgattttctgacctcgtgatatgcccacctcggcctcccaaagtgctgggattacagacgtgagccactgcacctggcctatgttcaatgatattggtgctgtggagaaaaaataaagcaggaaagaggcagatagtagcgggaggtaggtaggttgtgggaagagagagttttgcaatttcaagtacctatatggttaggaagtgaaagcttcaatcagaaggtgacatgacatgacatatgacataagtcctaaagtgagccatgaagatatttgaggaaagagcttcctgggtagaagaaatcatacctgcagaagctataagttagttacaaattcccaggtcaaaagaaattatgaattataagaggtatacagaacagaagcagcatttggatgccggataatattattgtattttccttcatgttctcctgcgtagtttctgatgaagaacaatcagtagtatacgttccaggaatttctgctgaaggaaatgtcagatcaagacacaagctgatgagtccaaaagctgatgttaaacttaagacttccagggtgactgatgcttcaatctccatggagtccttaaaaggcacaggagattcagtagatgaacagaattcctgcaggggagaaataaagagtgcatcattgaaggatttatgtcttgaagacaaaagacgcattgcaaacttaattaaagaactggccagagtaagtgaggaaaaggaagtgacagaggaaagactgaaagctgagcaggagtcatttgagaagaagatcaggcagttggaagaacagaatgaactgatcatcaaagaaagggaagctcttcagctacagtatagagaatgccaagaacttctaagcctgtatcagaaatatttatcagaacaacaggagaagctcaccatgtctctctcagaacttggtgctgctagaatgcaggaacagcaggtatccagtagaaaaagcactctccagtgttcatctgtggaactggatggttcctacttgagcatagccagaccacagacctactatcaaaccaagcaaagacctaagtctgcagtccaggattcagcttcagaatcccttatagcatttaggaataattctttgaaaccagtaacccttcatcatcccaaagatgatctagataagataccatcagagaccacaacatgtaattgtgaatctccagggagaaaacctgcagtcccaacagagaaaatgccacaagaagaattgcacatgaaggaatgtccacatcttaagcctactcctagtcaatgctgtggtcatagacttgctgcagatcgtgttcatgacagccatcctacaaacatgacccctcaacatcctaagacacatccagaatcatgcagttattgtcggctttcttgggcatctctggtgcatggtgggggggcactgcaacccattgaaactttgaaaaagcagatctcagaagatagaaagcagcaactgatgcttcagaaaatggaactggaaattgaaaaggagcgccttcagcatctgctggcccagcaggagacaaagcttcttctaaaacagcagcagcttcaccagtctcgactggattacaattgtttattgaagtcaaactgtgatggctggctgcttggaacatcatcatctattaaaaagcaccaagaccccccaaacagtggagagaataggaaggagaggaagacagttgggtttcattcgcatatgaaagatgatgcccagtggtcatgtcaaaagaaagatacatgtagaccccaaagagggacagtgacaggagttagaaaagatgcgtctacatctcctatgccaacaggaagcctaaaggattttgtcaccacagcctcaccatcattacagcacaccacctcccggtatgagacatctttgttggatttggttcagtctctgagcccaaactctgcgcccaaacctcagcgctatccctccagagaagctggggcctggaatcatggtactttccgactcagtcctctaaaatcaacccggaagaagatggggatgcacagaacccctgaagagttggaggagaatcagattctggaagatatatttttcatttgacatattgcaaaattttcttaggaaatttgtgggtttcctcacatactgatctaggattttaaattatttcattgcaaagtaattgtgtctctcctttcacggggacttgtctcactagcatcctgttacgtattgaatatagaaatcattctaacaacccaggttattttcaatcagaccaggcattcgataacacactaagggggtaggaatggaagatggtatcttttatatgctaaacagattagaaaattaacataggatactttctgcgtggtggaaaccattgcatattcagcctcatttcaagagtgtttccttctcaaacttctgctagaaaatgctgactcacttttatatacaagaaaaacctttccactgaaaaatccctctgatttaaaagtaacccctttgaaacataagcagtttcagaaaggcagactctctcatctttctcatcctgttctctcactactacatttctgtaagtggtcctttaattctgaagttggagttggtgcccctgccatcacaaacaacacgagagccaattgtgagtcagtctcagtagccatcagcctggcagctgcccatcccataaccaaagagctcaatgggctggagggtgagacccagccaatccttgggagcaagcagcactaaatcacatcagggagtgattagtcctggggaaattcagtggaggcacaagcctgaagcactcttctaccaatgtttgggtgaccacccctccaatatcccagaatgaataagaacaatccctaggtatttttatttacccaagtgttcagcttattcaccccacccccccaccccccatcacacgtgctcctgtgaactttccatggttatgtctacggaaaatgccactagagagagagtgatgcaagctgctgcaaagctgatgggcttcctctggccctcccttttcctccacgaggattaaaggatacaagctgaccaggcctcacaggtgctctgctcgtggccccaaagaaccatctctacttgccagagtatgttccaccacccaagcagggtctccctactttttctcactggcctcgttttgacccagagaaagcctatggaagttatgcagtgcagacctcatcctgtctgctgtcttttctcccacagagctcttgagatgggtccttcagcttgcagtggtccctagtagcctctcaagctaatggggatgatgatcctggtttagccagagttcaaaactctccagtcattgaaagcaaggggagggtgtgttcccagatgtgtattacttgggaattttatttgatctggagggtgtggcttttttttctctcctttttaccaaccccaactaaactgggagtaagacttagtcagtgttggtagacaaagtcataccacccagggcagcctgcatgcagcgagtctgcgtggcttccctgaattactcttttaattaaaactagattatttcaatctagaaaagccttgttgagcagcctccttatccagatagatccaaagctcatgtctcttcaggctgagactggcgctgtcacctccacccagccttctttcacttggggttttttattctttggagtagaaaagaatggagcccactaatattatatttttcttctgaaataaatataaagtcaagactaactagttataatcattcccttaaaaagttggaaaatcattacagattaaaatttctttataaagttcacctctgagagtaaccaaatcggtttcatcccatatcaaaaagcctttggagtgtggagctttctgtggtatggcagaaatgggtggatggcaaactaagagccctcagccattttttcagttaaagttaggttgccagaactttcttttccttgccccctgtgtcatgactagcttaagtgtacttgactcccatagactactcccatgcccacagtcacccattccagactccagtgtccttaaaactctggagtgagaggatgccagaggatcaagaaaaggatcacagtttcttgaagaagcttgtttgcctttagaagaatcgtaaacagagtctaaactaaaggctttttgggggtcacagccacagagtggagttttattgcttctttgcctctccatgaatggccaatttggaaaagcagagatgggctttcagcagaatatcaaccaatacatttttcacatgcaaaactcatcaccagttgcctttttctaacttaatggacattttgttggtgttggtgcaagggcaataggatgtaaatttgtatataatctaatgtcttcattattaattgaatagtacatgttaacattttaatctatttaaatttactttgaaatatatacatacatatgaatgaaaggttgttacagagcctcaaagctgttgcagactatcccaagagaaaggatctggcacaaaggaatctgcctttcctccgtctcaagtctcccacccttgactggttgggatttgctcgtccagcctctagacacttcccaaagcaagactggacacatgccgagggcgttgcggatagtgcctcaccattgcccaccctgctgcccaactcctgtgagagaaaaaccaatcaatgtttacaaaatggaaaaggacacagcatgtcctttgaactccctaagtaaggcccacagttcttgatgcagagaaccaaagtgaatcatagaaaagcttttgctaacagtccgctttccaggaggcaaacttgtgtttatccaaacctgatccccatgatggggacttttctagagcaccccaaatgtcatggggagagagggactctttctgctccctcagagctactcttcacttgtccctgcttccggcaccaagttcataatagagatcttctggccagagaatcgggagaggagaggcctgatggggcagagctggacgaagtctgctaagagagatggaggctgtggcggactccttccaactcacgatttatcctcagctcagagtgtattttgaatatgagcaaatgtttattcaatcttcaagaagtatcaagtccatccaggcgcggtggcttacacctgtaatcccaggctttgggaggccgaggcagccggatcatgaggtcaggagatcgagaccatcctggctaacacggtgaaaccccatctctgtattttgtatttgtaaaaatacaaaaaattacctgagcgtggtggcacgcacctgtagtcccggctactcaggaggctgaggcaggagaatcacttgaacccgggaggcggaggttgcagtgagccaagatcatgccgctgcactccagcctgggcgacagagcaagactccatctc

SEQ ID NO: 89 - DNA, 2690 bp, Homo sapiens
agatggcggtagctgaggggttgaccgagagacccagttgaaggcctttacgaagtgaaagaggccgggagtcgccccctacccgcttctcgtagtcctgggagcacagcagaagtgtttttctttttttaatgaacaagtaaaccatacaaattgtcaacatgggacggagatctacatcatccaccaagagtggaaaatttatgaaccccacagaccaagcccgaaaggaagcccggaagagagaattaaagaagaacaaaaaacagcgcatgatggttcgagctgcagttttaaagatgaaggatccaaaacagataatccgagacatggagaaattggatgaaatggagtttaacccagtgcaacagccacaattaaatgagaaagtactgaaagacaagcgtaaaaagctgcgtgaaacctttgaacgtattctacgactctatgaaaaagagaatccagatatttacaaagaattgagaaagctagaagtagaatatgaacagaagagggctcaacttagccaatattttgatgctgtcaagaatgctcagcatgtggaagtggagagtattcctttgccagatatgccacatgctccttccaacattttgatccaggacattccacttcctggtgcccagccaccctctatcctaaagaaaacctcagcctatggacctccaactcgggcagtttctatccttcctcttcttggacatggtgttccacgtttgccccctggcagaaaacctcctggccctccccctggtccacctcctcctcaagtcgtgcagatgtatggccgtaaagtgggttttgccctagatcttccccctcgtaggcgagatgaagacatgttatatagtcctgaacttgcccagcgaggtcatgatgatgatgtttctagcaccagtgaagatgatggctatcctgaggacatggatcaagataagcatgatgacagtactgatgacagtgacaccgacaaatcagatggagaaagtgacggggatgaatttgtgcaccgtgataatggtgagagagacaacaatgaagaaaagaagtcaggtctgagtgtacggtttgcagatatgcctggaaaatcaaggaagaaaaagaagaacatgaaggaactgactcctcttcaagccatgatgcttcgtatggcaggtcaagaaatccctgaggagggacgggaagtagaggaattttcagaggacgatgatgaagatgattctgatgactctgaagcagaaaagcaatcacaaaagcagcataaagaggaatcccattctgatggcacatccactgcttcttcacagcagcaggctccgccgcagtctgttcctccttctcagatacaagcacctcccatgccaggaccaccacctcttggaccaccacctgctccaccattacggcctcctgggccacctacaggccttcctcctggtccacctccaggagctcctccattcctgagaccacctggaatgccaggactccgagggcccttaccccgacttttacctccaggaccaccaccaggccgaccccctggccctcccccaggtccacctccaggtctgcctcctggtccccctcctcgtggacccccaccaaggctacctccccctgcacctccaggtattcctccacctcgtcctggcatgatgcgcccacctttggtgcctccccttggacctgccccccctgggctgttcccaccagctcccttgccaaaccctggggttttaagtgccccacccaacttgattcagcgacccaaggcggatgatacaagtgcagccaccattgagaagaaagccacagcaaccatcagtgccaagccacagatcactaatcccaaggcagagattactcgatttgtgcccactgcactgagagtacgtcgggagaataaaggggctactgctgctccccaaagaaagtcagaggatgattctgctgtgcctcttgccaaagcagcacccaaatctggtccttctgttcctgtctcagtacaaactaaggatgatgtctatgaggctttcatgaaagagatggaagggctactgtgacagcttttgatgccagaaaaggcttctgttcacaacagtggcccatggagaaagaggctcttattaaacttagatgaaagagctgcttccattgtcagggtattttctaatttcagttcaaggaatatcctaaaatttagccttgttcagaatttactgcacataaaaaagggtatttcatccagaatagatcagttattgaagcagtgctgctaacatccattccctttcataccaccattttcaccctgtttcttcccctcctccagttctttggaaatttgtgatcgggggatcttagttgcttatttgttttgactcttgtgtgctgtgggcactggagtagagatttctggagaaaaaaaaacagtttatttcatcttgccttttgtgtttgagttatttttaatattttcctgtaaatattttgtaatattttacttgtaatgaaatggatcacaatgtcatttcctaatacaaggcaggatatgtgggaagaatatgtacaattatttgattaaaattatttcccactgacctaaactttcagtgatttgtgggaaaaataaataaatgttctacaccaaaaaaaaaaaaaaaaaa

SEQ ID NO: 90 - DNA, 2167 bp, Homo sapiens
atgcgatccgccgcgcggaggggacgcgccgcgcccgccgccagggactctttgccggtgctactgtttttatgcttgcttctgaagacgtgtgaacctaaaactgcaaatgcctttaaaccaaatatcctactgatcatggcggatgatctaggcactggggatctcggttgctatgggaacaatacactgagaacgccgaatattgaccagcttgcagaggaaggtgtgaggctcactcagcacctggcggccgccccgctctgcaccccaagccgagctgcattcctcacagggagacattccttcagatcaggcatggacgccagcaatggataccgggcccttcagtggaacgcaggctcaggtggactccctgagaacgaaaccacttttgcaagaatcttgcagcagcatggctatgcaaccggcctcataggaaaatggcaccagggtgtgaattgtgcatcccgcggggatcactgccaccaccccctgaaccacggatttgactatttctacggcatgcccttcacgctcacaaacgactgtgacccaggcaggccccccgaagtggacgccgccctgagggcgcagctctggggttacacccagttcctggcgctggggattctcaccctggctgccggccagacctgcggtttcttctgtgtctccgcgagagcagtcaccggcatggccggcgtgggctgcctgtttttcatctcttggtactcctccttcgggtttgtgcgacgctggaactgtatcctgatgagaaaccatgacgtcacggagcaacccatggttctggagaaaacagcgagtcttatgctaaaggaagctgtttcctatattgaaagacacaagcatgggccatttctcctcttcctttctttgctgcatgtgcacattccccttgtgaccacgagtgcattcctggggaaaagtcagcatggcttatatggtgataatgtggaggagatggactggctcatagccagtgacttcatgtcatcatcagaagttaccgaaagtgaagcgataaagttaatgttcaggacaatgcagagacgctgtcttccttctatggccttcaagaaaccctggagaggaccagtgaggctgcagattcttaaaagagcatagacattaaaattcttgcagaatctgaatgtgtctcctgagacaagcatcgtgatagagttcacagtcttaaaagtctgcattttcaggtggggcaaggtggctcatgcctttaatcccagcattttgggaggctaaggcagggagatggcttgagaccaggagttcaagaccagcctgggcaacatagtgagacgcccccccccccatctctacaaaaaatttaaaaaattagccatggtggtgtgcacctgtggtcccagctactccagaggctgaggtgggaagatcatttgagcccaggaggctgaggctgcaatgactgataattattgcaccactgaactccagcctgggccacatagcaagaccctgtctccaaaaaaaaaaaaaaaaaaaaaaaggaataaaggacgcagaatagggagaaaaacttctctcatatcatttatttggtccttcagtatttctgaatacttagtctaatttggcacaaaatcaactgtattagtccattcttgcaaagaaatacctgagactgggtattttataaggaaaagaggttggattggctcacgttctgcagctgcacagaagcatggcagcatctgcttctgggagacctcggggagtttttgctcatggtggaaggcgaagggggagcaggcgtcttgcggcaggagaaggaccaagagaggagaggaagagccgcacacttttaaacaaccagatctcgtgagaagctactccctccgcagcaccaagcgggggatggtgctcaaccattcatgagaactctgtccccatcattcagtcacctccccccaggccccaccgccgattctgaggatgacaattccacatgagatttgggcggggacacacatccaaactatattgtcaaccttctacacagccaatgacttaacagcctcattagaagttacagaaactaaagcaataaagttcggacaatgcagagaggtcaccttccttctatggccttgaagaaaccctggag

SEQ ID NO: 91 - DNA, 881 bp, Homo sapiens
cgctgtttgtctttctcggaaacaacagtaacgataagcctcttggaatatggaggccgctgcggacggcccggctgagacccaaagcccggtggaaaaagacagcccggcgaagacccaaagcccagcccaagacacctcaatcatgtcgagaaataacgcagatacaggcagagttcttgccttaccagagcacaagaagaagcgcaagggaaacttgccagccgagtccgttaagatcctccgcgactggatgtataagcatcggtttaaggcctacccttcagaagaagagaagcaaatgctgtcagagaagaccaatttgtctttgttgcagatttctaactggtttatcaatgctcgcagacgcattctcccggatatgcttcaacagcgtagaaacgaccccatcattggccacaaaacgggcaaagatgcccatgccacccacctgcagagcaccgaggcgtctgtgccggccaagtcagggcccagtggtccagacaatgtacaaagcctgcccctgtggcccttgccaaagggccagatgtcaagagagaagcaaccagatccggagtcggcccctagccagaagctcaccggaatagcccagccgaagaaaaaggtcaaggtttctgtcacatccccgtcttctccagaacttgtgtctccagaggagcacgccgacttcagcagcttcctgctgctagtcgatgcagcagtacaaagggctgccgagctggagctagagaagaagcaagagcctaatccatgattgatgatgttccaaaaacccaagtagtcagtcccttatgtactgtggtaaacctgtttatgttcaccccaaaaaaaaaaaaaaaaaaaaaaaaaaaaaaaaaaaa

SEQ ID NO: 92 - DNA, 2734 bp, Homo sapiens
cccgctccccgcaggggtggagcctgagcgaggagcccgcgagctctcctctctcctctggtctccgcggatgacgagtggctggataacatggagtcgggcaagatggcgcctcccaagaacgctccgagagatgccttggtgatggcacagatcctgaaggatatgggaatcacagagtatgaaccaagggttataaatcaaatgttggaatttgctttccgttatgtgactacaattctggatgatgcaaaaatttattcgagccatgctaagaaacctaatgttgatgcagatgatgtgagactggcaatccagtgtcgtgctgaccaatcttttacctctcctcccccaagagattttttactggatatcgcaaggcagaaaaatcaaacccctttgccactgattaagccatatgcaggacctagactgccacctgatagatactgcttaacagctccaaactataggctgaagtccttaattaaaaagggacctaaccaagggagactagttccacgattaagtgttggtgctgttagtagcaaacctactactcctactatagcaaccccacaaacggtgtctgtcccaaataaagttgcaactccaatgtcagtgacaagccaaagatttacggtgcagattccaccttctcagtccacacctgtcaaaccagttcctgcaacaactgcagttcaaaatgttctgattaatccttcaatgattgggcccaaaaatattcttattaccaccaacatggtttcgtcacagaacacagccaatgaagcaaacccactgaagagaaaacatgaagatgatgatgacaatgatattatgtaaggaatatagtctagtctagatgcatttcaaaaggaaagttggttttgagcccagtatactgaactagaatattctagatcttgttttaccttaaaagactgaaatgtagctgcagtatttttcgtcagttaaaactgttagatctctttagtaaacatacaaatgtgtttttcagtaggcttcaattacaaacagttaagtacttgtttcttaatagtttcattaatgttgagtcctaatatggcaattctgtttttgagacacttttctgccttctatcactgatagtagcatttaagtttcagattctacagcctagtcagcagatttatctttgacagcataatttctgatctacatttaaatactactttattttataaggtaatatagttacttactgtcactagctaattttttttttttaccaaatctctcaatataggaaacattttttggaaaggatggatttagaggagagattctaaatccattttggatgtatttgtttctttgacttccttaacttccaaactcaactccctttctagatcccattcattttctgcacctcccccataggcttgtttctctccattgctattaaatgtagaaggccatacctggaattttaaaaatattattcctggtaatacagctcagtgtcattttcctatttttaaaacatgctctacatgcctaatgtttcgtgattcactttaacctgatggtttgcatttgctgtttttcactcttatgtcagagcagttgggttttaccccttagtttttatgcctgttaagctttactgtgcttttgacaggtagttttgggtcagttatattcagttttagtattgtattccaagttgataactcttccatgtttcacatttctaaatttaacagagatgctgtagcttaaaacatgttttgataagtaattacactggacctaggcaaaaccactgaagaacaagtgttcctttttaccacatacatattatgttttgatcactgctgcttgagccccgctattggtataattcagatcatattagcttgttgctgaggatgtcacatataagacaggatcaaacctgaaagacactaatggttcatcctctataaggccaacaaagaaaacaaattaatgtgatgtaacttgcatttatgtgcactctgcataggtttcccaataatactgtaatgttgctgaacacctttccctgttttccatttatacacatattaaagtcatattgggaaagatggcaaagcttttgagaatataaacacacctgttttgtttttcttaagttcaacatacttttgtagaatttgtaaaataaatgttcgtatcataattgagaatttatagccaaggatcctcaaaacagaagtttcatcttaggatagtgtttgatatatctaagaaagtccagtaacagagaagaaatgtctcaaatgtctcgtatcagttttagtgtttttcagtattagggaaaggtaatataaaaacaggtaaaatgtataaaaatagacctgatcaacatcataggagataaatattcattatatagcataggatattattacttgaatgttctcagggaggcagcaagtcagaaggcctaggttctagtcctagctccatcatttaccaactgtgtgaccttacttcacctcttagccttgttctcctcactagcagtaggaaagtaacacctaccctgcctacctcacagggctgttgtgagggttcaattggtatatgtgaaaattctttcagaattgtaaaatgctatatgactgtaaggaattattgcatttgttccaaggttaataaaaatttgagcttaaaaaaaaaaaaaaaaaaaaaaaaaaaa

SEQ ID NO: 93 - DNA, 1721 bp, Homo sapiens
ggctcccacaccactgcctcgtgtggggttgttcgcccgtgaaggggcaggacagggtgcgcgctggtggaggttgaaattgttacattttggccgggcgcggtggctcacgcctgtaatcccagcactttgggaggccgaggcaggtggatcgcgaggtcaggagatcgagaccatcctggctggcacggtgaaaccccgtctctactaaaaaaatgcaaggaatcggccgggcgtggtgacgggcgcctgtggtcccagctgctcgggaggctgaggcaggaggatggcgtgaacccgggaggcggagcttgcagtgagccgagatcgcgccagtgcactccagcctgggcgacagagcgaaactacgtctcaaaaaaaaaaaaaaagaaagaaaagaaattgttacattttatatatataggaacaatcctgcaccatgactcaacagccacttcgaggagtgaccagcctgcgtttcaaccaagaccaaagctgcttttgctgcgccatggagacaggtgtgcgcatctacaacgtggagcccttgatggagaaggggcatctggaccacgagcaggtgggcagcatgggcttggtggagatgctgcaccgctccaaccttctggccttggtgggcggtggtagtagtcccaagttctcagagatctcagcagtgctgatctgggacgatgcccgggagggcaaggactccaaggagaagctggtgctggagttcaccttcaccaagccagtgctttctgtgcgcatgcgccatgacaagatcgtgatcgtgctgaagaaccgcatctatgtgtactccttccccgacaatccccgaaagctgtttgagtttgatacccgggacaaccccaaggggctctgtgacctctgccccagcctggagaagcaactgctagtgttcccgggacacaagtgtgggagtctgcaacttgtggacctggcgagcacaaagcctggcacctcgtctgctccattcacgatcaatgcacatcagagtgacatagcctgtgtgtctctaaaccagccaggcactgtagtggcctcagcctcccagaagggtacccttattcgcctctttgacacacaatccaaggagaaactggtggagctgcgccgaggcactgaccctgccaccctctactgcattaacttcagccacgactcctccttcctctgcgcttccagtgataagggtactgtccatatctttgctctcaaggatacccgcctcaaccgccgctccgcgctggctcgcgtgggcaaggtggggcctatgattgggcagtacgtggactctcagtggagcctggcgagcttcactgtgcctgctgagtcagcttgcatctgcgccttcggtcgcaatacttccaagaacgtcaactctgtcattgccatctgcgtagatgggaccttccacaaatatgtcttcactcctgatggaaactgcaacagagaggctttcgacgtgtaccttgacatctgtgatgatgatgacttttaaggaccctgggggctgtgctagggacctgcagtggcagaactgcagagctgagccttggcagtggggcgtgcttggaagccaccagccagcaagcattaatggggctggtgcccactttccactcagcagagctatgtctaaataaagagctcacttccccccaaaaaaaaaaaaaaaaaaaaaaaaaaaaaaaaaa

SEQ ID NO: 94 - DNA, 3921 bp, Homo sapiens
actcactatagggctcgagcggccgcccgggcaggtggggctccgcgggcctggagcacggccgggtctaatatgcccggagccgaggcgcgatgaaggagaagtccaagaatgcggccaagaccaggagggagaaggaaaatggcgagttttacgagcttgccaagctgctcccgctgccgtcggccatcacttcgcagctggacaaagcgtccatcatccgcctcaccacgagctacctgaagatgcgcgccgtcttccccgaaggtttaggagacgcgtggggacagccgagccgcgccgggcccctggacggcgtcgccaaggagctgggatcgcacttgctgcagactttggatggatttgtttttgtggtagcatctgatggcaaaatcatgtatatatccgagaccgcttctgtccatttaggcttatcccaggtggagctcacgggcaacagtatttatgaatacatccatccttctgaccacgatgagatgaccgctgtcctcacggcccaccagccgctgcaccaccacctgctccaagagtatgagatagagaggtcgttctttcttcgaatgaaatgtgtcttggcgaaaaggaacgcgggcctgacctgcagcggatacaaggtcatccactgcagtggctacttgaagatcaggcagtatatgctggacatgtccctgtacgactcctgctaccagattgtggggctggtggccgtgggccagtcgctgccacccagtgccatcaccgagatcaagctgtacagtaacatgttcatgttcagggccagccttgacctgaagctgatattcctggattccagggtgaccgaggtgacgggttacgagccgcaggacctgatcgagaagaccctataccatcacgtgcacggctgcgacgtgttccacctccgctacgcacaccacctcctgttggtgaagggccaggtcaccaccaagtactaccggctgctgtccaagcggggcggctgggtgtgggtgcagagctacgccaccgtggtgcacaacagccgctcgtcccggccccactgcatcgtgagtgtcaattatgtactcacggagattgaatacaaggaacttcagctgtccctggagcaggtgtccactgccaagtcccaggactcctggaggaccgccttgtctacctcacaagaaactaggaaattagtgaaacccaaaaataccaagatgaagacaaagctgagaacaaacccttaccccccacagcaatacagctcgttccaaatggacaaactggaatgcggccagctcggaaactggagagccagtccccctgcaagcgctgctgctcctccagaactgcagccccactcagaaagcagtgaccttctgtacacgccatcctacagcctgcccttctcctaccattacggacacttccctctggactctcacgtcttcagcagcaaaaagccaatgttgccggccaagttcgggcagccccaaggatccccttgtgaggtggcacgctttttcctgagcacactgccagccagcggtgaatgccagtggcattatgccaaccccctagtgcctagcagctcgtctccagctaaaaatcctccagagccaccggcgaacactgctaggcacagcctggtgccaagctacgaagcgcccgccgccgccgtgcgcaggttcggcgaggacaccgcgcccccgagcttcccgagctgcggccactaccgcgaggagcccgcgctgggcccggccaaagccgcccgccaggccgcccgggacggggcgcggctggcgctggcccgcgcggcacccgagtgctgcgcgcccccgacccccgaggccccgggcgcgccggcgcagctgcccttcgtgctgctcaactaccaccgcgtgctggcccggcgcggaccgctggggggcgccgcacccgccgcctccggcctggcctgcgctcccggcggccccgaggcggcgaccggcgcgctgcggctccggcacccgagccccgccgccacctccccgcccggcgcgcccctgccgcactacctgggcgcctcggtcatcatcaccaacgggaggtgacccgctggccgcccgcgccaggagcctggacccggcctcccggggctgcggcgccaccgagcccggcaaatgcgcacgacctacattaatttatgcagagacagctgtttgaattggaccccgccgccgacttgcggatttccaccgcggaggccccgcgcgccggtgccgagggccgaggagcgcccgggtccgggcaggtgaccgcccgcctctgtcctgcgagggccggtgcgacccagttgctgggggcttggtttcctcaccttgaaatcgggcttcacgcgtcttgccttgtccccaacgttccacaacagtcccgctgggggattgaagcggtttcactccgcaaatatcctccactttcaggagggaaaacccaccctaccacagtccgctcttccaagtggacggcagacctgggaggggacgcctgtgtcacgagcccttttagatgcttaggtgaaggcagaagtgatgattgtaagtcccatgaatacacaactccactgtctttaaaagtcattcaagagtctcattatttttgtttttatttaaccctttcttcaatacaaaaagccaacaaaccaagactaagggggtgaccatgcaattccattttgtgtctgtgaacataggtgtgcttcccaaatacattaacaagctcttacttccccctaacccctatgaactcttgataacaccaagagtagcaccttcagaatatattgaataggcattaaatgcaaaaatatatatgtagccagacagtttatgagaatgaccctgtcaagcttcattattacgtggcaaaatccctctggcccacacagatctgtaattcactaggctcgtgtttgctacaaatagtgctaataaagttaaattgcacgtgcaatacggaacactgtcaatggactgcaccttgtgaaggaaaaacatgcttaagggggtgtaatgaaaatgatgtagacattttaagcattttctacacagcgagaaaacttcgtaagaacatgttacgtgtgcaacaggtaaacagaaatcctttcataaagcaccagcagtgtttaaaaaatgagcttccattaatttttactttttatgggttttgcttaaagatctcaacatggaaaaatcctgtcatggctctgaactgcacaatgcattgaaccgccgtccttcaattttcttcacactatcaacactgcagcattttgctgctttatcaaaatggtttattttaggaaactttttccacctttctgaatggaaagaggttttcacaaatgttttaaactcatcgttctaaaatcaagtgcacctacaccaactgctctcaaaatgtgaactgacttttttttttttttttttgccaaccctgtgtcacttagtgaggacctgacacaatccctacagggtgtctgtcagtgggcctcatggtaagagtcacaatttgcaaatttaggaccgtgggtcatgcagcgaaggggctggatggtaggaagggatgtgcccgcctctccacgcactcagctatacctcattcacagctccttgtgagtgtgtgcacaggaaataagccgagggtattatttttttatgttcatgagtcttgtaattaaaccgtgattcttgaaaggtgtaggtttgattactaggagataccaccgacatttttcaataaagtactgcaaaatgcttttgtgtctaccttgttattaacttttggggctgtatttagtaaaaataaatcaaggctatcggagcagttcaataacaaaggttactgttgagaaaaaagaccctatcatagatttacaag

SEQ ID NO: 95 - DNA, 4007 bp, Homo sapiens
  misc_feature (125)..(125): n is a, c, g, or t
ggatccgcgcgaattttcaaagaacatattttccgttcacccccgctggtcttttactgccatcaatacactgttcttggtgcaaatacctcagcctctttattcaaagtatgttttatgttttngccaaatatgatctctaattgaaagtttatttttggttttggatgaatctgcggagcttaagttgtgagaagaaagggggaacaagacacaatgaaagaaaagtccaaaaatgctgcgcggactaggagggagaaggaaaacagcgaattttatgaactggctaaattactgcctttggcctcggctatcacctcgcaggtggacaaagcatccataatcagactcacgaccagctatctcaaaatgagagtggtgttcccagaagggctcggcgaggcgtggggccactcaagtcggaccagccccctggacaacgttggccgagaactgggctcccatctgctccagaccctggatggcttcatcttcgtggtagccccagatgggaagatcatgtacatctcagagacagcctcagtccacttgggtctttctcaggtagagctgaccggaaacagcatttatgaatacattcacccggcagaccacgacgagatgacggcggtgctcaccgcccatcaaccctaccactctcacttcgtgcaggagtatgagatcgagcgctccttcttcctgaggatgaagtgcgtcttggccaagcgtaacgccggcctcacctgtggcggctacaaggtcatccactgcagcggctacttgaagatccgccagtacagcctggacatgtcccccttcgacggctgctaccaaaacgtgggcctggtggccgtgggccactcgctgcctcccagcgccgtcacggagatcaagctacacagcaatatgtttatgttccgcgccagcctggacatgaagctcatctttctggactccagggtggcggagctgacggggtacgaacctcaggacctgattgagaagactctgtaccaccatgtgcacggctgcgacaccttccacctgcgctgcgcgcaccatttgctgctggtgaagggacaggtgaccaccaagtactacaggttcctggcgaaacacggcggctgggtatgggtgcaaagctacgcgaccatcgtgcacaacagtcgctcctccaggccacactgtatcgtcagcgtcaactatgtcctcacagacacagaatacaaagggctgcagctctccctggatcagatctcagcctccaaaccagccttctcctataccagcagctccacccccaccatgactgacaacagaaagggggccaaatcccggctctccagctcaaagtcaaaatccaggacttccccataccctcagtattcgggatttcacacagaaagatcggaatctgatcatgacagccagtggggcggaagtcccttgaccgacacggcctctccgcagcttctggaccccgccgataggcctggctcccagcacgacgcatcgtgcgcctacagacagttttcggaccgcagctctctctgctatggctttgcgcttgaccactcgaggctggtggaagagaggcatttccatacccaggcctgtgaaggaggccgatgtgaggcaggcaggtacttcctgggaacgccgcaggccgggagggagccctggtggggctctcgcgcagccttgcccctgacaaaggcctccccagaaagcagagaagcctatgaaaacagcatgcctcacatcgcttcagtccacaggatccatgggcgaggtcattgggatgaagatagtgtggtcagttctccagaccctgggtcggccagtgaatcaggtgaccgatatcgtactgagcagtatcaaagtagcccacatgaacccagcaaaattgaaactcttataagagccactcagcaaatgattaaagaagaagagaacagattacagctaaggaaagccccctcagaccaactggcttccattaatggggctgggaaaaaacactccctgtgttttgcaaactaccaacagcccccaccaacaggtgaagtctgccatggctctgctcttgccaacacttcaccatgtgaccatatccagcagagagagggaaaaatgttgagcccccatgaaaatgactatgacaacagtcccaccgcactatctcggataagtagtcccaattcggatcgcatttcaaaatccagtttgatcctagctaaagactatctgcattcggatatatctcctcatcagacagcaggagaccaccctactgtctctccaaactgctttggctctcaccggcagtattttgacaagcatgcttacacattaactggatatgccctggagcacttatatgacagcgaaaccattagaaactattccttgggctgtaatggctcacactttgatgtaacttcccatctgaggatgcaaccagacccagcacaaggacacaagggaacatctgttataataaccaacggaagctgatgttttgctgaaatattttgttctttaaggatctctgaaacatatttatagtttaataccccattaccagcatttactatgccacagattgttagagagtataacttaagttactgggtatttgatacgtgttcctataaaatcaaagaaaacatagcactagcattcagggttatacacagaaaagggagctaaattgaatacacaaatttcccctctaattatatgggaaccagaatagataaattttgacttgaaaaatattcatgtagatcaagtgtgcatatatactacatgagaggactgatgaatgacaacattgcattgtgactatccagtgatcctcaaacacacaaactattacttacaaactgcggtatacattttacatatggaaatataggctatgtaatgtaaatacatcaaaaatgggtaattttctttgactctgtcacactaaacttcttaacgaaatttccattcccaaaataactgagaaagagagagatacatcttataaactgacttctttgtggtttcaaatcagccagctcatttggttcaggcataaattagagaaatggttctggatatggtgcaaaaatgagttttcacctggtatccattataaacaatcaggaagaggtaatttttcaccttgcttttcagttagacaaggaccaggattgcactgacatggcgctgagggtttttctaagtaagaacactgagatattgggacacacatcaaaaacctggagtgctcaattggaagtagttctatgaatatggaaaggccagaggcagagtgaaataaaatgctatctcaaagtttaacacaatttaagggctcagcataagtaaacaacatatttggggtttgcttgtaaaaccaactaaataaaaaattcaaaccaattcacccagaaaaaagaccaataggtgcaaaaataaaaggaaaaccagtgaagtgccacatgacagcagtgttaagtgtttgaaaacgtttcaaagcacatatgtgccaatgtgacaacatgtggaaagcctcaggagagagtctaagataaaagcttaggctgatagacaagtagttaagagctaagagcagtactctgaaggaataggcaaaatgtttattttccttattgtttgtaaacaacaaacttggtcttacatctgtgtggtatagtagaaaggccagctgactagatctctggattctaattttggccctacctgtaacttaattttgtgaccacagttgtaccattcaccgtgcctgggctctagtttcctggtttgtaaggcagccccagcgttcatgttctgtgatagagcagaactgaacttattacctaattaactctctgctatgagttgtcaagactgatcattctgttttttctgtacacagaagtttagatgctttgtgacttaagcaggtgtgtgggctcctttaggcaggttacagttaatttctagagtcg

SEQ ID NO: 96 - DNA, 1821 bp, Homo sapiens
cgcgtgaactgcttcctgcaggctggccatggcgcttcacgttcccaaggctccgggctttgcccagatgctcaaggagggagcgaaacacttttcaggattagaagaggctgtgtatagaaacatacaagcttgcaaggagcttgcccaaaccactcgtacagcatatggaccaaatggaatgaacaaaatggttatcaaccacttggagaagttgtttgtgacaaacgatgcagcaactattttaagagaactagaagtacagcatcctgctgcaaaaatgattgtaatggcttctcatatgcaagagcaagaagttggagatggcacaaactttgttctggtatttgctggagctctcctggaattagctgaagaacttctgaggattggcctgtcagtttcagaggtcatagaaggttatgaaatagcctgcagaaaagctcatgagattcttcctaatttggtatgttgttctgcaaaaaaccttcgagatattgatgaagtctcatctctacttcgtacctccataatgagtaaacaatatggtaatgaagtatttctggccaagcttattgctcaggcatgcgtatctatttttcctgattccggccatttcaatgttgataacatcagagtttgtaaaattctgggctctggtatcagttcctcttcagtattgcatggcatggtttttaagaaggaaaccgaaggtgatgtaacatctgtcaaagatgcaaaaatagcagtgtactcttgtccttttgatggcatgataacagaaactaagggaacagtgttgataaagactgctgaagaattgatgaattttagtaagggagaagaaaacctcatggatgcacaagtcaaagctattgctgatactggtgcaaatgtcgtagtaacaggtggcaaagtggcagacatggctcttcattatgcaaataaatataatatcatgttagtgaggctaaactcaaaatgggatctccgaagactttgtaaaactgttggtgctacagctcttcctagattgacacctcctgtccttgaagaaatgggacactgtgacagtgtttacctctcagaagttggagatactcaggtggtggtttttaagcatgaaaaggaagatggcgccatttctaccatagtacttcgaggctctacagacaatctgatggatgacatagaaagggcagtagacgatggtgttaatactttcaaagttcttacaagggataaacgtcttgtacccggaggtggagcaacagaaattgaattagccaaacagatcacatcatatggagagacatgtcctggacttgaacagtatgctattaagaagtttgctgaggcatttgaagctattccccgcgcactggcagaaaactctggagttaaggccaatgaagtaatctctaaactttatgcagtacatcaagaaggaaataaaaacgttggattagatattgaggctgaagtccctgctgtaaaggacatgctggaagctggtattctagatacttacctgggaaaatattgggctatcaaactcgctactaatgctgcagtcactgtacttagagtggatcagatcatcatggcaaaaccagctggtgggcccaagcctccaagtgggaagaaagactgggatgatgaccaaaatgattgaaattggcttaatttttactgtaggtgaaggctgtatttgtagtagtactcaagaatcacctgatgttttcttattctccttaaattaagagttattttgtgtttgtattcttggctggatgttataataaacatattgttactgtc

SEQ ID NO: 97 - DNA, 4406 bp, Homo sapiens
ggaggctggaggggggttggggcttctcagcgccgattccgcgggaagggccctggggcctcacacttagtcccgggagctgcaggtcttacctggagagacgctgcacgtggatcccgcgccgctgcggttctcagccggctctggagtgcgggcggaggcgacagggccgattctggagtgggactgagcctttgaaatactccagccacgactaaaagagaagcagaggagctgatagaaattgagattgatggaacagagaaagcagagtgcacagaagaaagcattgtagaacaaacctacgcgccagctgaatgtgtaagccaggccatagacatcaatgaaccaataggcaatttaaagaaactgctagaaccaagactacagtgttctttggatgctcatgaaatttgtctgcaagatatccacctggatccagaacgaagtttatttgaccaaggagtaaaaacagatggaactgtacagcttagtgtacaggtaatttcttatcaaggaattgaaccaaagttaaacatccttgaaattgttaaacctgcggacactgttgaggttgttattgatccagatgcccaccatgctgaatcagaagcacatcttgttgaagaagctcaagtgataactcttgatggcacaaaacacatcacaaccatttcagatgaaacttcagaacaagtgacaagatgggctgctgcactggaaggctataggaaagaacaagaacgccttgggataccctatgatcccatacagtggtccacagaccaagtcctgcattgggtggtttgggtaatgaaggaattcagcatgaccgatatagacctcaccacactcaacatttcggggagagaattatgtagtctcaaccaagaagatttttttcagcgggttcctcagggagaaattctctggagtcatctggaacttctccgaaaatatgtattggcaagtcaagaacaacagatgaatgaaatagttacaattgatcaacctgtgcaaattattccagcatcagtgcaatctgctacacctactaccattaaagttataaatagtagtgtgaaggcagccaaagtacaaagagcgccgaggatttcagaagatagaagctcacctgggaacagaacaggaaacaatggccaaatccaactatgacagtttttgctagaacttcttactgataaggacgctcgagactgtatttcttgggttggtgataaaggtgaatttaagctaaatcagcctgaactggttgcacaaaaatggggacagcgtaaaaataagcctacgatgaactatgagaaactcagtcgtgcattaagatattattatgatggggacatgatttgtaaagttcaaggcaagagatttgtgtacaagtttgtctgtgacttgaagactcttactggatacagtgcagcggagttgaaccgtttggtcacagaatgtgaacagaagaaacttgcaaagatgcagctccatggaattgcccagccagtcacagcagtagctctggctactgcttctctgcaaacggaaaaggataattgagccccaggacattcggggactccaaagtctttcttaaaatgtttagagcaagtatagctcttacctttattactgaatttgaatcttcttttatttctaggctgtacagtctgatgcatgatttttttataaatatttcatactcttgtgaatttggatctttttattttgagcatatattttagaatatgtgtgtgttaaaggatctccacaatgtctgcggtgtgaaggcaggttcattgtggaatagtttgtcaacagtcaggaaagctaaactggtcagtattaatgtgtagccctaccaaaaatagccagtagtatctgaaaataaaaaataaatgaagtatctctaggaaacagtctggcttaactatatttgaaaatataactgtttcccctctctgctgctttagatgttgctttacatagaaccagaaaatggaatttctcagataaagcatgtgtgcctgtttcatctaatcaagcagagctaaaatgttcataccaaataaatttataataataaattactaaactaagagtatcaggttatttatatatttgcaagcaaaggacagtaagaagttggctggcaaaagagcagtgctgaaggaggagatccaggtttaaatctggcttattaactcaagccaattttaaggattttctgtatagattattcatgtcagaccaagaatttaaattattttgagagaggcatttaattctaataaaccagctgttacaaaaattataaaatgatctctgtttttcctgtcagagatttaaaaaactgaaaaggtatacctcaacccaaaaataaaggtttggtttggtttgttatggcttcctttttaaaaaaattaccctgtagtgccagtttattatgcaaagcagcttatattcctttgtttctgataaaatgaagactttaaatcagtcagcagtactttacctttcaaggcattagtaaattacttgcaaatagttttaaaaggaaaattcgacctctgttataggcagtcttctctttaagacaatacttttccacttattttttttccttttccatattatatatgtgtattcatatatctatatacatattcagttgatcattttataaacatatatgaaggcatgaagatatacagaagaaaaattattaaacaactcattttaagattcaaattaagtaattcctgcatatatgacattccttacataagcgaacactaaacaaaaatggctagaaatgtctttttctttcttttctctttttgttgtttgttttaaggtattaagcacgaattattacatgagactggcagatagctattaatcctcttacagatttgagaaagttgattctcaaatatttatgcaccttttccttcattgttttctttaaatatgtccccttaaaaagcttcttaagagctcagttaatgcttttgacttaactaggagaaaaagacatgataatacaggcaagatggcattgttagcaattctggtagtggtttggaatgaatcctaagaggcaggtatcttaaggacaaggaagagaagagagagaggaggaatctttgatctctttctctggtaatcttaacgcataattttactacaacatgttctcaattcatttatattattatattaagctctttctgcagttgatatctgggcagagtaagatttgtatttccatttttacttttttgaaagagaatatatggacagattattagtacaatttgggcactgtggttgtaagaatatctgagtaaaataacaatatgaaataataaacagaagctctagcgtcaggtaacaaatagacagcaagaaaggttttgcaccatcctcttacggcctagggagttgacaagttgcttgtagttttaaaaaaataataaagtatacccttctggtgtatcatcaagagcttaagaatcttggctttcatatttaaaatgcttttggggagacatatattaaaattttagccaagatgatagacatgtctcaattatatatgtgtgtgtatgtttttaaagctagaaacattacttttagattcctagaatgaaaacttttttctcatctatgcaattcccatatggtttttttaaaatcatattttattcattttctccctttagcaattttcattttatttctcataatttgaacagagacagttctcctacatgatcagatgctttttttttcttcttgccatcatttatgcatgacataggtaaagtaatatgactaatttctccagttgattcaagaaactcattactttgcctcaaattatatgtaaaatatttgttttacttaggttacagttatcagaaagccaggtagtttttttcttctattaaaatataacattgtgaaagaaaataaaatttattctattcattctttgctttgtttttataaatgaatttttcatagaatttacagtatattcaaaggaagaaagataaaattattggtcatcatttgtaccttagaagtacaagaatttaagtaaaagaaatgttcatttttgttttaaaatttgttttccatgtgaagtttttattgagccaactttcatacatatctcgctagcctaaagtctaaatatttgtgttggcatcagaaaaacaaattaggcagaattgctatgtgtggttgatcttcaggtaaattgactgatcacatttatttttgtatcagtctatgtcatttaattaggaaaaactgtttagttgttttctcccctgattaatggtgatactcaagtatgatacaaaaagaactgtaccaccaaa

SEQ ID NO: 98 - DNA, 1834 bp, Homo sapiens
gacagtaacaatatgtttattataacatccagccaagaatacaaacacaaaatacctcttaatgtaaggagaataagaaaacatcacgcgattcttagaatactactacaaatacagccttcacctacagtaaaaattaagccaatttcaatcattttggtcatcatcccagtctttcttcccacttggaggcttgggcccaccatctggttttgccatgattacctgacccactctaagtacagtgactgcagcattagcagcgagtttgatagaccagtgttttcccaggtaagtatctagaacaccagcttccaacatgtccgttacagcagggactacagcctcagtatctaatccaacatttttatttccttcttgaggtactgcataaagtttagagattacttcattggccttaactccagagttttctccagagtatttctgccagtgcacggggaatagcttcaaacgcctcagcaaacttcttaatagcgtactgttcaagtccaggacatgtctctccatatgatgtgatctgtttggctaattcaatttctgttgctccacctccgggtacaagacgtttatcccttgtaagaactttgaaagtattaacaccatcatctactgccctttctatgtcatccatcagattgtctgtagagccctgaagtactatggtagaaatgatgccatcttccttttcatgcttaaaaaccaccacctgagtatctccaacttctgagaggtaaacactgtctcagtgtcccatttcttcaaggacaggaggtgtcaatctaggaagagctgtagcaccaactgttttacagagtcttcagacatcccattttgagtttagcttcactaacatcatattatatttgtttgcataatgaagagccatgtctgccactttgccacctgttactacaacatttgcaccagtatcagcaatagctttgacttatgcatccatgagattttcttctcccttacttaaattcatcaattcttcatcagtctttatcaacactgttcccttagtttctgttatcatgccatcaaaaggacaagagtacactgctatttttgcatctttgacagatgtacatcaccttctgtttccttcttaaaaaccatgccatgcaatactgaagaggaagtgataccacagcccagaattttacaaactctgatgttatcaactttgaaatggccagaatcaggaaaaatagatacgcatgcctgaacaataagcttggccagaaatacttcattaccatattgtttacacattacagaggtacgaagtagagatgagacttcatcaacatctcgaaggttttttgcagaacaacgtaccaaattaggaagaatctcatgagcttttctgcaagctatttcataaccttctatgacctctgaaactgacaggccaatcctcagaagttcttcagctaattccaggagagctccagcaaataccagaacaatgtttgtgccatctccaacttcttgctcttgcatatgagaagccattacagtcatttttgcagcaggatgctgtacttctagttctcttaaaatagtcgctgcatcatttgtcacaaacaacttctccaagtagttgataaccatttttttcattccatttcgtccatatgctgtacgagtggtttgggcaagctccttgcaagcttgtatgtttctatacacagcctcttctaattctgaaaagtgtttcgctccctccttgagcatctgggcgaagcccggagccttgggaacttgaagcgccatggccagcctgcaggaagccgttcacgtg

(19) United States
(12) Patent Application Publication
(10) Pub. No.: US 2005/0037388 A1
(43) Pub. Date: Feb. 17, 2005

(54) Method for detecting diseases caused by chromosomal imbalances

(75) Inventors: Stylianos Antonarakis, Geneva (CH); Samuel Deutsch, Geneva (CH)

Correspondence Address: PALMER & DODGE, LLP;PAULA CAMPBELL EVANS, 111 HUNTINGTON AVENUE, BOSTON, MA, 02199, US

(73) Assignee: University of Geneva

(21) Appl. No.: 10/852,943

(22) Filed: May 25, 2004

Related U.S. Application Data
(63) Continuation-in-part of application No. 10/177,063, filed on Jun. 21, 2002, now abandoned.
(60) Provisional application No. 60/300,266, filed on Jun. 22, 2001.

(51) Int. Cl. C12Q 1/68; C12P 19/34

(52) U.S. Cl. 435/6; 435/91.2

(57) ABSTRACT

The invention provides a universal method to detect the presence of chromosomal abnormalities by using paralogous genes as internal controls in an amplification reaction. The method is rapid, high throughput, and amenable to semi-automated or fully automated analyses. In one aspect, the method comprises providing a pair of primers which can specifically hybridize to each of a set of paralogous genes under conditions used in amplification reactions, such as PCR. Paralogous genes are preferably on different chromosomes but may also be on the same chromosome (e.g., to detect loss or gain of different chromosome arms). By comparing the amount of amplified products generated, the relative dose of each gene can be determined and correlated with the relative dose of each chromosomal region and/or each chromosome, on which the gene is located.

### RELATED APPLICATIONS

This application claims priority to provisional U.S. Application Ser. No. 60/300,266, filed on Jun. 22, 2001. This application is a Continuation-in-Part which claims priority under 35 U.S.C. § 120 to U.S. patent application Ser. No. 10/177,063 filed Jun. 21, 2002, the entirety of which is incorporated herein by reference.

### FIELD OF THE INVENTION

The invention relates to methods for detecting diseases caused by chromosomal imbalances.

### BACKGROUND OF THE INVENTION

Chromosome abnormalities in fetuses typically result from aberrant segregation events during meiosis caused by misalignment and non-disjunction of chromosomes. While sex chromosome imbalances do not impair viability and may not be diagnosed until puberty, autosomal imbalances can have devastating effects on the fetus. For example, autosomal monosomies and most trisomies are lethal early in gestation (see, e.g., Epstein, 1986, The Consequences of Chromosome Imbalance: Principles, Mechanisms and Models, Cambridge Univ. Press).

Some trisomies do survive to term, although with severe developmental defects. Trisomy 21, which is associated with Down Syndrome (Lejeune et al., 1959, C. R. Acad. Sci. 248: 1721-1722), is the most common cause of mental retardation in all ethnic groups, affecting 1 out of 700 live births. While parents of Down syndrome children generally do not have chromosomal abnormalities themselves, there is a pronounced maternal age effect, with risk increasing as maternal age progresses (Yang et al., 1998, Fetal Diagn. Ther. 13(6): 361-366).

Diagnosis of chromosomal imbalances such as trisomy 21 has been made possible through the development of karyotyping and fluorescent in situ hybridization (FISH) techniques using chromosome-specific probes. Although highly accurate, these methods are labor intensive and time consuming, particularly in the case of karyotyping which requires several days of cell culture after amniocentesis is performed to obtain sufficient numbers of fetal cells for analysis. Further, the process of examining metaphase chromosomes obtained from fetal cells requires the subjective judgment of highly skilled technicians.

Many methods have been proposed over the years to replace traditional karyotyping and FISH methods, although none has been widely used. These can be grouped into three main categories: detection of aneuploidies through the use of short tandem repeats (STRs); PCR-based quantitation of chromosomes using a synthetic competitor template, and hybridization-based methods.

STR-based methods rely on detecting changes in the number of STRs in a chromosomal region of interest to detect the presence of an extra or missing chromosome (see, e.g., WO 9403638). Chromosome losses or gains can be observed by detecting changes in ratios of heterozygous STR markers using polymerase chain reaction (PCR) to quantitate these markers. For example, a ratio of 2:1 of one STR marker with respect to another will indicate the likely presence of an extra chromosome, while a 0:1 ratio, or homozygosity, for a marker can provide an indication of chromosome loss. However, certain individuals also will be homozygous as a result of recombination events or non-disjunction at meiosis II and the test will not distinguish between these results. The quantitative nature of STR-based methods is also suspect because each STR marker has a different number of repeats and the amplification efficiency of each marker is therefore not the same. Further, because STR markers are highly polymorphic, the creation of a diagnostic assay universally applicable to all individuals is not possible.

Competitor nucleic acids also have been used in PCR-based assays to provide an internal control through which to monitor changes in chromosome dosage. In this type of assay, a synthetic PCR template (competitor) having sequence similarity with a target (i.e., a genomic region on a chromosome) is provided, and competitor and target nucleic acids are co-amplified using the same primers (see, e.g., WO 9914376; WO 9609407; WO 9409156; WO 9102187; and Yang et al., 1998, Fetal Diagn. Ther. 13(6): 361-6). Amplified competitor and target nucleic acids can be distinguished by introducing modifications into the competitor, such as engineered restriction sites or inserted sequences which introduce a detectable difference in the size and/or sequence of the competitor. By adding the same amount of competitor to a test sample and a control sample, the dosage of a target genomic segment can be determined by comparing the ratio of amplified target to amplified competitor nucleic acids. However, since competitor nucleic acids must be added to the samples being tested, there is inherent variability in the assay stemming from variations in sample handling. Such variations tend to be magnified by the exponential nature of the amplification process which can magnify small starting differences between a competitor and target template and diminish the reliability of the assay.

Some hybridization-based methods rely on using labeled chromosome-specific probes to detect differences in gene and/or chromosome dosage (see, e.g., Lapierre et al., 2000, Prenat. Diagn. 20(2): 123-131; Bell et al., 2001, Fertil. Steril. 75(2): 374-379; WO 0024925; and WO 9323566). Other hybridization-based methods, such as comparative genome hybridization (CGH), evaluate changes throughout the entire genome. For example, in CGH analysis, test samples comprising labeled genomic DNA containing an unknown dose of a target genomic region and control samples comprising labeled genomic DNA containing a known dose of the target genomic region are applied to an immobilized genomic template and hybridization signals produced by the test sample and control sample are compared. The ratio of signals observed in test and control samples provides a measure of the copy number of the target in the genome. Although CGH offers the possibility of high throughput analysis, the method is difficult to implement since normalization between the test and control sample is critical and the sensitivity of the method is not optimal.

A method which relies on hybridization to two different target sequences in the genome to detect trisomy 21 is described by Lee et al., 1997, Hum. Genet. 99(3): 364-367. The method uses a single pair of primers to simultaneously amplify two homologous phosphofructokinase genes, one on chromosome 21 (the liver-type phosphofructokinase gene, PFKL-CH21) and one on chromosome 1 (the human muscle-type phosphofructokinase gene, PFKM-CH1). Amplification products corresponding to each gene can be distinguished by size. However, although Lee et al. report that samples from trisomic and disomic (i.e., normal) individuals were distinguishable using this method, the ratio of PFKM-CH1 and PFKL-CH21 amplification observed was 1/3.3 rather than the expected 1/1.5, indicating that the two homologous genes were not being amplified with the same efficiency. Further, amplification values obtained from samples from normal and trisomic individuals partially overlapped at their extremes, making the usefulness of the test as a diagnostic tool questionable.

### SUMMARY OF THE INVENTION

The present invention provides a high throughput method for detecting chromosomal abnormalities. The method can be used in prenatal testing as well as to detect chromosomal abnormalities in somatic cells (e.g., in assays to detect the presence or progression of cancer). The method can be used to detect a number of different types of chromosome imbalances, such as trisomies, monosomies, and/or duplications or deletions of chromosome regions comprising one or more genes.

In one aspect, the invention provides a method for detecting risk of a chromosomal imbalance. The method comprises simultaneously amplifying a first sequence at a first chromosomal location to produce a first amplification product and amplifying a second sequence at a second chromosomal location to produce a second amplification product. The relative amount of amplification products is determined and a ratio of first to second amplification products when different from 1:1 is indicative of a risk of a chromosomal imbalance. Preferably, the first and second sequence are paralogous sequences located on different chromosomes, although in some aspects, they are located on the same chromosome (e.g., on different arms). The first and second amplification products comprise greater than about 80% identity, and preferably, are substantially identical in length. Because the amplification efficiency of the first and second sequences is substantially the same, the method is highly quantitative and reliable.

Amplification preferably is performed by PCR using a single pair of primers to amplify both the first and second sequences. In one aspect, the primers are coupled with a first member of a binding pair for binding to a solid support on which a second member of a binding pair is bound, the second member being capable of specifically binding to the first member. Providing the solid support enables primers and amplification products to be captured on the support to facilitate further procedures such as sequencing. In one aspect, primers are bound to the support prior to amplification. In another aspect, primers are bound to the support after amplification.

The first and second amplification products have at least one nucleotide difference between them located at an at least one nucleotide position thereby enabling the first and second amplification products to be distinguished on the basis of this sequence difference. Therefore, in one aspect, the method further comprises the steps of (i) identifying a first nucleotide at the at least one nucleotide position in the first amplification product, (iii) identifying a second nucleotide at the at least one nucleotide position in said second amplification product, and (iii) determining the relative amounts of the first and second nucleotides. The ratio of the first and second nucleotide is proportional to the dose of the first and second sequences in the sample. The steps of identifying and determining can be performed by sequencing. In a preferred embodiment, a pyrosequencing™ sequencing method is used.

In one aspect, the invention provides a method of detecting risk of trisomy 21 and the likelihood that the individual has Down syndrome by providing a first sequence on chromosome 6 and a second sequence on chromosome 21. In a preferred aspect, the first sequence comprises the SIM1 sequence, while the second sequence comprises the SIM2 sequence. Amplification is performed using a single pair of primers specifically hybridizing to identical sequences in both genes, such as primers SIMAF (GCAGTGGCTACTTGAAGAT) and SIMAR (TCTCGGTGATGGCACTGG). A ratio of amplified SIM1 and SIM 2 sequences of about 1:1.5 indicates an individual at risk for trisomy 21 or Down Syndrome.

In another aspect, the invention provides a method of detecting risk of trisomy 21 and the likelihood that the individual has Down syndrome by providing a first sequence on chromosome 7 and a second sequence on chromosome 21. In a preferred aspect, the first sequence comprises a GABPA gene paralogue sequence, while the second sequence comprises the GABPA sequence. In one aspect, the first sequence comprises the GABPA gene paralogue sequence presented in FIG. 3. Amplification is performed using a single pair of primers specifically hybridizing to identical sequences in both genes, such as primers GABPAF (CTTACTGATAAGGACGCTC) and GABPAR (CTCATAGTTCATCGTAGGCT). A ratio of amplified GABPA gene paralogue sequence and GABPA of about 1:1.5 indicates an individual at risk for trisomy 21 or down syndrome.

In another aspect, the invention provides a method of detecting risk of trisomy 21 and the likelihood that the individual has Down syndrome by providing a first sequence on chromosome 1 and a second sequence on chromosome 21. In a preferred aspect, the first sequence comprises a CCT8 gene paralogue sequence, while the second sequence comprises the CCT8 sequence. In one aspect the first sequence comprises the CCT8 gene paralogue sequence presented in FIG. 4. Amplification is performed using a single pair of primers specifically hybridizing to identical sequences in both genes, such as primers CCT8F (ATGAGATTCTTCCTAATTTG) and CCT8R (GGTAATGAAGTATTTCTGG). A ratio of amplified CCT8 gene paralogue and CCT8 of about 1:1.5 indicates an individual at risk for trisomy 21 or down syndrome.

In another aspect, the invention provides a method of detecting risk of trisomy 21 and the likelihood that the individual has Down syndrome by providing a first sequence on chromosome 2 and a second sequence on chromosome 21, wherein said second sequence comprises C21ORF19. In one aspect, the first sequence comprises a C21ORF19 gene paralogue sequence.

In another aspect, the invention provides a method of detecting risk of trisomy 21 and the likelihood that the individual has Down syndrome by providing a first sequence on chromosome 2 and a second sequence on chromosome 21, wherein said second sequence comprises DSCR3. In one aspect, the first sequence comprises a DSCR3 gene paralogue sequence.

In another aspect, the invention provides a method of detecting risk of trisomy 21 and the likelihood that the individual has Down syndrome by providing a first sequence on chromosome 4 and a second sequence on chromosome 21, wherein said second sequence comprises C21Orf6. In one aspect, the first sequence comprises a C21Orf6 gene paralogue sequence.

In another aspect, the invention provides a method of detecting risk of trisomy 21 and the likelihood that the individual has Down syndrome by providing a first sequence on chromosome 12 and a second sequence on chromosome 21, wherein said second sequence comprises WRB1. In one aspect, the first sequence comprises a WRB1 gene paralogue sequence.

In another aspect, the invention provides a method of detecting risk of trisomy 21 and the likelihood that the individual has Down syndrome by providing a first sequence on chromosome 7 and a second sequence on chromosome 21, wherein said second sequence comprises KIAA0958. In one aspect, the first sequence comprises a KIAA0958 gene paralogue sequence.

In another aspect, the invention provides a method of detecting risk of trisomy 21 and the likelihood that the individual has Down syndrome by providing a first sequence on the X chromosome and a second sequence on chromosome 21, wherein said second sequence comprises TTC3. In one aspect, the first sequence comprises a TTC3 gene paralogue sequence.

In another aspect, the invention provides a method of detecting risk of trisomy 21 and the likelihood that the individual has Down syndrome by providing a first sequence on chromosome 5 and a second sequence on chromosome 21, wherein said second sequence comprises ITSN1. In one aspect, the first sequence comprises an ITSN1 gene paralogue sequence.

In another aspect, the invention provides a method of detecting risk of trisomy 13 by providing a first sequence on chromosome 3 and a second sequence on chromosome 13. In a preferred aspect, the first sequence comprises a RAP2A gene paralogue sequence, while the second sequence comprises the RAP2A sequence. Amplification is performed using a single pair of primers specifically hybridizing to identical sequences in both genes. In one aspect, the RAP2A gene paralogue sequence comprises the RAP2A gene paralogue sequence presented in FIG. 5.

In another aspect, the invention provides a method of detecting risk of trisomy 13 by providing a first sequence on chromosome 2 and a second sequence on chromosome 13. In a preferred aspect, the first sequence comprises a CDK8 gene paralogue sequence, while the second sequence comprises the CDK8 sequence. Amplification is performed using a single pair of primers specifically hybridizing to identical sequences in both genes. In one aspect, the CDK8 gene paralogue sequence comprises the CDK8 gene paralogue sequence presented in FIG. 7.

In another aspect, the invention provides a method of detecting risk of trisomy 18 by providing a first sequence on chromosome 2 and a second sequence on chromosome 18. In a preferred aspect, the first sequence comprises an ACAA2 gene paralogue sequence, while the second sequence comprises the ACAA2 sequence. Amplification is performed using a single pair of primers specifically hybridizing to identical sequences in both genes. In one aspect, the ACAA2 gene paralogue sequence comprises the ACAA2 gene paralogue sequence presented in FIG. 8.

In another aspect, the invention provides a method of detecting risk of trisomy 18 by providing a first sequence on chromosome 9 and a second sequence on chromosome 18. In a preferred aspect, the first sequence comprises an ME2 gene paralogue sequence, while the second sequence comprises the ME2 sequence. Amplification is performed using a single pair of primers specifically hybridizing to identical sequences in both genes. In one aspect, the ME2 gene paralogue sequence comprises the ME2 gene paralogue sequence presented in FIG. 6.

In another aspect, the invention provides a method for detecting risk of a chromosomal imbalance, wherein the chromosomal imbalance is selected from the group consisting of Trisomy 21, Trisomy 13, Trisomy 18, Trisomy X, XXY and XO.

In another aspect, the invention provides a method for detecting risk of a chromosomal imbalance, wherein the chromosomal imbalance is associated with a disease selected from the group consisting of Down's Syndrome, Turner's Syndrome, Klinefelter Syndrome, William's Syndrome, Langer-Giedon Syndrome, Prader-Willi, Angelman's Syndrome, Rubenstein-Taybi and Di George's Syndrome.

In another aspect, the invention provides a method of detecting risk of trisomy 21 by providing a first sequence on chromosome 5 and a second sequence on chromosome 21. In a preferred aspect, the first sequence comprises the sequence of an intersectin (ITSN) paralogue and the second sequence comprises the sequence of intersectin (ITSN). In one aspect the intersectin paralogue comprises the sequence presented in FIG. 18. Amplification is performed using a single pair of primers specifically hybridizing to identical sequences in both genes, such as primers ITSNF (ATTATTGCCATGTACACTT) and ITSNR (GAATCTTTAAGCCTCACATAG).

In another aspect, the invention provides a method of detecting risk of trisomy 21 by providing a first sequence on chromosome 7 and a second sequence on chromosome 21. In a preferred aspect, the first sequence comprises the sequence of a GABPA paralogue and the second sequence comprises the sequence of GABPA. In one aspect the GABPA paralogue comprises the sequence presented in FIG. 19. Amplification is performed using a single pair of primers specifically hybridizing to identical sequences in both genes, such as primers GABPAF (CTTACTGATAAGGACGCTC) and GABPAR (CTCATAGTTCATCGTAGGCT).

In another aspect, the invention provides a method of detecting risk of trisomy 13 by providing a first sequence on chromosome 6 and a second sequence on chromosome 13. In a preferred aspect, the first sequence comprises the sequence of a NUFIP1 paralogue and the second sequence comprises the sequence of NUFIP1. In one aspect the NUFIP1 paralogue comprises the sequence presented in FIG. 20. Amplification is performed using a single pair of primers specifically hybridizing to identical sequences in both genes, such as primers NUFIP1F (GCTGAGCCGACTAGTGATT) and NUFIP1R (AAGGGAAGCGAGGACGTAA).

In another aspect, the invention provides a method of detecting risk of trisomy 13 by providing a first sequence on chromosome 6 and a second sequence on chromosome 13. In a preferred aspect, the first sequence comprises the sequence of an STK24F paralogue and the second sequence comprises the sequence of STK24. In one aspect the STK24R paralogue comprises the sequence presented in FIG. 21. Amplification is performed using a single pair of primers specifically hybridizing to identical sequences in both genes, such as primers STK24F(CGCTCTCGTCTGACATTT) and STK24R (TCAGACATTTTTAGGTGG).

In another aspect, the invention provides a method of detecting risk of trisomy 18 by providing a first sequence on chromosome 3 and a second sequence on chromosome 18. In a preferred aspect, the first sequence comprises the sequence of a KIAA1328 paralogue and the second sequence comprises the sequence of KIAA1328. In one aspect the KIAA1328 paralogue comprises the sequence presented in FIG. 22. Amplification is performed using a single pair of primers specifically hybridizing to identical sequences in both genes, such as primers KIAA1328F (CGAAGGAAATGTCAGATCAA) and KIAA1328R (GACTCCATGGAGATTGAAG).

In another aspect, the invention provides a method of detecting risk of trisomy 18 by providing a first sequence on chromosome 12 and a second sequence on chromosome 18. In a preferred aspect, the first sequence comprises the sequence of a WBP11 paralogue and the second sequence comprises the sequence of WBP11. In one aspect the WBP11 paralogue comprises the sequence presented in FIG. 23. Amplification is performed using a single pair of primers specifically hybridizing to identical sequences in both genes, such as primers WBP11F (GGAGGGACGGGAAGTAGAG) and WBP11R (GTGAAGAAGCAGTGGATGTGCC)

In another aspect, the invention provides a method of detecting risk of sex chromosome abnormalities by providing a first sequence on chromosome Y and a second sequence on chromosome X. In a preferred aspect, the first sequence comprises the sequence of An ARSD paralogue and the second sequence comprises the sequence of ARSD. In one aspect the ARSD paralogue comprises the sequence presented in FIG. 24. Amplification is performed using a single pair of primers specifically hybridizing to identical sequences in both genes, such as primers ARSDF (CGCCAGCAATGGATAC) and ARSDR (TGCAAAAGTGGTTTCGTTC).

In another aspect, the invention provides a method of detecting risk of sex chromosome abnormalities by providing a first sequence on chromosome Y and a second sequence on chromosome X. In a preferred aspect, the first sequence comprises the sequence of a TGIF2LX paralogue and the second sequence comprises the sequence of TGIF2LX. In one aspect the TGIF2LX paralogue comprises the sequence presented in FIG. 25. Amplification is performed using a single pair of primers specifically hybridizing to identical sequences in both genes, such as primers TGIF2LXF (AAGACAGCCCGGCGAAGA) and TGIF2LXR (ATTCCGGGAGAATGCGTCTGC).

In another aspect, the invention provides a method of detecting risk of sex chromosome abnormalities by providing a first sequence on chromosome 3 and a second sequence on chromosome X. In a preferred aspect, the first sequence comprises the sequence of a TAF9L paralogue and the second sequence comprises the sequence of TAF9L. In one aspect the TAF9L paralogue comprises the sequence presented in FIG. 26. Amplification is performed using a single pair of primers specifically hybridizing to identical sequences in both genes, such as primers TAF9LF (TGCCTAATGTTTTGTGATT) and TA9LR (GACCCAAAACTACCTGTC).

In another aspect, the invention provides a method of detecting risk of sex chromosome abnormalities by providing a first sequence on chromosome X and a second sequence on chromosome 4. In a preferred aspect, the first sequence comprises the sequence of a JM5 paralogue and the second sequence comprises the sequence of JM5. In one aspect the JM5 paralogue comprises the sequence presented in FIG. 27. Amplification is performed using a single pair of primers specifically hybridizing to identical sequences in both genes, such as primers JM5F (CCCTGTGTGTCTCTAAACCAGC) and JM5R (GGTGGCAGGGTCAGT).

### BRIEF DESCRIPTION OF THE DRAWINGS

The objects and features of the invention can be better understood with reference to the following detailed description and accompanying drawings.

FIG. 1 shows a partial sequence alignment of the SIM1 and SIM2 paralogs located on chromosome 6 and chromosome 21, respectively.

FIG. 2 shows allele ratios of SIM1 and SIM2 paralogs in Down syndrome individuals and normal individuals.

FIG. 3 shows the sequence alignment of the GABPA gene and a GABPA gene paralogue sequence. The first sequence corresponds to chromosome 21 and the second sequence corresponds to chromosome 7. The assayed nucleotide is shaded and indicated with an arrow.

FIG. 4 shows the sequence alignment of the CCT8 gene and a CCT8 gene paralogue sequence. The first sequence corresponds to chromosome 21 and the second sequence corresponds to chromosome 1. The assayed nucleotide is shaded and indicated with an arrow.

FIG. 5 shows the sequence alignment of the RAP2A gene and a RAP2A gene paralogue sequence. The first sequence corresponds to chromosome 13 and the second sequence corresponds to chromosome 3. The assayed nucleotide is shaded and indicated with an arrow.

FIG. 6 shows the sequence alignment of the ME2 gene and an ME2 gene paralogue sequence. The first sequence corresponds to chromosome 18 and the second sequence corresponds to chromosome 9. The assayed nucleotide is shaded and indicated with an arrow.

FIG. 7 shows the sequence alignment of the CDK8 gene and a CDK8 gene paralogue sequence. The first sequence corresponds to chromosome 13 and the second sequence corresponds to chromosome 2.

FIG. 8 shows the sequence alignment of the ACAA2 gene and an ACAA2 gene paralogue sequence. The first sequence corresponds to chromosome 18 and the second sequence corresponds to chromosome 2.

FIG. 9 illustrates the principle of the method of the invention.

FIG. 10 is an example of a blast result showing the ITSN1 gene on chromosome 21 and its paralogue on Chromosome 5 represented as a genome view.

FIG. 11 shows the result of a GABPA pilot experiment. Panel A shows an example of a pyrogram, with a clear discrimination between control and trisomic sample. See ratio between peaks at the position indicated by the arrow. G peak represents chromosome 21. Panel B shows a plot of G peak values (chromosome 21) for a series of 24 control and affected subject DNAs. Panel C is a summary of data.

FIG. 12 shows the primers used, as well as the position (circled) which was used for quantification in a GABPA optimized assay.

FIG. 13 shows the distribution of G values for the 230 samples analyzed in a GABPA assay. The G allele represents the relative proportion of chromosome 21.

FIG. 14 shows typical pyrogram programs for the GABPA assay. Arrows indicate positions used for chromosome quantification.

FIG. 15 shows the primers used, as well as the position (circled) which was used for quantification in a CCT8 optimized assay.

FIG. 16 shows the results of a CCT8 assay. The distribution of T values for the 190 samples analyzed are presented. The T allele represents the proportion of chromosome 21.

FIG. 17 shows typical pyrogram programs for the CCT8 assay. Arrows indicate positions used for chromosome quantification.

FIG. 18 shows the sequence alignment of the intersectin gene and an intersectin gene paralogue sequence. The first sequence corresponds to chromosome 21 and the second sequence corresponds to chromosome 5. Upper, lower and sequencing primers are indicated. The assayed nucleotide is indicated with an arrow. Additional potentially relevant nucleotides according to the method of the invention are circled.

FIG. 19 shows the sequence alignment of the GABPA gene and a GABPA gene paralogue sequence. The first sequence corresponds to chromosome 21 and the second sequence corresponds to chromosome 7. Upper, lower and sequencing primers are indicated. The assayed nucleotide is indicated with an arrow. Additional potentially relevant nucleotides according to the method of the invention are circled.

FIG. 20 shows the sequence alignment of the NUFIP1 gene and an NUFIP1 gene paralogue sequence. The first sequence corresponds to chromosome 13 and the second sequence corresponds to chromosome 6. Upper, lower and sequencing primers are indicated. The assayed nucleotide is indicated with an arrow. Additional potentially relevant nucleotides according to the method of the invention are circled.

FIG. 21 shows the sequence alignment of the STK4 gene and a STK24 gene paralogue sequence. The first sequence corresponds to chromosome 13 and the second sequence corresponds to chromosome 6. Upper, lower and sequencing primers are indicated. The assayed nucleotide is indicated with an arrow. Additional potentially relevant nucleotides according to the method of the invention are circled.

FIG. 22 shows the sequence alignment of the KIAA1328 gene and a KIAA1328 gene paralogue sequence. The first sequence corresponds to chromosome 18 and the second sequence corresponds to chromosome 3. Upper, lower and sequencing primers are indicated. The assayed nucleotide is indicated with an arrow. Additional potentially relevant nucleotides according to the method of the invention are circled.

FIG. 23 shows the sequence alignment of the WBP11 gene and a WBP11 gene paralogue sequence. The first sequence corresponds to chromosome 18 and the second sequence corresponds to chromosome 12. Upper, lower and sequencing primers are indicated. The assayed nucleotide is indicated with an arrow. Additional potentially relevant nucleotides according to the method of the invention are circled.

FIG. 24 shows the sequence alignment of the ARSD gene and an ARSD gene paralogue sequence. The first sequence corresponds to chromosome X and the second sequence corresponds to chromosome Y. Upper, lower and sequencing primers are indicated. The assayed nucleotide is indicated with an arrow. Additional potentially relevant nucleotides according to the method of the invention are circled.

FIG. 25 shows the sequence alignment of the TGIF2LX gene and a TGIF2LX gene paralogue sequence. The first sequence corresponds to chromosome X and the second sequence corresponds to chromosome Y. Upper, lower and sequencing primers are indicated. The assayed nucleotide is indicated with an arrow. Additional potentially relevant nucleotides according to the method of the invention are circled.

FIG. 26 shows the sequence alignment of the TAF9L gene and a TAF9L gene paralogue sequence. The first sequence corresponds to chromosome X and the second sequence corresponds to chromosome 3. Upper, lower and sequencing primers are indicated. The assayed nucleotide is indicated with an arrow. Additional potentially relevant nucleotides according to the method of the invention are circled.

FIG. 27 shows the sequence alignment of the JM5 gene and a JM5 gene paralogue sequence. The first sequence corresponds to chromosome X and the second sequence corresponds to chromosome 4. Upper, lower and sequencing primers are indicated. The assayed nucleotide is indicated with an arrow. Additional potentially relevant nucleotides according to the method of the invention are circled.

FIG. 28 shows the paralogous gene quantification principle. Panel A shows an ideogram of human chromosomes. The black horizontal bars highlighted by circles, show the position of paralogous sequences in the human genome. Only sequences that were present only twice, with a high degree of homology were used. Panel B shows a typical alignment between paralogous sequences used for designing paralogous sequence quantification (PSQ) assays. Dotted boxes indicate the position of primers, and the encircled position shows the Paralogous sequence mismatch used for quantification. Panel C shows the principle of the method. If a cell contains 2 copies of chromosome 5 and 2 copies of chromosome 21, one expects to see a ratio of 1:1 at the the paralogous sequences (PSM) position. When 3 copies of chromosome 21 are present this ratio should be 1.5:1.

FIG. 29 shows examples of controls and affecteds for all assays presented in FIG. 5. Typical results (‘Pyrograms’) of control and affected individuals for all assays (for X vs. Y and X vs. A assays males vs females are shown). The name of each assay is given on the left and the karyotypes on the top right comer of each panel. The PSM position is indicated by the grey numbers inside each box, which correspond to the chromosomes in which the paralogous sequence is located.

FIG. 30 shows the combined distribution for all assays presented in Example 5. Panel A shows the combined distributions of the autosomal assays. Panel B shows the distributions of the X vs. Y and X vs. A assays. The X-axes represent the percent of the ‘query’ chromosome, and the Y-axes the frequency of each class.

FIG. 31 shows the nucleotide sequences of relevant genes of the invention: A-ITSN, B-GABPA, C-NUFIP1, D-STK24, E-KIAA1328, F-WBP11, G-ARSD, H-TGIF2LX, I-TAF9L, J-JM5: K-SIM2: L-SIM1; M-CCT8, N-GABPA paralogue; O-CCT8 paralogue.

### DETAILED DESCRIPTION

The invention provides a method to detect the presence of chromosomal abnormalities by using paralogous genes as internal controls in an amplification reaction. The method is rapid, high-throughput, and amenable to semi-automated or fully automated analyses. In one aspect, the method comprises providing a pair of primers which can specifically hybridize to each of a set of paralogous genes under conditions used in amplification reactions, such as PCR. Paralogous genes are preferably on different chromosomes but may also be on the same chromosome (e.g., to detect loss or gain of different chromosome arms). By comparing the amount of amplified products generated, the relative dose of each gene can be determined and correlated with the relative dose of each chromosomal region and/or each chromosome, on which the gene is located.

Definitions

The following definitions are provided for specific terms which are used in the following written description.

As used herein the term “paralogous genes” refer to genes that have a common evolutionary origin but which have been duplicated over time in the human genome. Paralogous genes conserve gene structure (e.g., number and relative position of introns and exons, and preferably transcript length) as well as sequence. In one aspect, paralogous genes have at least about 80% identity, at least about 85% identity, at least about 90% identity, or at least about 95% identity over an amplifiable sequence region.

As used herein the term “amplifiable region” or an “amplifiable sequence region” refers to a single-stranded sequence defined at its 5′-most end by a first primer binding site and at its 3′-most end by a sequence complementary to a second primer binding site and which is capable of being amplified under amplification conditions upon binding of primers which specifically bind to the first and second primer binding sites in a double-stranded sequence comprising the amplifiable sequence region. Preferably, an amplifiable region is at least about 50 nucleotides, at least about 75 nucleotides, at least about 100 nucleotides, at least about 150 nucleotides, at least about 200 nucleotides, at least about 300 nucleotides, at least about 400 nucleotides, or at least about 500 nucleotides in length.

As used herein, a “primer binding site” refers to a sequence which is substantially complementary or fully complementary to a primer such that the primer specifically hybridizes to the binding site during the primer annealing phase of an amplification reaction.

As used herein, a “paralog set” or a “paralogous gene set” refers to at least two paralogous genes or paralogues.

As used herein a “chromosomal abnormality” or a “chromosomal imbalance” is a gain or loss of an entire chromosome or a region of a chromosome comprising one or more genes. Chromosomal abnormalities include monosomies, trisomies, polysomies, deletions and/or duplications of genes, including deletions and duplications caused by unbalanced translocations.

As used herein the term “high degree of sequence similarity” refers to sequence identity of at least about 80% over an amplifiable region.

As defined herein, “substantially equal amplification efficiencies” or “substantially the same amplification efficiencies” refers to amplification of first and second sequences provided in equal amounts to produce a less than about 10% difference in the amount of first and second amplification products.

As used herein, an “individual” refers to a fetus, newborn, child, or adult.

Identifying Paralogous Genes

Paralogous genes are duplicated genes which retain a high degree of sequence similarity dependent on both the time of duplication and selective functional restraints. Because of their high degree of sequence similarity, paralogous genes provide ideal templates for amplification reactions enabling a determination of the relative doses of the chromosome and/or chromosome region on which these genes are located.

Paralogous genes are genes that have a common evolutionary history but that have been replicated over time by either duplication or retrotransposition events. Duplication events generally result in two genes with a conserved gene structure, that is to say, they have similar patterns of intron—exon junctions. On the other hand paralogous genes generated by retrotransposition do not contain introns, and in most cases have been functionally inactivated through evolution, (not expressed) and are thus classed as pseudogenes. For both categories of paralogous genes there is a high degree of sequence conservation, however differences accumulate through mutations at a rate that is largely dependant on functional constraints.

In one aspect, the invention comprises identifying optimal paralogous gene sets for use in the method. For example, one can target certain areas of chromosomes where duplications events are known to have occurred using information available from the completed sequencing of the human genome (see, e.g., Venter et al., 2001, Science 291(5507): 1304-51; Lander et al., 2001, Nature 409(6822): 860-921). This may be done computationally by identifying a target gene of interest and searching a genomic sequence database or an expressed sequence database of sequences from the same species from which the target gene is derived to identify a sequence which comprises at least about 80% identity over an amplifiable sequence region. Preferably, the paralogous sequences comprise a substantially identical GC content (i.e., the sequences have less than about 5% and preferably, less than about 1% difference in GC content). Sequence search programs are well known in the art, and include, but are not limited to, BLAST (see, Altschul et al., 1990, J. Mol. Biol. 215: 403-410), FASTA, and SSAHA (see, e.g., Pearson, 1988, Proc. Natl. Acad. Sci. USA 85(5): 2444-2448; Lung et al., 1991, J. Mol. Biol. 221(4): 1367-1378). Further, methods of determining the significance of sequence alignments are known in the art and are described in Needleman and Wunsch, 1970, J. of Mol. Biol. 48: 444; Waterman et al., 1980, J. Mol. Biol. 147: 195-197; Karlin et al., 1990, Proc. Natl. Acad. Sci. USA 87: 2264-2268; and Dembo et al., 1994, Ann. Prob. 22: 2022-2039. While in one aspect, a single query sequence is searched against the database, in another aspect, a plurality of sequences are searched against the database (e.g., using the MEGABLAST program, accessible through NCBI). Multiple sequence alignments can be performed at a single time using programs known in the art, such as the ClustalW 1.6 (available at http://dot.imgen.bcm.tmc.edu:9331/multi-align/multi-align.html).

In a preferred embodiment, the genomic or expressed sequence database being searched comprises human sequences. Because of the completion of the human genome project (see, Venter et al., 2001, supra; Lander et al., 2001, supra), a computational search of a human sequence database will identify paralogous sets for multiple chromosome combinations. A number of human genomic sequence databases exist, including, but not limited to, the NCBI GenBank database (at http://www.ncbi.nlm.nih.gov/entrez/query.fcgi?db=Genome); the Celera Human Genome database (at http://www.celera.com); the Genetic Information Research Institute (GIRI) database (at http://www.girinst.org); TIGR Gene Indices (at http://www.tigr.org/tdb/tgi. shtml),and the like. Expressed sequence databases include, but are not limited to, the NCBI EST database, the LIFESEQ™ database (Incyte Pharmaceuticals, Palo Alto, Calif.), the random cDNA sequence database from Human Genome Sciences, and the EMEST8 database (EMBL, Heidelberg, Germany).

In one aspect, genes, or sets of genes, are randomly chosen as query sequences to identify paralogous gene sets. In another aspect, genes which have been identified as paralogous in the 5 literature are used as query sequences to search the database to identify regions of those genes which provide optimal amplifiable sequences (i.e., regions of the genes which have greater than about 80% identity over an amplifiable sequence region, and less than about a 1%-5% difference in GC content). Preferably, paralogous genes have conserved gene structures as well as conserved sequences; i.e., the number and relative positions of exons and introns are conserved 10 and preferably, transcripts generated from paralogous genes are substantially identical in size (i.e., have less than an about a 200 base pair difference in size, and preferably less than about a 100 base pair difference in size). Table 1 provides examples of non-limiting candidate paralogous gene sets which can be evaluated according to the method of the invention. Table 1A provides examples of non-limiting candidate paralogous gene sets, wherein one member of the set is located on chromosome 21, which can be evaluated according to the method of the invention. Table 1 B provides examples of additional non-limiting candidate paralogous gene sets which can be evaluated according to the method of the invention.

TABLE 1
Candidate Paralogous Genes

| Target region (Gene(s)) | Candidate Paralogous Region (Gene(s)) |
|---|---|
| Xq28 (SLC6A8) | 6p11.1 (DXS1357E) |
| Xq28 (ALD) | 2p11, 16p11, 22q11 (ALD-exons 7-10-paralogs) |
| Y (SRY) | 20p13(SOX22) |
| 1p33-34 (TALDOR) | 11p15 (TALDO) |
| 2q31 (Sp31) | 7p15 (Sp4); 12q13 (Sp1 gene) |
| 2 (COL3A1, COL5A2, COL6A3, COL4A3; | 12 (COL2A1, TUBAL1, GL1) |
| TUBA1, GL12) | |
| 2 (TGFA, SPTBN1) | 14 (TGFB3, SPTB) |
| 2p11 (ALD-exon 7-10 paralog) | Xq28 (ALD); 16p11 and 22q11 (ALD-exons 7-10 |
| | paralogs) |
| 3p21.3 (HYAL1, HYAL2, HYAL3) | 7q31.3 (HYAL4, SPAM1, HYALP1) |
| 3q22-q27 (CBLb) | 11q22-q24 (CBLa); 19 (band 13.2) (CBLc gene) |
| 3q29 (ERM) | 7p22 (ETV1); 17q12 (E1A-F) |
| 4 (FGR3, ADRA2L2, QDPR, GABRA2, GABRB1, | 5 (FGFR4, ADRA1, DHFR, GABRA1, PDGFRB, FGFA, |
| PDGFRA, FGF5, FGFB, F11, ANX3, ANX5) | F12, ANX6) |
| 5 (FGFR4, ADRA1, DHFR, GABRA1, PDGFRB, | 4 (FGR3, ADRA2L2, QDPR, GABRA2, GABRB1, |
| FGFA, F12, ANX6) | PDGFRA, FGF5, FGFB, F11, ANX3, ANX5) |
| 6p21.3 (COL11A2, NOTCH4, HSPA1A, HSPA1B, | 9q33-34 (COL5A1, NOTCH1, HSPA5, VARS1, C5; |
| HSPA1L, VARS2, C2, C4, PBX2, RXRB, | PBX3, RXRA, ORFX/RING3L) |
| NAT/RING3) | |
| 6q16.3-q21 (SIM1-confirmed paralog) | 21q22.2 (SIM2-confirmed paralog) |
| 7p22 (ETV1) | 3q29 (ERM); 17q12 (E1A-F) |
| 7q31.3 (HYAL4, SPAM1, HYALP1) | 3p21.3 (HYAL1, HYAL2, HYAL3) |
| 7 (MYH7) | 14 (MYH6) |
| 8q24.1-q24.2 (ANX13) | 10q22.3-q23.1 (ANX11) |
| 9q33-34 (COL5A1, NOTCH1, HSPA5, VARS1, C5, | 6p21.3 (COL11A2, NOTCH4, HSPA1A, HSPA1B, |
| PBX3, RXRA, ORFX/RING3L) | HSPA1L, VARS2, C2, C4, PBX2, RXRB, NAT/RING3) |
| 10p11 (ALD-exons 7-10-like) | Xq28 (ALD); 2p11 (ALD exons 7-10-like); 16p11 (ALD- |
| | exons 7-10-like); 22q11 (ALD-exons 7-10-like) |
| 10q22.3-q23.1 (ANX11) | 8q24.1-q24.2 (ANX13) |
| 11p15 (TALDO) | 1p33-34 (TALDOR) |
| 11q22-q24 (CBLa) | 19 (band 13.2) (CBLc gene); 3q22-q27(CBLb) |
| 11 (HRAS, IGF1; PTH) | 12 (KRAS2, IGF2, PTHLH) |
| 12 (COL2A1, TUBAL1, GL1) | 2 (COL3A1, COL5A2, COL6A3, COL4A3; TUBA1, |
| | GL12) |
| 12p12 (von Willebrand factor paralog) | 22q11 (von Willebrand factor paralog) |
| 14 (TGFB3, SPTB) | 2 (TGFA, SPTBN1) |
| 14 (MYH6) | 7 (MYH7) |
| 14q32.1 (GSC) | 22q11.21 (GSCL) |
| 15q24-q26 (TM6SF1) | 19p12-13.3 (TM6SF1) |
| 16p11.1 (DXS1357E) | Xq28 (SLC6A8) |
| 16p13.3(CREBBP, HMOX2) | 22q13 (adenovirus E1A-associated protein p300-CREBBP |
| | paralog); 22q12 (HMOX1-HMOX2 paralog) |
| 17q12 (E1A-F) | 3q29 (ERM); 7p22 (ETV1) |
| 17qtel (SYNGR2) | 22q13 (SYNGR1) |
| 19 (band 13.2) (CBLc gene) | 3q22-q27(CBLb); 11q22-q24 (CBLa) |
| 19p12-13.3 (TM6SF1) | 15q24-q26 (TM6SF1) |
| 20p13 (SOX22) | Y (SRY) |
| 21q22.2 (SIM2-confirmed paralog) | 6q16.3-q21 (SIM1-confirmed paralog) |
| 22q13 (SYNGR1) | 17qtel (SYNGR2) |
| 22q11 (von Willebrand factor paralog) | 12p12 (von Willebrand factor paralog) |
| 22q11.21 (GSCL) | 14q32.1 (GSC) |

TABLE 1A
Chromosome 21 Gene and its Paralogous Copy.

| | | Paralogous | |
|---|---|---|---|
| Chromosome 21 gene | Position | Gene position | Class |
| GABPA | 21q22.1 | HC 7 | pseudogene |
| CCT8 | 21q22.2 | HC 1 | pseudogene |
| C21ORF19 | 21q22.2 | HC 2 | Expressed |
| | | | gene |
| DSCR3 | 21q22.2 | HC 2 | pseudogene |
| C21Orf6 | 21q22.2 | HC 4 | pseudogene |
| SIM2 | 21q22.2 | HC 6 | Expressed |
| | | | gene |
| WRB1 | 21q22.2 | HC 12 | Expressed |
| | | | gene |
| KIAAO958 | 21q22.3 | HC 7 | pseudogene |
| TTC3 | 21q22.3 | HC X | pseudogene |
| ITSN1 | 21q22.2 | HC 5 | Expressed |
| | | | gene |

TABLE 1B
Additional Candidate Paralogous Genes

| | |
|---|---|
| Trisomy 13 | Trisomy 18 |

| Gene | Paralogous target | Gene | Paralogous target |
|---|---|---|---|
| RAP2A | HC3 pseudogene | ACAA2 | HC2 Pseudogene |
| CDK8 | HC2 Pseudogene | ME2 | HC9 Pseudogene |

Paralogous gene sets useful according to the invention include but are not limited to the following, all incorporated by reference in their entirety: GABPA (Accession No.: NM_{—}002040, NT_{—}011512, XM009709, AP001694, X84366) and the GABPA paralogue (Accession No.: LOC154840); CCT8 (Accession No.: NM_{—}006585, NT_{—}011512, AL163249, G09444) and the CCT8 paralogue (Accession No.: LOC149003); RAP2A (Accession No.: NM_{—}021033) and the RAP2A paralogue (Accession No.: NM_{—}002886); ME2 (Accession No.: NM_{—}002396) and an ME2 paralogue ; CDK8 (Accession No.: NM_{—}001260) and a CDK8 paralogue (Accession No.: LOC129359); ACAA2 (Accession No.: NM_{—}006111) and an ACAA2 paralogue; DSCR3 (Accession Nos.: NT_{—}011512, NM_{—}006052, AP001728) and a DSCR3 paralogue; C21orf19 (Accession Nos.: NM_{—}015955, NT_{—}005367, AF363446, AP001725) and a C21orf19 paralogue; KIAA0958 (Accession Nos.: NT_{—}011514, NM_{—}015227, AL163301, AB023175) and a KIAA0958 paralogue; TTC3 (Accession Nos.: NM_{—}003316, NT_{—}011512, AP001727, AP001728) and a TTC3 paralogue; ITSN1 (Accession Nos.: NT_{—}011512, NM_{—}003024, XM_{—}048621) and a ITSN1 paralogue; NUFIP1 (Accession No.: NM_{—}012345); STK24 (Accession No.: NM_{—}003576); KIAA1328 (Accession No.:ABO37749); WBP11 (Accession No.:NM_{—}016312); ARSD (Accession No.:NM_{—}009589); TGIF2LX (Accession No.:NM_{—}138960); TAF9L (Accession No.: NM_{—}015975); JM5 (Accession No.: NM_{—}007075).

Additional paralogous gene sets which can be used as query sequences include the HOX genes. Related HOX genes and their chromosomal locations are described in Popovici et al., 2001, FEBS Letters 491: 237-242. Candidate paralogs for genes in chromosomes 1, 2, 7, 11, 12, 14, 17, and 19 are described further in Lundin, 1993, Genomics 16: 1-19. The entireties of these references are incorporated by reference herein.

In still another aspect, query sequences are identified by targeting regions of the human genome which are duplicated (e.g., as determined by analysis of the completed human genome sequence) and these sequences are used to search database(s) of human genomic sequences to identify sequences at least 80% identical over an amplifiable sequence region.

In a further aspect, a clustering program is used to group expressed sequences in a database which share consensus sequences comprising at least about 80% identity over an anplifiable sequence region, to identify suitable paralogs. Sequence clustering programs are known in the art (see, e.g., Guan et al., 1998, Bioinformatics 14(9): 783-8; Miller et al., Comput. Appl. Biosci. 13(1): 81-7; and Parsons, 1995, Comput. Appl. Biosci. 11(6): 603-13, the entireties of which are incorporated by reference herein).

While computational methods of identifying suitable paralog sets are preferred, any method of detecting sequences which are capable of significant base pairing can be used and are encompassed within the scope of the invention. For example, paralogous gene sets can be identified using a combination of hybridization-based methods and computational methods. In this aspect, a target chromosome region can be identified and a nucleic acid probe corresponding to that region can be selected (e.g., from a BAC library, YAC library, cosmid library, cDNA library, and the like) to be used in in situ hybridization assays (FISH or ISH assays) to identify probes which hybridize to multiple chromosomes (preferably fewer than about 5). The specificity of hybridization can be verified by hybridizing a target probe to flow sorted chromosomes thought to contain the paralogous gene(s), to chromosome-specific libraries and/or to somatic cell hybrids comprising test chromosome(s) of interest (see, e.g., Horvath, et al., 2000, Genome Research 10: 839-852). Successively smaller probe fragments can be used to narrow down a region of interest thought to contain paralogous genes and these fragments can be sequenced to identify optimal paralogous gene sets.

Although in one aspect, paralogous genes are used as amplification templates in methods of the invention, any paralogous sequence which comprises sufficient sequence identity to provide substantially identical amplification templates having fewer than about 20% nucleotide differences over an amplifiable region is contemplated. For example, pseudogenes can be included in paralog sets as can non-expressed sequences, provided there is sufficient identity between sequences in each set.

Sources of Nucleic Acids

In one aspect, the method according to the invention is used in prenatal testing to assess the risk of a child being born with a chromosomal abnormality. For these types of assays, samples of DNA are obtained by procedures such as amniocentesis (e.g., Barter, Am. J Obstet. Gynecol. 99: 795-805; U.S. Pat. No. 5,048,530), chorionic villus sampling (e.g., Imamura et al., 1996, Prenat. Diagn. 16(3): 259-61), or by maternal peripheral blood sampling (e.g., Iverson et al., 1981, Prenat. Diagn. 9: 31-48; U.S. Pat. No. 6,210,574). Fetal cells also can be obtained by cordocentesis or percutaneous umbilical blood sampling, although this technique is technically difficult and not widely available (see Erbe, 1994, Scientific American Medicine 2, section 9, chapter IV, Scientific American Press, New York, pp 41-42). Preferably, DNA is isolated from the fetal cell sample and purified using techniques known in the art (see, e.g., Maniatis et al., In Molecular Cloning, Cold Spring Harbor, N.Y., 1982)).

However, in another aspect, cells are obtained from adults or children (e.g., from patients suspected of having cancer). The invention also encompasses fetal cells that are purified from maternal blood. Cells can be obtained from blood samples or from a site of cancer growth (e.g., a tumor or biopsy sample) and isolated and purified as described above, for subsequent amplification.

Amplification Conditions

Having identified a paralogous gene set comprising a target gene whose dosage is to be determined and a reference gene having a known dosage, primer pairs are selected to produce amplification products from each gene which are similar or identical in size. In one aspect, the amplification products generated from each paralogous gene differ in length by no greater than about 0-75 nucleotides, and preferably, by no greater than about 0 to 25 nucleotides. Primers for amplification are readily synthesized using standard techniques (see, e.g., U.S. Pat. Nos. 4,458,066; 4,415,732; and Molecular Protocols Online at http://www.protocol-online.net/molbio/PCR/pcr_primer.htm). Preferably, primers are from about 6-50 nucleotides in length and amplification products are at least about 50 nucleotides in length.

Although in a preferred method, primers are unlabeled, in some aspects, primers are labeled using methods well known in the art, such as by the direct or indirect attachment of radioactive labels, fluorescent labels, electron dense moieties, and the like. Primers can also be coupled to capture molecules (e.g., members of a binding pair) when it is desirable to capture amplified products on solid supports (see, e.g., WO 99/14376).

Amplification of paralogous genes can be performed using any method known in the art, including, but not limited to, PCR (Innis et al., 1990, PCR Protocols. A Guide to Methods and Application, Academic Press, Inc. San Diego), Ligase Chain Reaction (LCR) (Wu and Wallace, 1989, Genomics 4: 560, Landegren, et al., 1988, Science 241: 1077), Self-Sustained Sequence Replication (3SR) (Guatelli et al., 1990, Proc. Natl. Acad. Sci. USA 87:1874-1878), and the like. However, preferably, genes are amplified by PCR using standard conditions (see, for example, as described in U.S. Pat. Nos. 4,683,195; U.S. Pat. No. 4,800,159; U.S. Pat. No. 4,683,202; and U.S. Pat. No. 4,889,818).

In one aspect, amplified DNA is immobilized to facilitate subsequent quantitation. For example, primers coupled to a first member of a binding pair can be attached to a support on which is bound a second member of the binding pair capable of specifically binding to the first member. Suitable binding pairs include, but are not limited to, avidin: biotin, antigen: antibody pairs; reactive pairs of chemical groups, and the like. In one aspect, primers are coupled to the support prior to amplification and immobilization of amplification products occurs during the amplification process itself. Alternatively, amplification products can be immobilized after amplification. Solid supports can be any known and used in the art for solid phase assays (e.g., particles, beads, magnetic or paramagnetic particles or beads, dipsticks, capillaries, microchips, glass slides, and the like) (see, e.g., as described in U.S. Pat. No. 4,654,267). Preferably, solid supports are in the form of microtiter wells (e.g., 96 well plates) to facilitate automation of subsequent quantitation steps.

Quantitating Gene Dose

Quantitation of individual paralogous genes can be performed by any method known in the art which can detect single nucleotide differences. Suitable assays include, but are not limited to, real time PCR (TAQMAN®), allele-specific hybridization-based assays (see, e.g., U.S. Pat. No. 6,207,373); RFLP analysis (e.g., where a nucleotide difference creates or destroys a restriction site), single nucleotide primer extension-based assays (see, e.g., U.S. Pat. No. 6,221,592); sequencing-based assays (see, e.g., U.S. Pat. No. 6,221,592), and the like.

In a preferred embodiment of the invention, quantitation is performed using a pyrosequencing™ method (see, e.g., U.S. Pat. No. 6,210,891 and U.S. Pat. No. 6,197,505, the entireties of which are incorporated by reference). In this method, the amplification products of the paralogous genes are rendered single-stranded and incubated with a sequencing primer comprising a sequence which specifically hybridizes to the same sequence in each paralogous gene in the presence of DNA polymerase, ATP sulfurylase, luciferase, apyrase, adenosine 5′ phosphosulfate (APS), and luciferin. Suitable polymerases include, but are not limited to, T7 polymerase, (exo⁻) Klenow polymerase, Sequenase® Ver. 2.0 (USB U.S.A.), Taq™ polymerase, and the like. The first of four deoxynucleotide triphosphates (dNTPs) is added (with deoxyadenosine α-thio-triphosphate being used rather than dATP) and, if incorporated into the primer through primer extension, pyrophosphate (PPi) is released in an amount which is equimolar to the amount of the incorporated nucleotide. PPi is then quantitatively converted to ATP by ATP sulfurylase in the presence of APS. The release of ATP into the sample causes luciferin to be converted to oxyluciferin by luciferase in a reaction which generates light in amounts proportional to the amount of ATP. The released light can be detected by a charge-coupled device (CCD) and measured as a peak on a pyrogram display (e.g., in a Pyrosequencing™ PSQ 96 DNA/SNP analyzer available from Pyrosequencing™, Inc., Westborough, Mass. 01581). The apyrase degrades the unincorporated dNTPs and when degradation is complete (e.g., when no more light is detected), another dNTP is added. Addition of dNTPs is performed one at a time and the nucleotide sequence is determined from the signal peak. The presence of two contiguous bases comprising identical nucleotides is detectable as a proportionally larger signal peak.

In a currently preferred embodiment, chromosome dosage in a nucleic acid sample is evaluated by using a pyrosequencing™ method to determine the ratio of sequence differences in paralogous sequences which differ at at least one nucleotide position. For example, in one aspect, two paralogous sequences from two paralogous genes, each on different chromosomes, are sequenced and the ratios of different nucleotide bases at positions of sequence differences in the two paralogs are determined. A 1:1 ratio of different nucleotide bases at a position where the two sequences differ indicates a 1:1 ratio of chromosomes. However, a difference from a 1:1 ratio indicates the presence of a chromosomal imbalance in the sample. For example, a ratio of 3:2 would indicate the presence of a trisomy. Paralogous sequences on the same chromosome can also be evaluated in this way (for example, to determine the loss or gain of a particular chromosome arm).

Using a Pyrosequencing™ PSQ 96 DNA/SNP analyzer, 96 samples can be analyzed simultaneously in less than 30 minutes. By using sequencing primers which hybridize adjacent to the portion of the paralog sequence which is unique to each of the paralogs, it can be possible to distinguish between the paralogs after only one or a few rounds of dNTP incorporation (i.e., performing minisequencing). The analysis does not require gel electrophoresis or any further sample processing since the output from the Pyrosequencer provides a direct quantitative ratio enabling the user to infer the genotype and hence phenotype of the individual from whom the sample is obtained. By using a paralogous gene as a natural internal control, the amount of variability from sample handling is reduced. Further, no radioactivity or labeling is required.

Diagnostic Applications

Amplification of paralogous gene sets can be used to determine an individual's risk of having a chromosomal abnormality. Using a paralogous gene set including a target gene from a chromosome region of interest and a reference gene, preferably on a different chromosome, the ratio of the genes is determined as described above. Deviations from a 1:1 ratio of target to reference gene indicates an individual at risk for a chromosomal abnormality. Examples of chromosome abnormalities which can be evaluated using the method according to the invention are provided in Table 2 below.

TABLE 2
Chromosome Abnormalities and Disease

| Chromosome Abnormality | | Disease Association |
|---|---|---|
| X, | XO | Turner's Syndrome |
| Y | XXY | Klinefelter syndrome |
| | XYY | Double Y syndrome |
| | XXX | Trisomy X syndrome |
| | XXXX | Four X syndrome |
| | Xp21 deletion | Duchenne's/Becker syndrome, congenital adrenal hypoplasia, |
| | | chronic granulomatus disease |
| | Xp22 deletion | steroid sulfatase deficiency |
| | Xq26 deletion | X-linked lymphproliferative disease |
| 1 | 1p-(somatic) | neuroblastoma |
| | monosomy | |
| | trisomy | |
| 2 | monosomy | |
| | trisomy 2q | growth retardation, developmental and mental delay, and minor |
| | | physical abnormalities |
| 3 | monosomy | |
| | trisomy (somatic) | non-Hodgkin's lymphoma |
| 4 | monosomy | |
| | trsiomy (somatic) | Acute non lymphocytic leukaemia (ANLL) |
| 5 | 5p- | Cri du chat; Lejeune syndrome |
| | 5q-(somatic) | myelodysplastic syndrome |
| | monosomy | |
| | trisomy | |
| 6 | monosomy | |
| | trisomy (somatic) | clear-cell sarcoma |
| | 7q11.23 deletion | William's syndrome |
| | monosomy | monosomy 7 syndrome of childhood; somatic: renal cortical |
| | | adenomas; myelodysplastic syndrome |
| | trisomy | |
| 8 | 8q24.1 deletion | Langer-Giedon syndrome |
| 8 | monosomy | |
| | trisomy | myelodysplastic syndrome; Warkany syndrome; somatic: chronic |
| | | myelogenous leukemia |
| 9 | monosomy 9p | Alfi's syndrome |
| | monosomy | |
| | 9p partial trisomy | Rethore syndrome |
| | trisomy | complete trisomy 9 syndrome; mosaic trisomy 9 syndrome |
| 10 | monosomy | |
| | trisomy (somatic) | ALL or ANLL |
| 11 | 11p- | Aniridia; Wilms tumor |
| | 11q- | Jacobson Syndrome |
| | monosomy (somatic) | myeloid lineages affected (ANLL, MDS) |
| | trisomy | |
| 12 | monosomy | |
| | trisomy (somatic) | CLL, Juvenile granulosa cell tumor (JGCT) |
| 13 | 13q- | 13q-syndrome; Orbeli syndrome |
| | 13q14 deletion | retinoblastoma |
| | monosomy | |
| | trisomy | Patau's syndrome |
| 14 | monsomy | |
| | trisomy (somatic) | myeloid disorders (MDS, ANLL, atypical CML) |
| 15 | 15q11-q13 deletion | Prader-Willi, Angelman's syndrome |
| | monosomy | |
| | trisomy (somatic) | myeloid and lymphoid lineages affected, e.g., MDS, ANLL, ALL, |
| | | CLL) |
| 16 | 16q13.3 deletion | Rubenstein-Taybi |
| | monosomy | |
| | trisomy (somatic) | papillary renal cell carcinomas (malignant) |
| 17 | 17p-(somatic) | 17p syndrome in myeloid malignancies |
| | 17q11.2 deletion | Smith-Magenis |
| | 17q13.3 | Miller-Dieker |
| | monosomy | |
| | trisomy (somatic) | renal cortical adenomas |
| | 17p11.2-12 trisomy | Charcot-Marie Tooth Syndrome type 1; HNPP |
| 18 | 18p- | 18p partial monosomy syndrome or Grouchy Lamy Thieffry |
| | | syndrome |
| | 18q- | Grouchy Lamy Salmon Landry Syndrome |
| | monosomy | |
| | trisomy | Edwards Syndrome |
| 19 | monosomy | |
| | trisomy | |
| 20 | 20p- | trisomy 20p syndrome |
| | 20p11.2-12 deletion | Alagille |
| | 20q- | somatic: MDS, ANLL, polycythemia vera, chronic neutrophilic |
| | | leukemia |
| | monosomy | |
| | trisomy (somatic) | papillary renal cell carcinomas (malignant) |
| 21 | monosomy | |
| | trisomy | Down's syndrome |
| 22 | 22q11.2 deletion | DiGeorge's syndrome, velocardiofacial syndrome, conotruncal |
| | | anomaly face syndrome, autosomal dominant Opitz G/BBB |
| | | syndrome, Caylor cardiofacial syndrome |
| | monosomy | |
| | trisomy | complete trisomy 22 syndrome |

Generally, evaluation of chromosome dosage is performed in conjunction with other assessments, such as clinical evaluations of patient symptoms. For example, prenatal evaluation may be particularly appropriate where parents have a history of spontaneous abortions, still births and neonatal death, or where advanced maternal age, abnormal maternal sera results, and in patients with a family history of chromosomal abnormalities. Postnatal testing may be appropriate where there are multiple congenital abnormalities, clinical manifestations consistent with known chromosomal syndromes, unexplained mental retardation, primary and secondary amenorrhea, infertility, and the like.

The method is premised on the assumption that the likelihood that two chromosomes will be altered in dose at the same time will be negligible (i.e., that the test and reference chromosome comprising the test and reference paralogous sequence, respectively, are not likely to be monosomic or trisomic at the same time). Further, assays are generally performed using samples comprising normal complements of chromosomes as controls. However, in one aspect, multiple sets of paralogous genes, each set from different pairs of chromosomes, are used to increase the sensitivity of the assay. In another aspect, for example, in postnatal testing, amplification of an autosomal paralogous gene set is performed at the same time as amplification of an X chromosome sequence since X chromosome dosage can generally be verified by phenotype. In still another aspect, a hierarchical testing scheme can be used. For example, a positive result for trisomy 21 using the method according to the invention could be followed by a different test to confirm altered gene dosage (e.g., such as by assaying for increases in PKFL-CH21 activity and an absence of M4-type phosphofructokinase activity; see, e.g., as described in Vora, 1981, Blood 57: 724-731), while samples showing a negative result would generally not be further analyzed. Thus, the method according to the invention would provide a high throughput assay to identify rare cases of chromosome abnormalities which could be complemented with lower throughput assays to confirm positive results.

Similarly, the assumption that loss or gain of a paralogous gene reflects loss or gain of a chromosome versus a chromosome arm versus a chromosome band versus only the paralogous gene itself, can be validated by complementing the method according to the invention with additional tests, for example, by using multiple sets of paralogous genes on the same chromosome, each set corresponding to a different chromosome region.

The invention will now be further illustrated with reference to the following example. It will be appreciated that what follows is by way of example only and that modifications to detail may be made while still falling within the scope of the invention.

### EXAMPLES

### Example 1

The following examples describe a PCR based method for detecting a chromosomal imbalance, for example, trisomy 21 by coamplifying, with a single set of primers, paralogous genes present in different chromosomes.

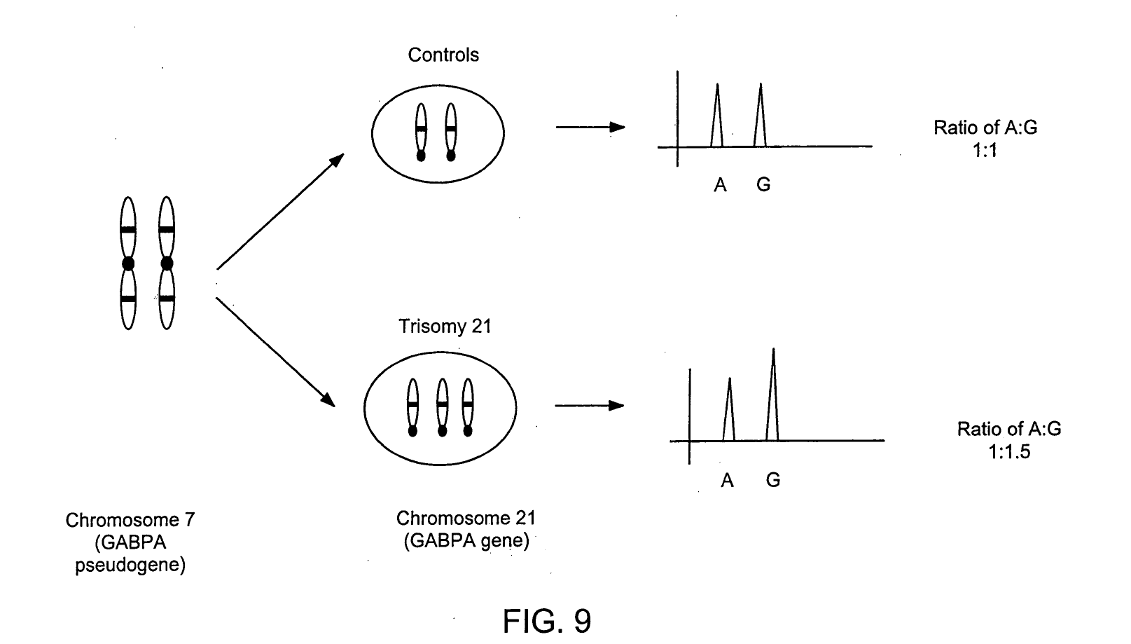

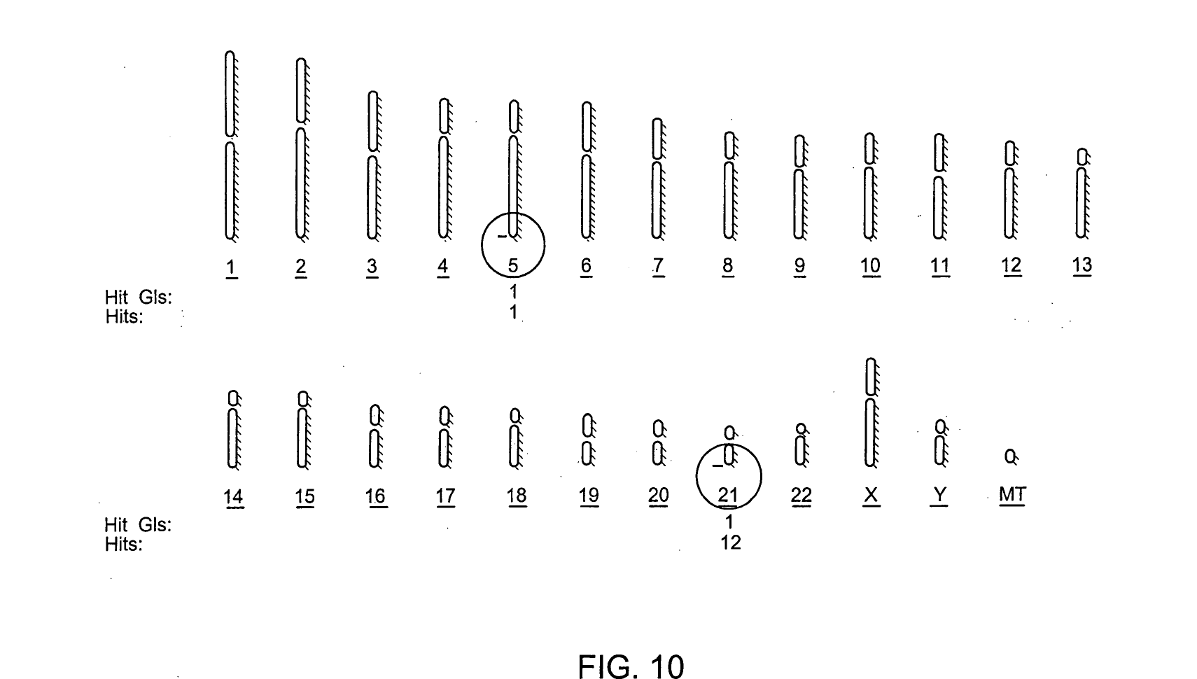

The rationale for using paralogous genes is that since they are of almost identical size and sequence composition, they will PCR amplify with equal efficiency using a single pair of primers. Single nucleotide differences between the two sequences are identified, and the relative amounts of each allele, each of which represents a chromosome, are quantified (see FIG. 9). Since the pyrosequencing method is highly quantitative one can accurately assay the ratio between the chromosomes.

For detecting Trisomy 21, the method involves the following steps:
a. Identification of suitable candidates for co-amplification (paralogous genes);
b. Design of multiple assays for co-amplification of paralogous sequences between human chromosome 21 and other chromosomes;
c. Testing the assays using a panel of Trisomy 21 and control DNA samples;
d. Testing the robustness of the method on a suitably large retrospective sample.

Analogous steps are used to detect any chromosomal imbalance according to the invention.

Identification of Paralogous Genes

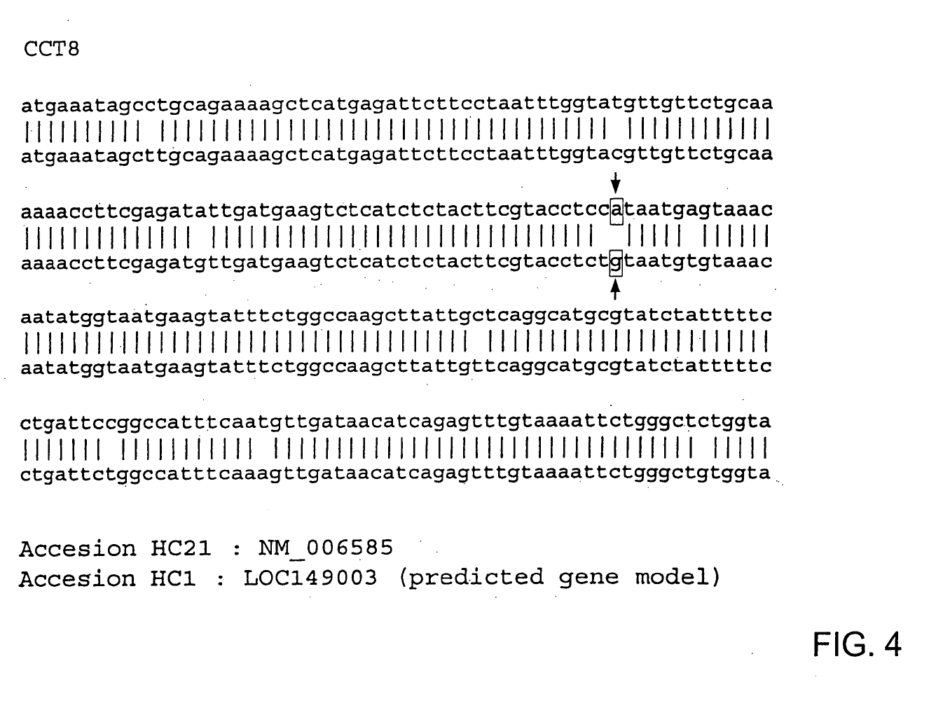

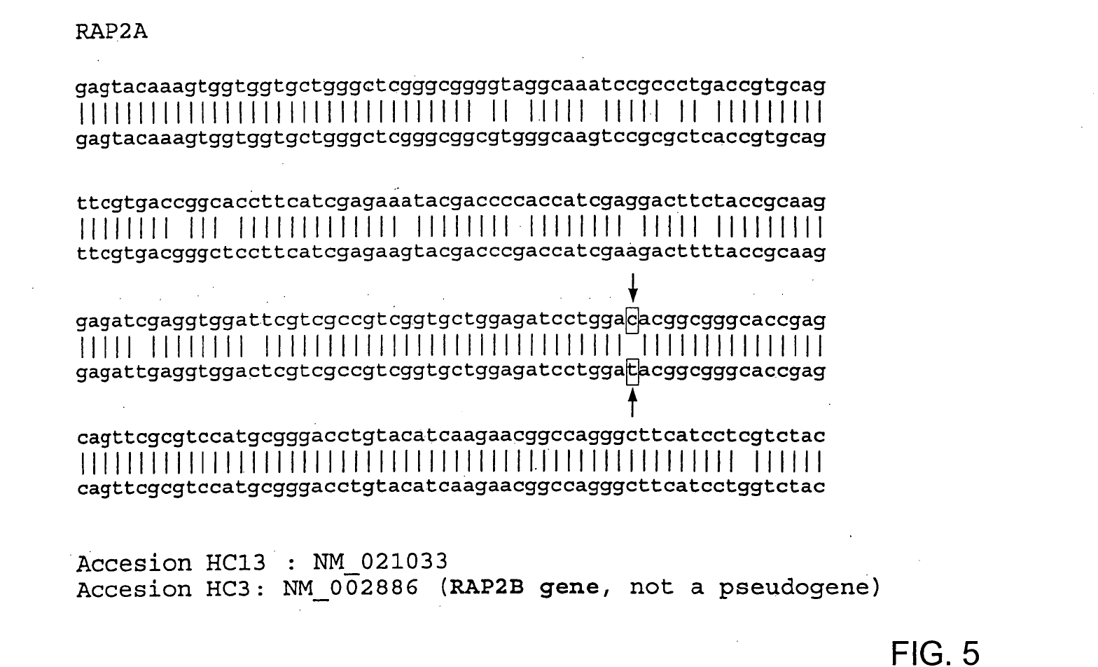

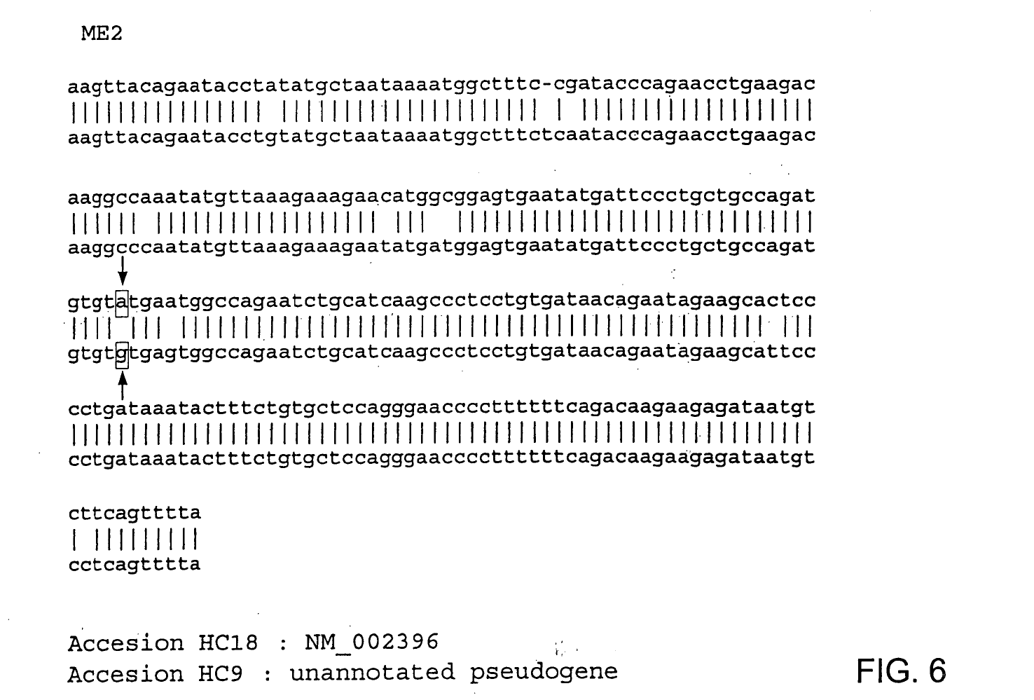

In order to identify paralogous sequences between chromosome 21 and the rest of the genome all chromosome 21 genes and pseudogenes (cDNA sequence) located between the 21q 22.1 region and the telomere were blasted against (compared with) the non redundant human genome database (http://www.ncbi.nlm.nih.gov/genome/seq/HsBlast.html), (FIG. 4) as this region is present in three copies in all individuals reported with Down syndrome.

From this, 10 potential candidate pairs which could serve as suitable targets for co-amplification were identified (table 1A).

Most of these pairs are formed by a functional gene and an unspliced pseudogene suggesting that the most common origin of these paralogous copies is retrotransposition rather than ancient chromosomal duplications.

Samples

In order to perform the retrospective validation studies for the two optimized tests, 400 DNA samples (200 DNAs from trisomic individuals and 200 control DNAs) were used. These samples were collected with informed consent by the Division of Medical Genetics, University of Geneva over the past 15 years. The samples were extracted at different periods with presumably different methods, hence the quality of these DNAs is not expected to be uniform.

Concerning the use of these samples for the development of a Diagnostic method, permission was granted by the local ethics committee for this specific use.

The invention provides for methods wherein the samples used are either freshly prepared or stored, for example at 4° C., preferably frozen at at least −20° C., and more preferably frozen in liquid nitrogen.

Assay Design

Using the results summarized in table 1A, a first round of assays were designed and performed.

A critical aspect for assay development is to choose regions of very high sequence conservation (between 70 and 95% and preferably between 85-95%) that are contained within the same exon in both genes (this is necessary so that both amplicons are of equal size), and that comply with the following conditions:
1. There are long stretches of perfect sequence conservation from which compatible primers can be designed.
2. One or more single nucleotide differences are present within the amplimers which are surrounded by perfectly homologous sequence so that a suitable sequencing primer can be designed.

Using these criteria assays were developed for the GABPA gene and the CCT8 gene.

### Example 2

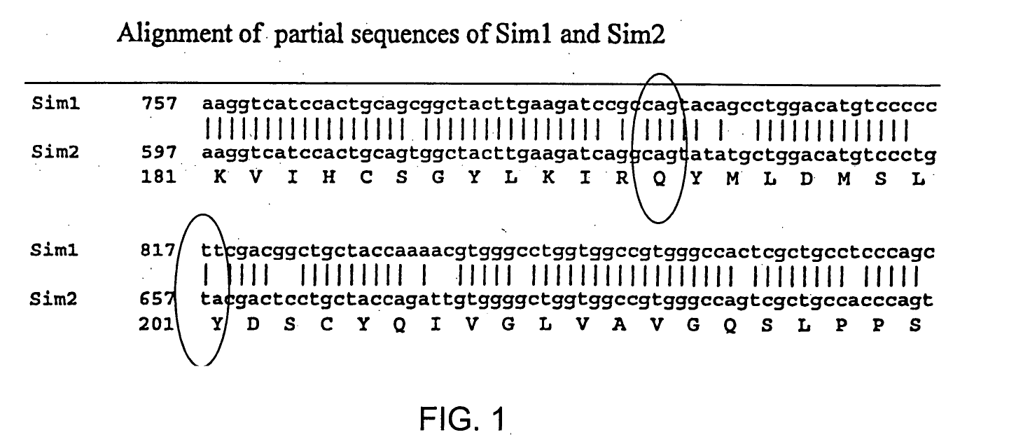

Trisomy 21 is detected by providing a sample comprising at least one cell from a patient (e.g., a fetus) and extracting DNA from the cell(s) using standard techniques. The sample is incubated with a single pair of primers which will specifically anneal to both SIM2 (GenBank accession nos. U80456, U80457, and AB003185) and SIM1 genes (GenBank accession no. U70212), paralogous genes located on chromosome 21 and chromosome 6, respectively, under standard annealing conditions used in PCR. Alignment of partial sequences of SIM2 and SIMI1 is shown in FIG. 1.

Using primer sequences SIMAF (GCAGTGGCTACTTGAAGAT) and SIMAR (TCTCGGTGATGGCACTGG), the sample is subjected to PCR conditions. For example, providing 5.0 μl of amplification buffer, 200 μM dNTPs, 3 mM MgCl₂, 50 ng DNA, and 5 Units of Taq polymerase, 35 cycles of touchdown PCR (e.g., 94° C. for 30 seconds; 63-58° C. for 30 seconds; and 72° C. for 10 seconds) generates suitable amounts of amplification products for subsequent detection of sequence differences between the two paralogs.

The amount of amplified products corresponding to SIM1 and SIM2 is determined by assaying for single nucleotide differences which distinguish the two genes (see circled sequences in FIG. 1). Preferably this is done by a pyrosequencing™ method, using sequencing primer SIMAS (GTGGGGCTGGTGGCCGTG). The expected sequence obtained from the pyrosequencing™ reaction is GGCCA[C/G]TCGCTGCC; the brackets and bold highlighting indicating the position of a sequence difference between the two sequences.

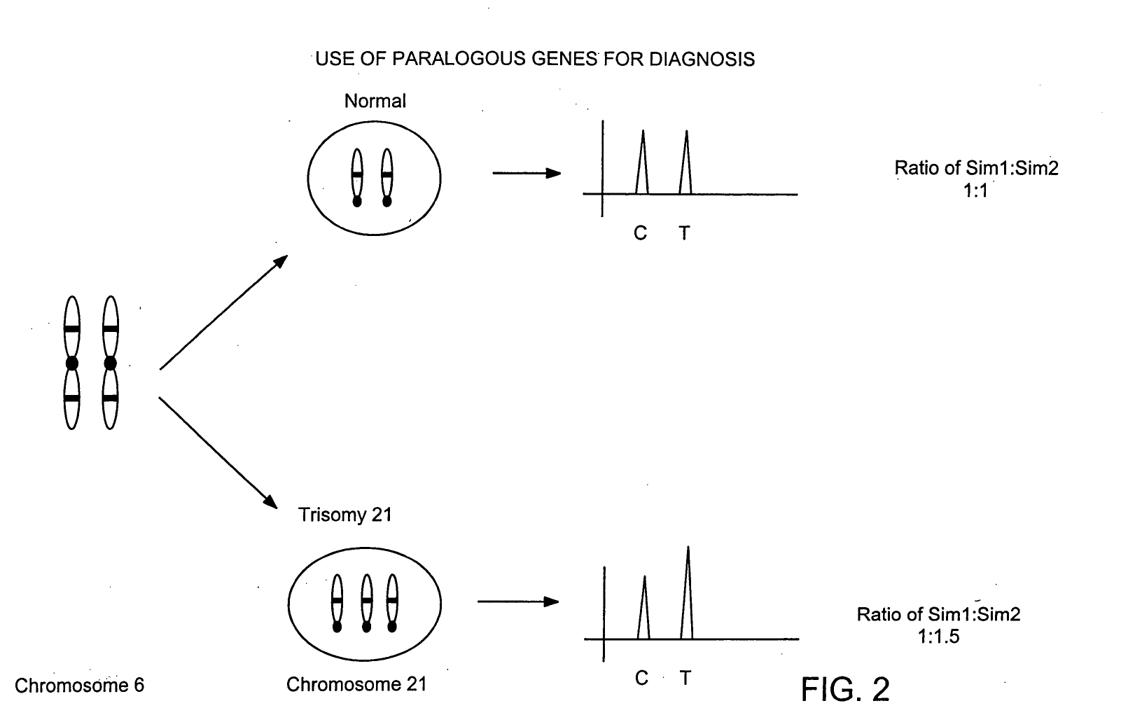

The allele ratio of SIM2:SIM1 is determined by comparing the ratio of one base with respect to another at the site of a nucleotide difference between the two paralogs. As can be seen in FIG. 2, the ratio of such a base is 1:1.5 in a Down syndrome individual and 1:1 in a normal individual.

### Example 3

The following example describes a method for detecting Trisomy 21 according to the method of the invention, wherein one member of the paralogous gene pair is GABPA.

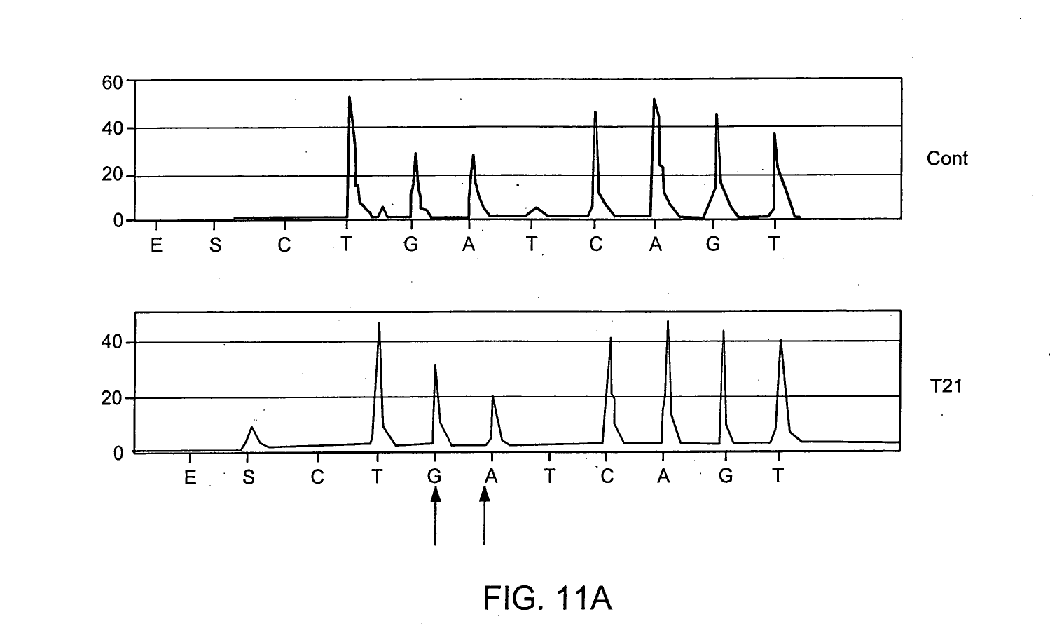

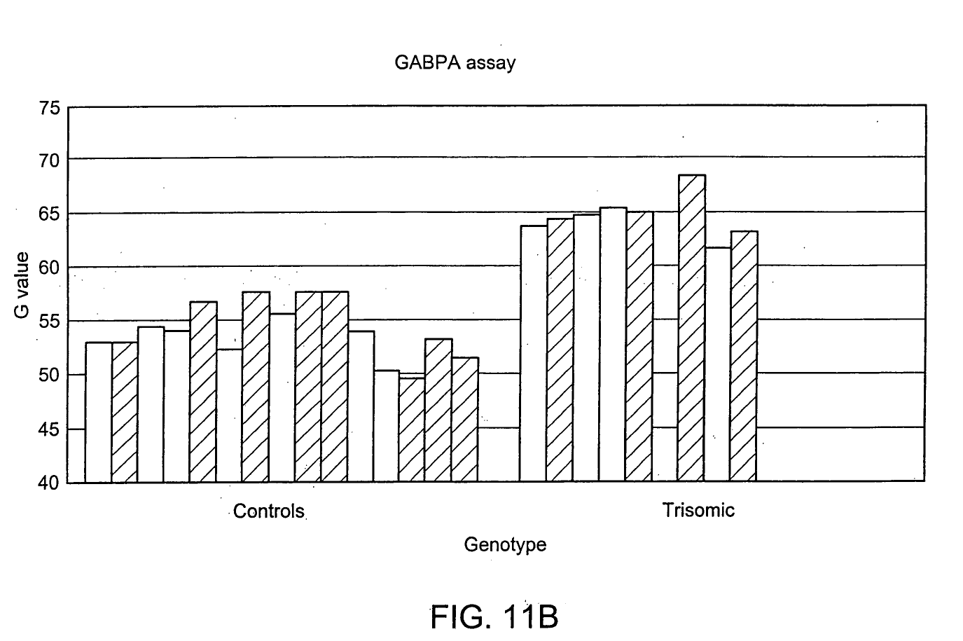

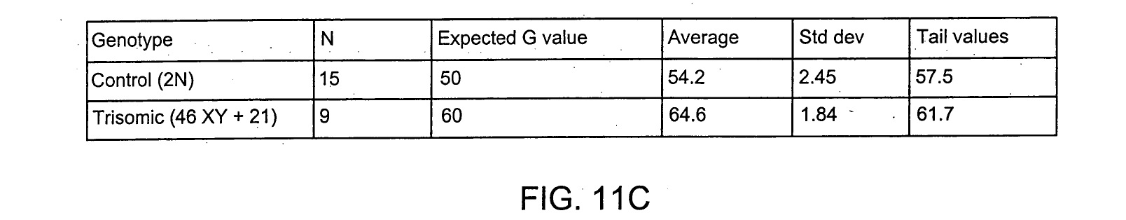

Trisomy 21 is detected by providing a sample comprising at least one cell from a patient (e.g., a fetus) and extracting DNA from the cell(s) using standard techniques. The results of a pilot experiment are presented in FIG. 11. Following the performance of the pilot experiments, the assays were further optimized by identifying sets of primers with a higher efficiency of amplification and a smaller intra and inter sample variation. The details of the optimized assay for detection of trisomy 21 are provided below.

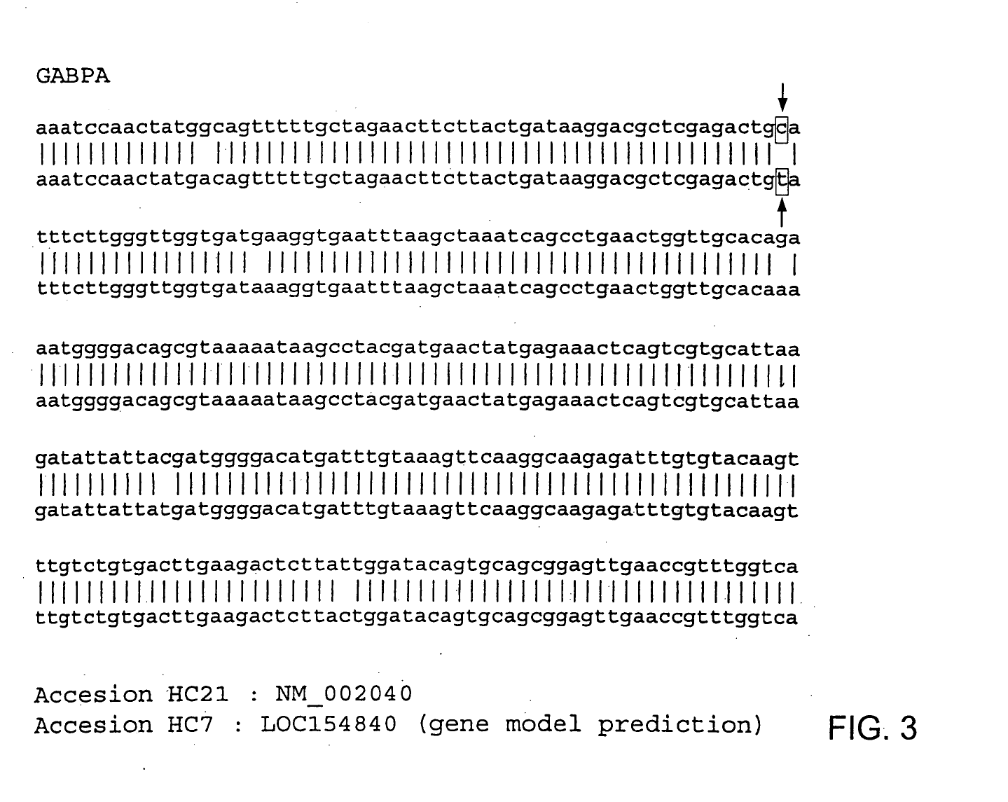

Four Hundred DNA samples (200 trisomic and 200 control samples) were incubated with a single pair of primers which will specifically anneal to both a GABPA gene paralogue (GenBank accession nos. LOC154840) and GABPA genes (GenBank accession no. NM_{—}002040), paralogous genes located on chromosome 7 and chromosome 21, respectively, under standard annealing conditions used in PCR. Alignment of sequences of the GABPA gene paralogue and GABPA is shown in FIG. 3.

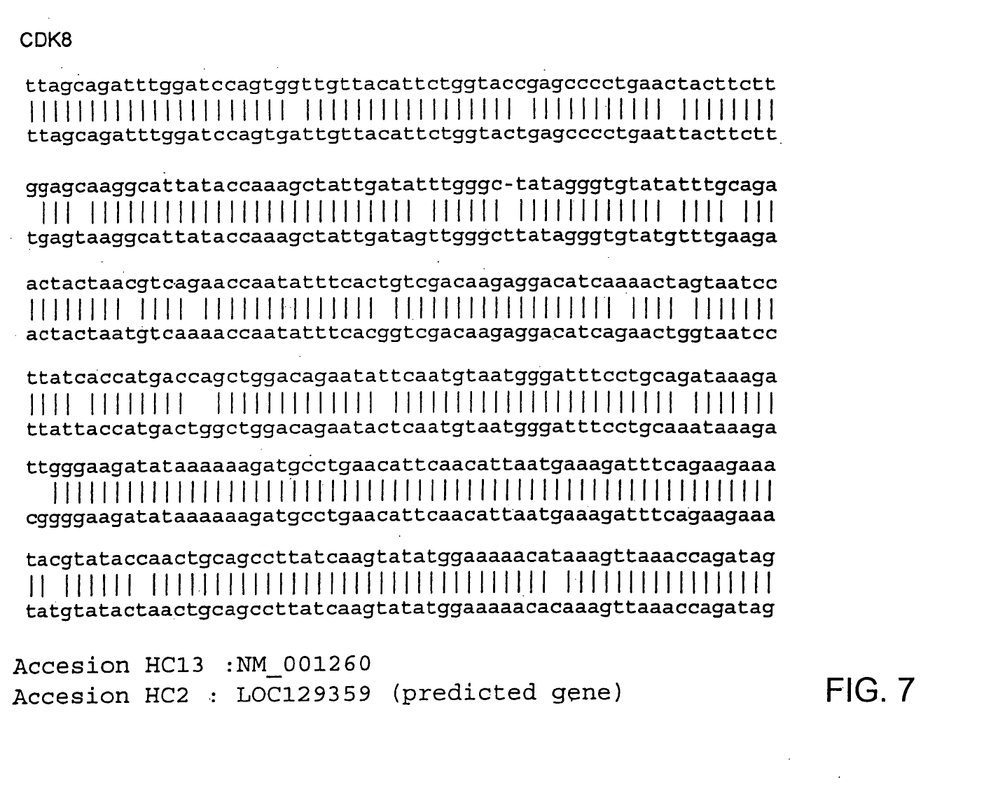

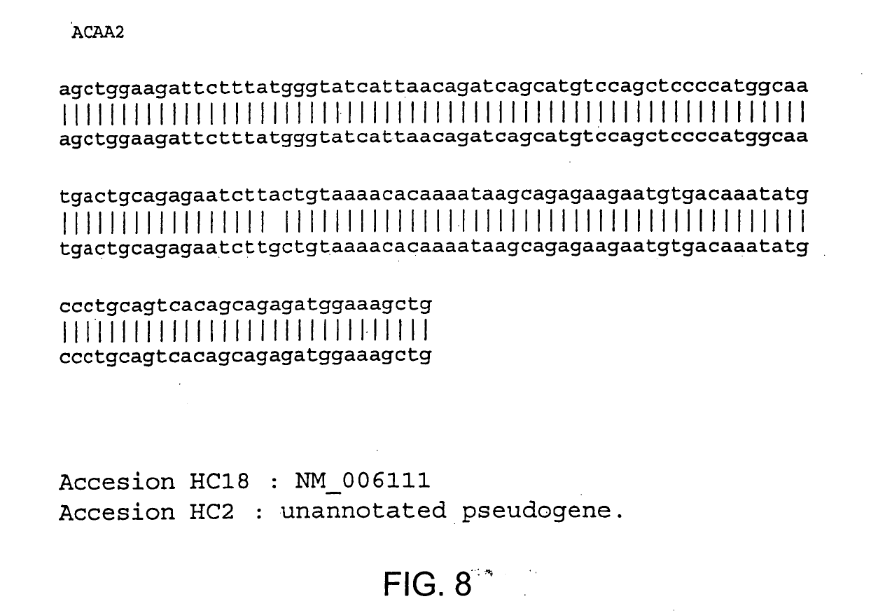

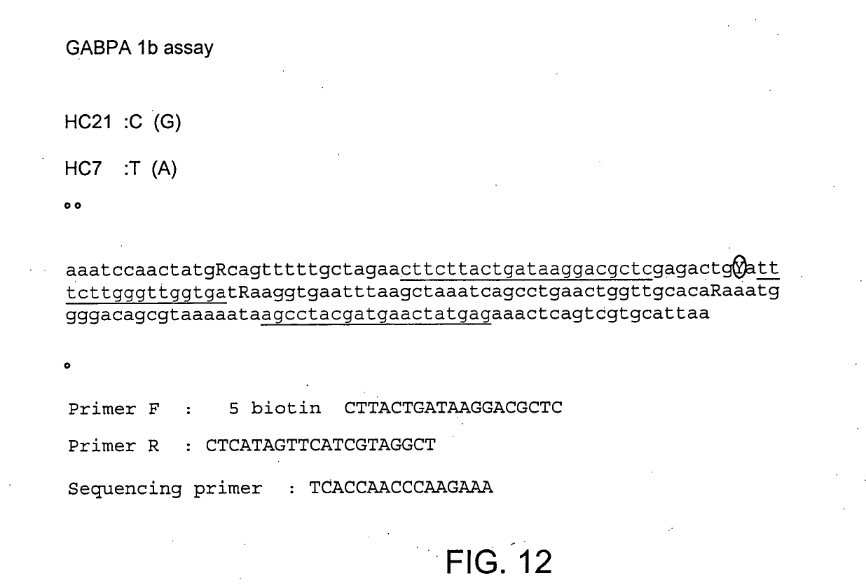

Using primer sequences GABPAF (5 biotin CTTACTGATAAGGACGCTC) and GABPAR (CTCATAGTTCATCGTAGGCT) (FIG. 12), the sample is subjected to PCR conditions. For example, providing 5.0 μl of amplification buffer, 200 μM dNTPs, 3 mM MgCl₂, 50 ng DNA, and 5 Units of Taq polymerase, 35 cycles of touchdown PCR (e.g., 94° C. for 30 seconds; 63-58° C. for 30 seconds; and 72° C. for 10 seconds) generates suitable amounts of amplification products for subsequent detection of sequence differences between the two paralogs. FIG. 12 demonstrates the optimized assay showing the primers used. FIGS. 3 and 7 show the positions (circled or indicated by arrow) used for quantification.

The amount of amplified products corresponding to the GABPA gene paralogue and GABPA was determined by assaying for single nucleotide differences which distinguish the two genes (see circled sequence in FIG. 12 or sequence marked by an arrow in FIG. 3). Preferably this is done by a pyrosequencing™ method, using sequencing primer GABPAS (TCACCAACCCAAGAAA).

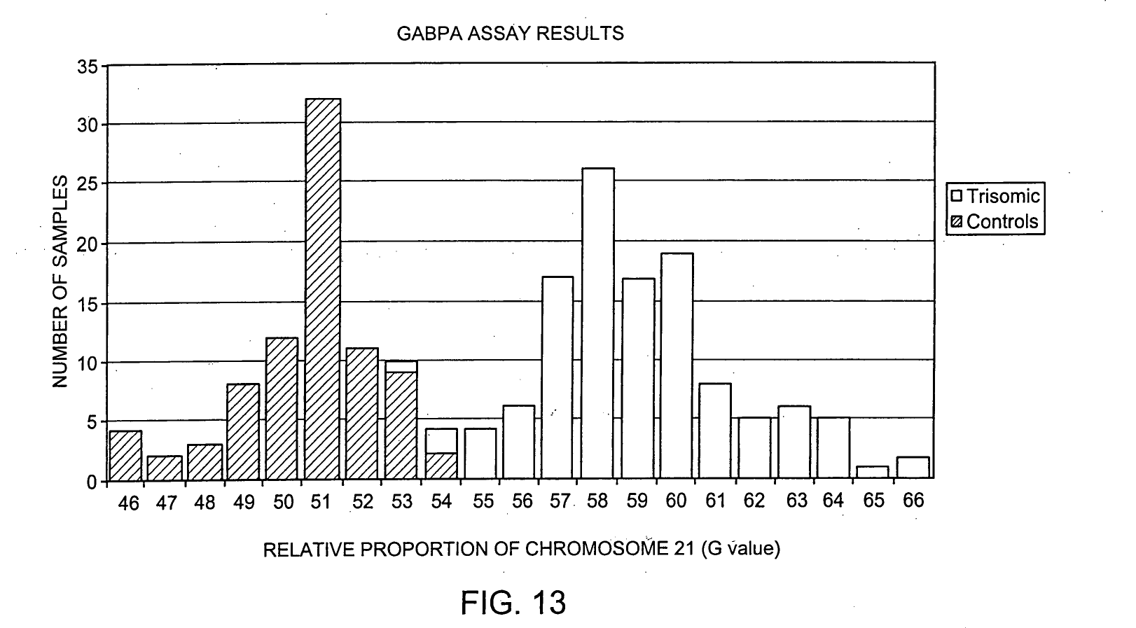

Samples were analyzed using a pyrosequencer. A threshold of 10 units per single nucleotide incorporation was set as a quality control for the DNA, below which the samples were discarded from the analysis. Following this procedure 169 samples were discarded and the remainder were analyzed. Although this threshold is quite conservative, assays with lower signal intensities produce less reliable quantifications. FIG. 13 shows the distribution of G values for the 230 samples analyzed. The G allele represents the relative proportion of chromosome 21. Control DNAs had an average G value of 51.11% with a Standard deviation of 1.3%. Trisomic individuals had an average value of 59.54% with a standard deviation of 1.90%. As seen from the graph the two groups are well separated. However for samples with values between 53.0-54.9 no clear diagnosis can be given. However, only 5% of samples fall within this interval and hence an unambiguous diagnosis can be given in 95% of the cases according to the data obtained.

In addition there were 4 samples for which a wrong diagnosis was given. Further analysis using microsatellite markers showed that 3 of these individuals had been misclassified, and hence were controls rather than trisomic individuals. The fourth sample (DS0006-F5) was confirmed to be trisomic and hence probably represents an error due to contamination in the reaction, since the same sample gave a correct result with the CCT8 assay.

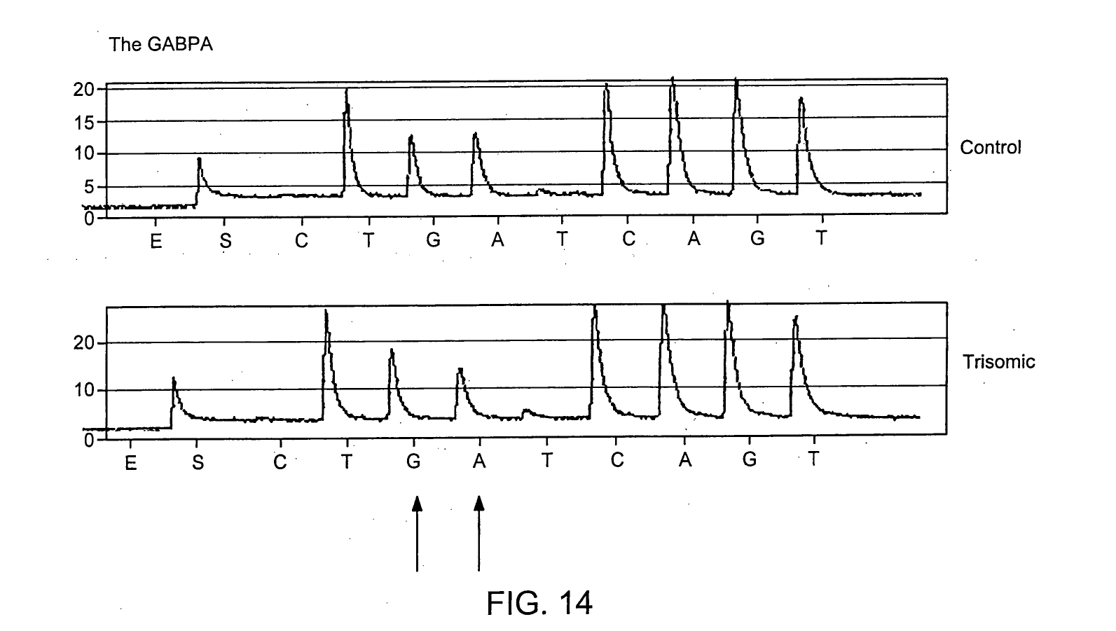

FIG. 14 shows typical programs for the GABPA assay. Arrows indicate positions used for chromosome quantification.

### Example 4

The following example describes a method for detecting Trisomy 21 according to the method of the invention, wherein one member of the paralogous gene pair is CCT8.

Trisomy 21 is detected by providing a sample comprising at least one cell from a patient (e.g., a fetus) and extracting DNA from the cell(s) using standard techniques.

DNA samples (trisomic and control samples) were incubated with a single pair of primers which will specifically anneal to both CCT8 (GenBank accession no. NM_{—}006585) and the CCT8 gene paralogue (GenBank accession no. LOC149003), paralogous genes located on chromosome 21 and chromosome 1, respectively, under standard annealing conditions used in PCR. Alignment of sequences of a CCT8 paralogue and CCT8 is shown in FIG. 4.

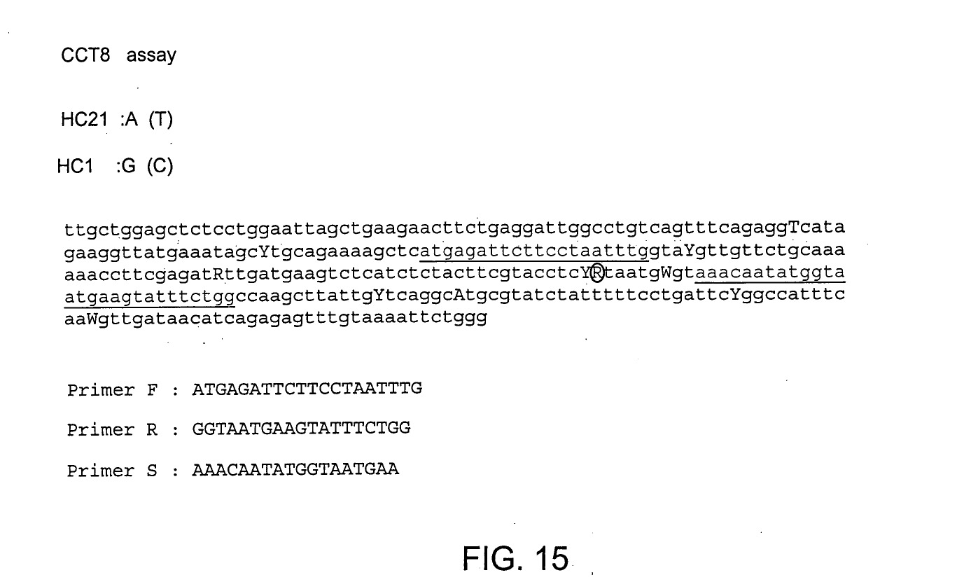

Using primer sequences CCT8F (ATGAGATTCTTCCTAATTTG) and CCT8R (GGTAATGAAGTATTTCTGG) (FIG. 15), the sample is subjected to PCR conditions. For example, providing 5.0 μl of amplification buffer, 200 μM dNTPs, 3 mM MgCl₂, 50 ng DNA, and 5 Units of Taq polymerase, 35 cycles of touchdown PCR (e.g., 94° C. for 30 seconds; 63-58° C. for 30 seconds; and 72° C. for 10 seconds) generates suitable amounts of amplification products for subsequent detection of sequence differences between the two paralogs. FIG. 15 demonstrates the optimized assay showing the primers used. FIGS. 4 and 15 demonstrate the position (circled or indicated by arrow) which was used for quantification.

The amount of amplified products corresponding to the CCT8 paralogue and CCT8 was determined by assaying for single nucleotide differences which distinguish the two genes (see circled sequence or sequence marked by arrow in FIG. 4 and 15). Preferably this is done by a pyrosequencing™ method, using sequencing primer CCT8S (AAACAATATGGTAATGAA).

Samples were analyzed using a pyrosequencer as described in example 3. Following this procedure 210 samples were discarded and the remainder were analyzed.

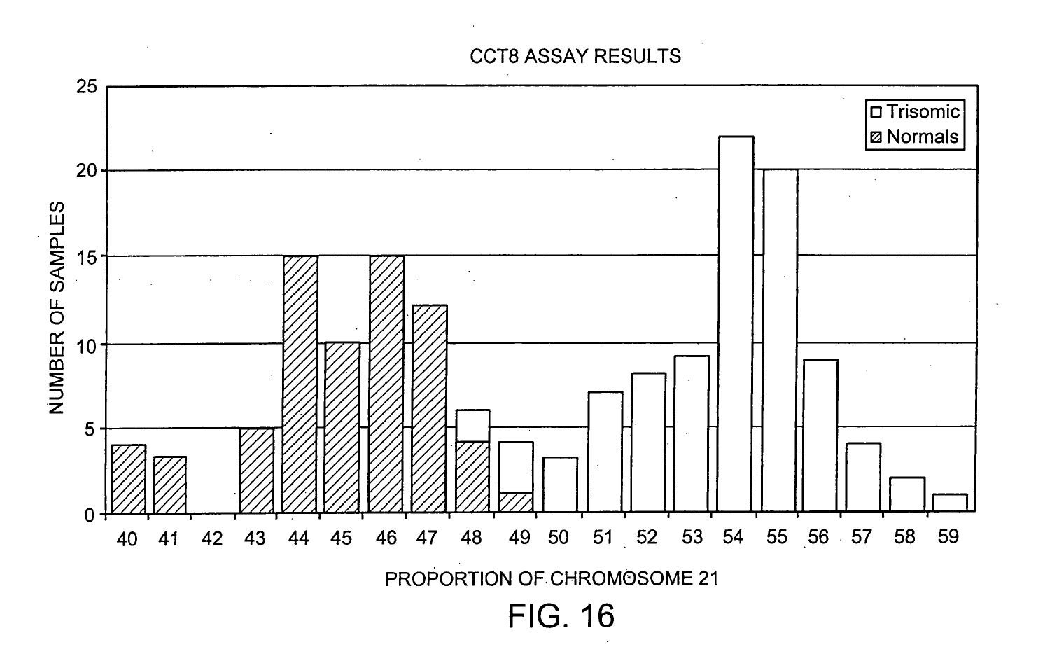

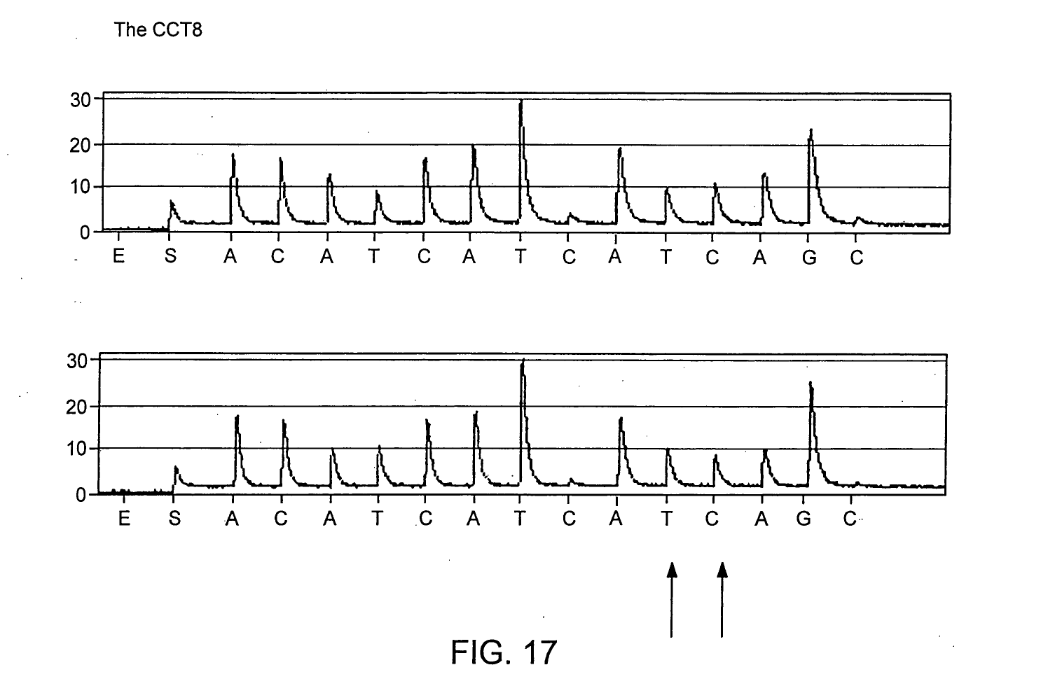

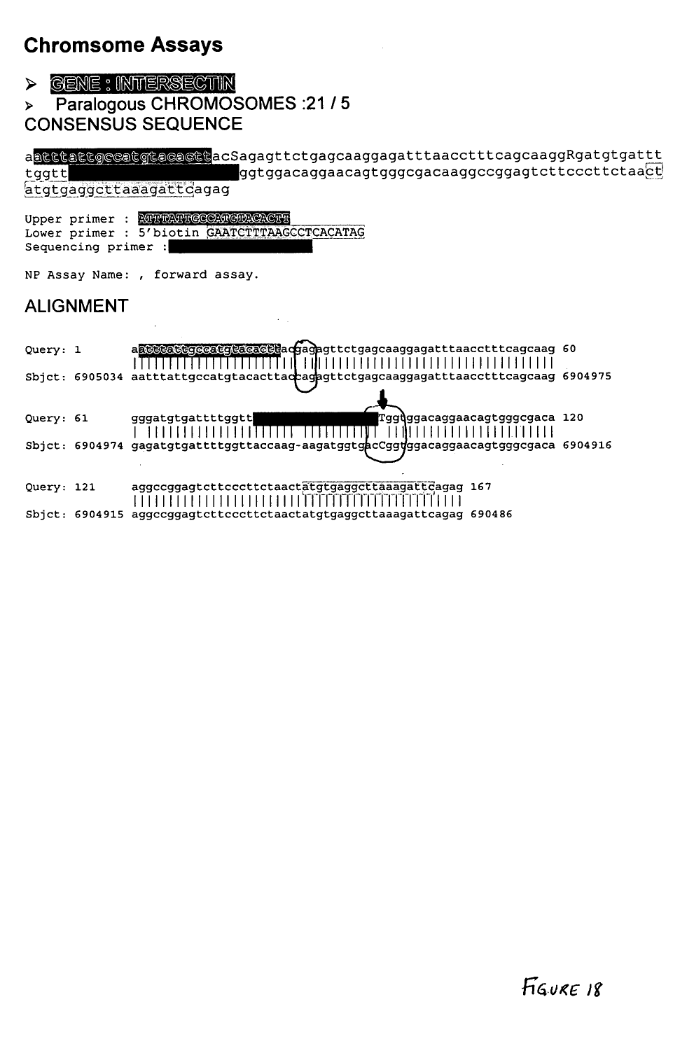

FIG. 16 shows the distribution of T values (proportion of HC21) for the 190 samples analyzed. The T allele represents the relative proportion of chromosome 21. As seen from the graph, the distribution is very similar to that of the GABPA assay, with well separated medians and a region in the middle for which no clear diagnosis can be made. In this case samples with values between 48-50 could not be diagnosed, but as in Example 3, only 5% of the samples fall within this range. In addition there were 2/190 samples for which a wrong diagnosis was given, probably as a result of contamination. FIG. 17 shows typical programs for the CCT8 assay. Arrows indicate positions used for chromosome quantification.

The data from the validation studies for the GABPA and CCT8 tests show that using each assay separately, 95% of the samples can be correctly diagnosed, with a 1-1.5% error rate of unknown origin (likely to be caused by contamination). However if both tests are considered together, the data show that 98% of the samples can be correctly diagnosed, (while for the remaining 2% no diagnosis can be given) and more importantly the 3 errors could be easily detected, as both assays gave contradictory results. This argues strongly for the use of the two tests in parallel to minimize the probability of a false diagnosis.

### Example 5

The following example describes a method of detecting aneuploidies by paralogous sequence quantification.

Samples

DNA samples from 50 trisomy 21 individuals that had been previously collected with informed consent in our laboratory were used for this study. Specific authorisation was requested to the ethics committee of the Geneva University Hospitals, for use of the DNA samples in this particular project. Fifteen fibroblast cell cultures from individuals with various chromosomal abnormalities were purchased from the Coriell Cell Repositories (GM03330, GM02948, GM00526, GM03538, GM02732, GM01359, GM00734, GM00143, GM03102, GM01250, GM09326, GM11337, GM00857, GM01176, GM10179). Sixty DNA samples of individuals carrying trisomies of chromosomes 13 and 18, and various sex chromosome abnormalities, were provided by Genzyme Corporation (Cambridge, Mass.). Finally, 50 normal individuals from the CEPH collection were used as additional controls.

Genomic DNA was prepared with either the PUREGENE whole blood kit (Gentra Systems Inc. Minneapolis, USA) or the QIAamp kit (Qiagen, Hilden, Germany).

Paralogous Sequence Quantification (PSQ)

PCR reactions with the selected primer pairs (Table 3) were set-up in a total volume of 25 μl containing 20 ng of genomic DNA, 5 pmol of each primer, and 200 μmol/L of dNTPs. 1.25 units of a standard Taq polymerase (Amersham Biosciences, Bukinghamshire, UK), or alternatively a ready made 2×PCR mastermix containing dUTP and N-uracil glycosylase (Eurogentec, Seraing, Belgium) with varying levels of MgCl₂ and DMSO depending on the assay (Table 3) were used.

TABLE 3
PCR primers and conditions.

| | | PCR, | | | |
|---|---|---|---|---|---|
| | | [Mg], | | | |
| Test | Gene ID | DMSO | F primer | R primer | S primer |
| Hsa 21a | ITSN | A; 3 | ATTTATTGCCATGTACACTT | bGAATCTTTAAGCCTCACATAG | ACCAAGAAAGATGGTGAC |
| Hsa 21b | GABPA | A; 3 | bCTTACTGATAAGGACGCTC | CTCATAGTTCATCGTAGGCT | TCACCAACCCAAGAAA |
| Hsa 13a | NUFIP1 | E; 1.5 | bGCTGAGCCGACTAGTGATT | AAGGGAAGCGAGGACGTAA | GGAAGCGAGGACGTA |
| Hsa 13b | STK24 | A; 1.5 | CGCTCTCGTCTGACATTT | bTCAGACATTTTTAGGTGG | CATTTGTTTGGAATCGT |
| Hsa 18a | KIAA1328 | A; 3, 5% | CGAAGGAAATGTCAGATCAA | bGACTCCATGGAGATTGAAG | TGTCAGATCAAGACACA |
| Hsa 18b | WBP11 | A; 3 | bGGAGGGACGGGAAGTAGAG | GTGAAGAAGCAGTGGATGTGCC | CAGAATCATCTTCATCAT |
| Hs XYa | ARSD | E; 3 | CGCCAGCAATGGATAC | bTGCAAAAGTGGTTTCGTTC | GGCCCTTCAGTGGA |
| Hs XYb | TGIF2LX | E; 3 | bAAGACAGCCCGGCGAAGA | ATTCCGGGAGAATGCGTCTGC | TGATAAACCAGTTAGAAATC |
| Hs XAa | TAF9L | E; 3 | bTGCCTAATGTTTTGTGATT | GACCCAAAACTACCTGTC | GTAAAACCCAACTG |
| Hs XAb | JM5 | E; 3 | CCCTGTGTGTCTCTAAACCAGC | bGGTGGCAGGGTCAGT | GAAACTGGTGGAGCTG |
| Gene ID refers to HUGO names for all the ‘query genes’. PCR refers to the PCR conditions used: A indicates that Amersham (Amersham Biosciences, Bukinghamshire, UK), and E Eurogentec (Eurogentec, Seraing, Belgium) PCR buffers and Taq polymerase were used. 3 or 1.5 indicates the final concentration of MgCl₂ and 5% indicates the final concentration (v/v) of DMSO. b at the start of the | | | | | |
| # primers indicates 5′biotinylated primer. F, R and S refer to forward, reverse and sequencing primers respectively. | | | | | |

PCR reactions were carried out on a T gradient thermocycler (Biometra, Göttingen, Germany), and cycling conditions consisted of a 2 min step at 50° C., and 10 min denaturation at 94° C. This was followed by 10 cycles of ‘touchdown PCR’ with a 20 s denaturation step at 94° C., a 20 s annealing step starting at 57° C. and decreasing by −0.5° C. per cycle, and an extension step at 72° C. for 20 s. The final 30 cycles were as before, but with a constant annealing temperature of 52° C., followed by a final elongation step of 72° C. for 5 min.

PCR products were purified, and annealed to an internal sequencing primer close to the PSM site to be quantified. The purification and pyrosequencing steps were performed following the instructions of the manufacturer (Pyrosequencing, Uppsala, Sweden).

Data Analysis.

The Pyrosequencing software directly outputs a quantitative value for the proportion of each PSM present in the PCR product. We used the percent of the ‘query’ chromosome as our statistic for all calculations. To determine the range of values that could be confidently diagnosed for every assay we calculated the 99% confidence for the distribution of control and affected individuals (bimodal distribution). Any sample with a value outside these limits was considered uncertain. Uncertain samples were treated either as false positive or as false negatives according to the known karyotypes, and this was used to estimate the sensitivity and specificity of each test using standard approaches (Fletcher et al., 1996, Clinical Epidemiology: The Essentials. Third ed. Baltimore, Williams and Wilkins).

In order to combine the two assays for each type of aneuploidy we normalised the distributions so that the average percent of the query chromosome for the control individuals was 50 (the expected outcome) for all of the assays. The mean of the two assays for each sample was then calculated.

To determine the reproducibility of our assays, we randomly selected a control and an affected sample for each autosomal aneuploidy, and a male and a female sample for the X vs. Y and X vs. A assays. 12 replicates were used for each sample for each assay: 4 on the same run with the same PCR mix, 4 on a second day with the same PCR mix as the first day, and 4 on a third day with a different batch of PCR mix and performed by a different operator. The coefficient of variation for same day, same PCR batch measurements (CV1), different day, same PCR batch measurements (CV2), and different day, different PCR batch measurements (CV3) were calculated.

Assay Design

To design paralogous sequence quantification (PSQ) assays, the first step entails the identification of paralogous sequences located on different chromosomes. One of the sequences must map to the chromosome of interest (or ‘query’ chromosome, for example chromosome 21), and the second to any other autosomal chromosome (‘reference’ chromosome).

To identify such paralogous sequences, all the known exons of chromosomes 13, 18, 21 and X, (http://www.ensembl.ore/), were batch blasted against the human genome. Matches with high scores (usually >350) and very low E values (<10⁻⁴⁰) where only two hits were observed: one to the ‘query chromosome’ and the second elsewhere in the genome (FIG. 28A) were selected.

The second step of the method involves the quantification of single nucleotide differences between the paralogous sequences (PSMs). For this we chose the Pyrosequencing method (Alderborn et al., 2000;10(8):1249-58) (www.pyrosequencing.com) that has been previously shown to be highly quantitative (Deutsch et al., 2003, Blood 102(2):529-34; Hochberg et al., 2003, Blood 101(1):363-9; Qiu et al., 2003, Biochem. Biophys. Res. Commun. 309(2):331-8; Neve et al., 2002, Biotechniques 32(5):1138-42.

To design pyrosequencing assays, the selected BLAST alignments for each of the ‘query chromosomes’ (FIG. 28B) were used to manually build a consensus sequence, which was entered into the Oligo 3 software to obtain a suitable pair of primers that match perfectly to both chromosomes (to minimise differences in the efficiencies of amplication), and span at least one PSM. Quantification of the PSM position by Pyrosequencing can be used to determine the relative dosage of the ‘query’ and ‘reference’ chromosomes (FIG. 28C).

For the detection of sex chromosome abnormalities we designed two types of assays: A. X vs. Y assays to quantify the ratio between the X and the Y chromosomes (using a paralogous sequence present in the X and Y chromosomes), B. X vs. Autosomal assays to obtain the ratio between the X and any autosomal chromosome. The theoretically expected values (Table 4) show that this strategy allows the identification of all common aneuploidies.

TABLE 4
Expected values. Expected theoretical values for all assays
expressed as percent of ‘query’ chromosome.

| Autosomal trisomies | | Sex chromosome abnormalities | | |
|---|---|---|---|---|
| | Expected | | Expected value | Expected value |
| Status | value(%) | Karyotype | X vs. A assay (%) | X vs. Y assay (%) |
| Control | 50 | 45 X0 | 33 | 100 |
| Trisomic | 60 | 46 XX | 50 | 100 |
| ▮ | ▮ | 46 XY | 33 | 50 |
| ▮ | ▮ | 47 XXY | 50 | 66 |
| ▮ | ▮ | 47 XYY | 33 | 33 |
| ▮ | ▮ | 47 XXX | 60 | 100 |

Assay Selection

4-5 assays per chromosomal abnormality that were pre-screened with a panel of 8 control and 8 aneuploid samples were originally designed. Each assay was tested using a number of PCR conditions (varying concentrations of MgCl₂, and DMSO, and two types of buffer as described in the methods section). From this analysis the assays for each chromosomal abnormality based on the following criteria were selected: a. The PSM quantification in control individuals should be close to 50%, indicating that both ‘alleles’ amplify with equal efficiency; b. There should be a clear, non-overlapping discrimination between control and aneuploid samples; c. There should be the least possible deviation from the mean.

Only a subset of the assays fulfilled these conditions, and most of the assays were sensitive to the PCR condition used (data not shown). Ultimately the two best assays for each chromosomal abnormality for further validation was selected.

Assay Results

The performance of 10 independent tests designed to detect trisomies of chromosomes 13, 18 and 21 as well as sex chromosome aneuploidies were selected. The means (percent of ‘query’ chromosome as our statistic) and standard deviations for all of the assays are shown in Table 5.

TABLE 5
Summary results for each assay. All statistics were
calculated using the percent of ‘query chromosome’ as
calculated by the PSQ software.

| Autosomal | | | | | | |
|---|---|---|---|---|---|---|
| Assays | Hsa 13a | Hsa 13b | Hsa 18a | Hsa 18b | Hsa 21a | Hsa 21b |
| Mean control | 49.6 | 43.4 | 51.3 | 48.7 | 51.9 | 41.8 |
| SD control | 1.6 | 1.7 | 1.8 | 1.6 | 1.4 | 1.2 |
| Mean trisomic | 58.7 | 52.5 | 60.6 | 55.9 | 60.2 | 51.4 |
| SD trisomic | 2.3 | 1.4 | 1.5 | 0.9 | 1.3 | 1.4 |
| number of | 93 | 91 | 90 | 92 | 107 | 110 |
| samples | | | | | | |
| # of uncertain | 6 | 7 | 7 | 6 | 8 | 5 |
| samples | | | | | | |
| Sensitivity | 0.86 | 0.92 | 1.00 | 0.93 | 0.92 | 0.96 |
| Speficity | 1.00 | 0.97 | 0.90 | 0.96 | 0.93 | 0.95 |

| Sex chromosome | | | | |
|---|---|---|---|---|
| Assays | Hs XYa | Hs XYb | Hs XAa | Hs XAb |
| Mean 46, XY | 50.5 | 53.9 | 31.1 | 36.1 |
| SD 46, XY | 1.7 | 1.0 | 1.8 | 1.3 |
| Mean value 46, XX | 91.3 | 97.4 | 44.0 | 48.8 |
| SD 46, XX | 2.4 | 0.7 | 2.0 | 1.7 |
| number of samples | 93 | 93 | 93 | 93 |

Typical results of normal and affected samples for each assay are shown in FIG. 29. In 8 out of the 10 assays the observed average values corresponded or were very close to the theoretically expected values (Tables 2 and 3), and for the two remaining assays (Hsa 13b and Hsa 21b) there was an approximate 10% downwards shift for both the control and affected group, that did not affect the performance of the tests. The sensitivity and specificity was similar across all the assays (Table 5), with no false positive or false negative calls, but with on average 7% of samples falling outside the set confidence thresholds, thus precluding a diagnosis.

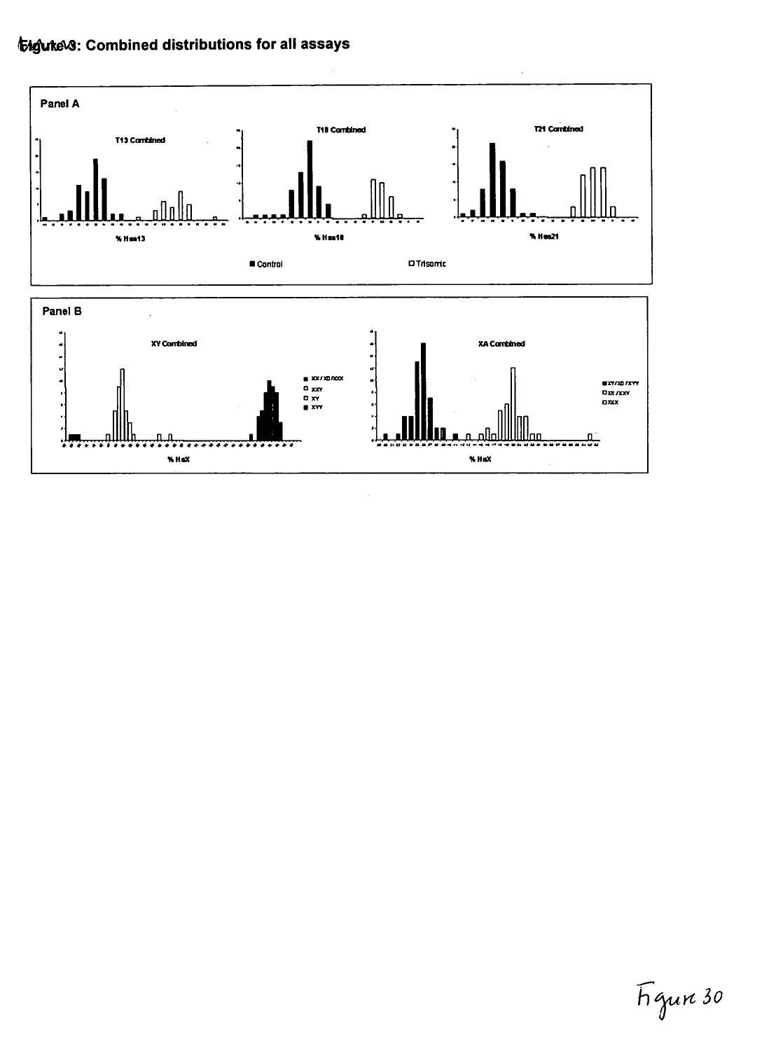

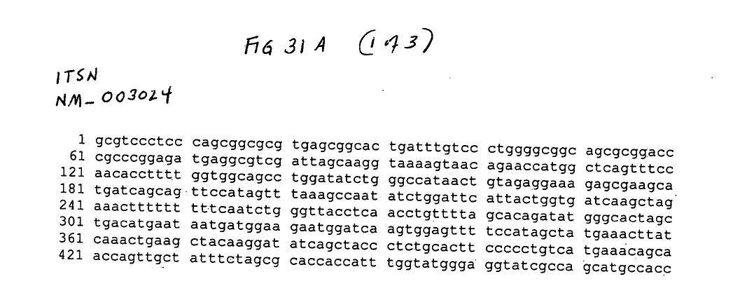

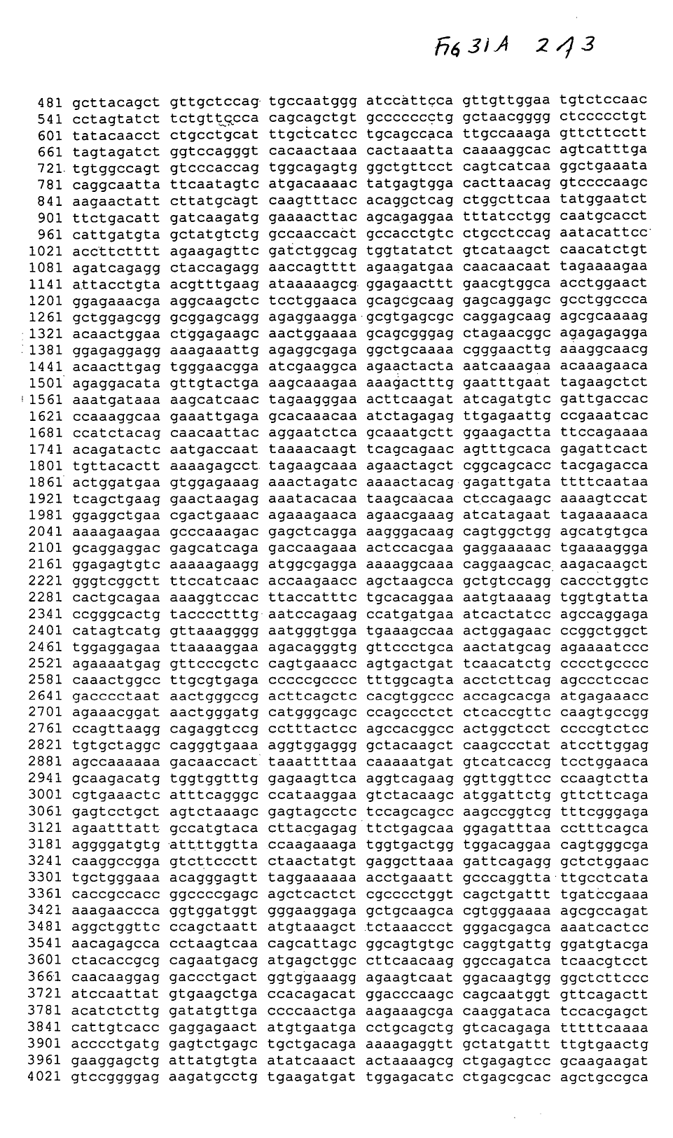

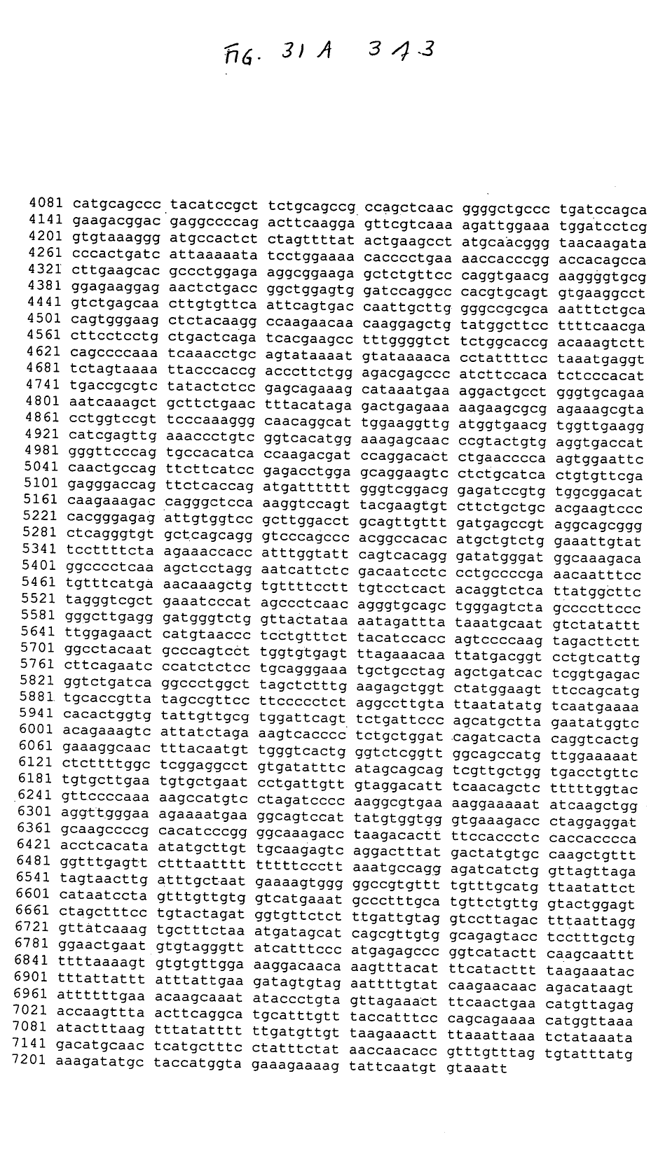

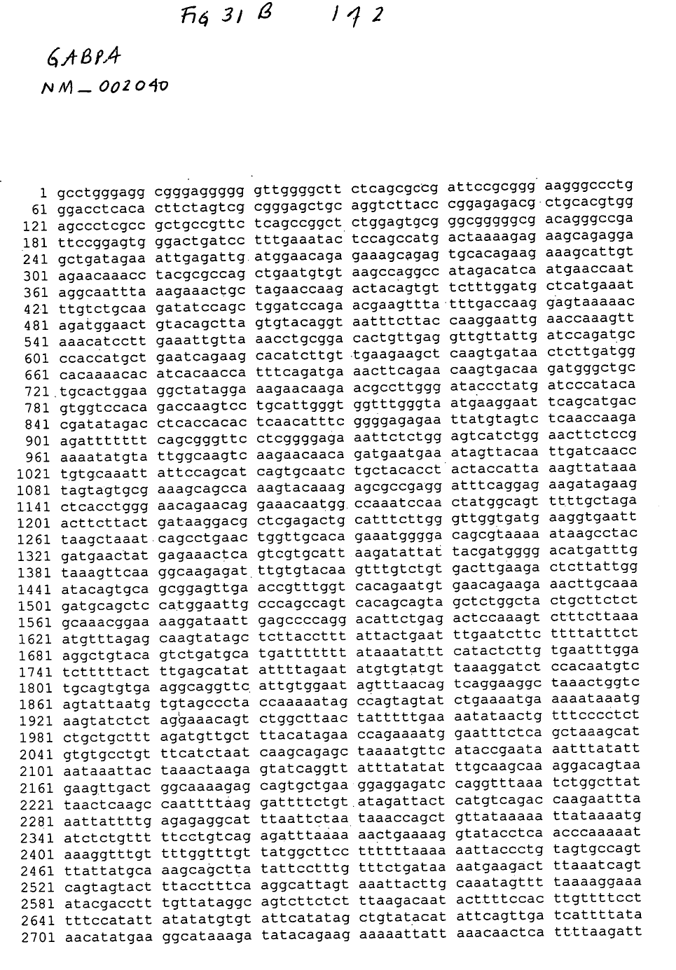

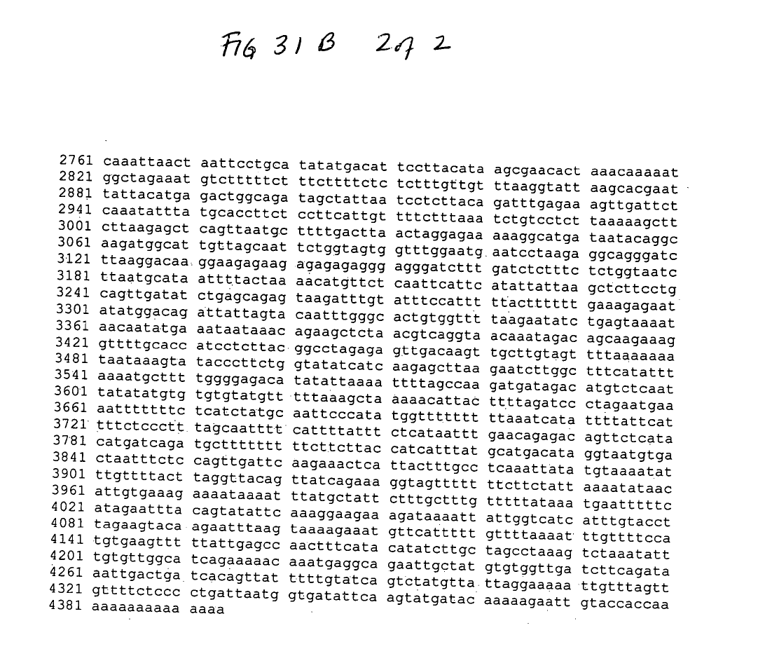

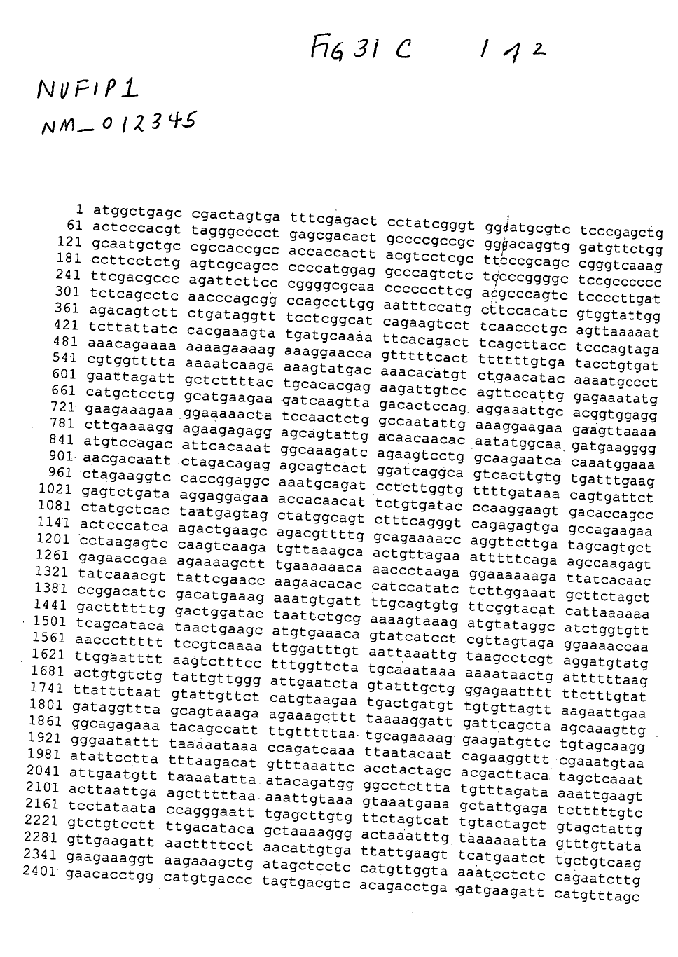

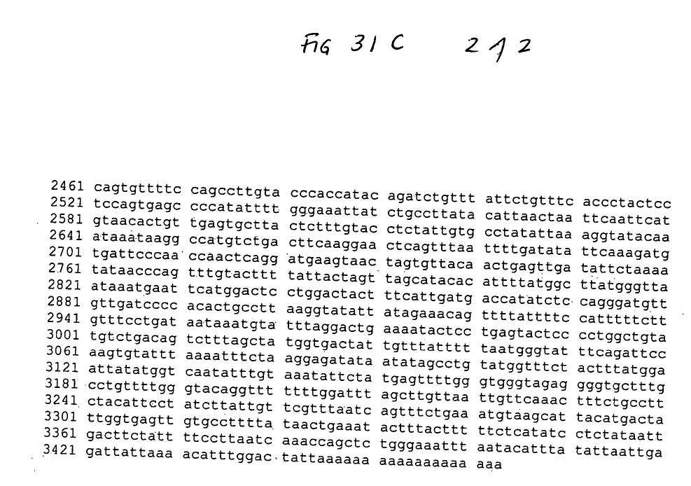

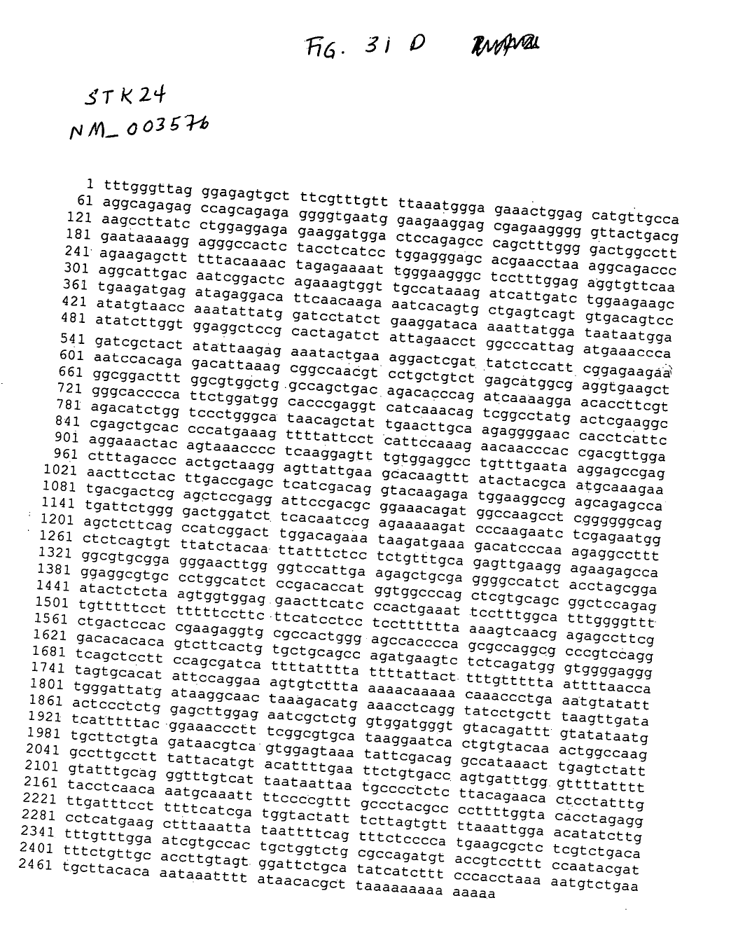

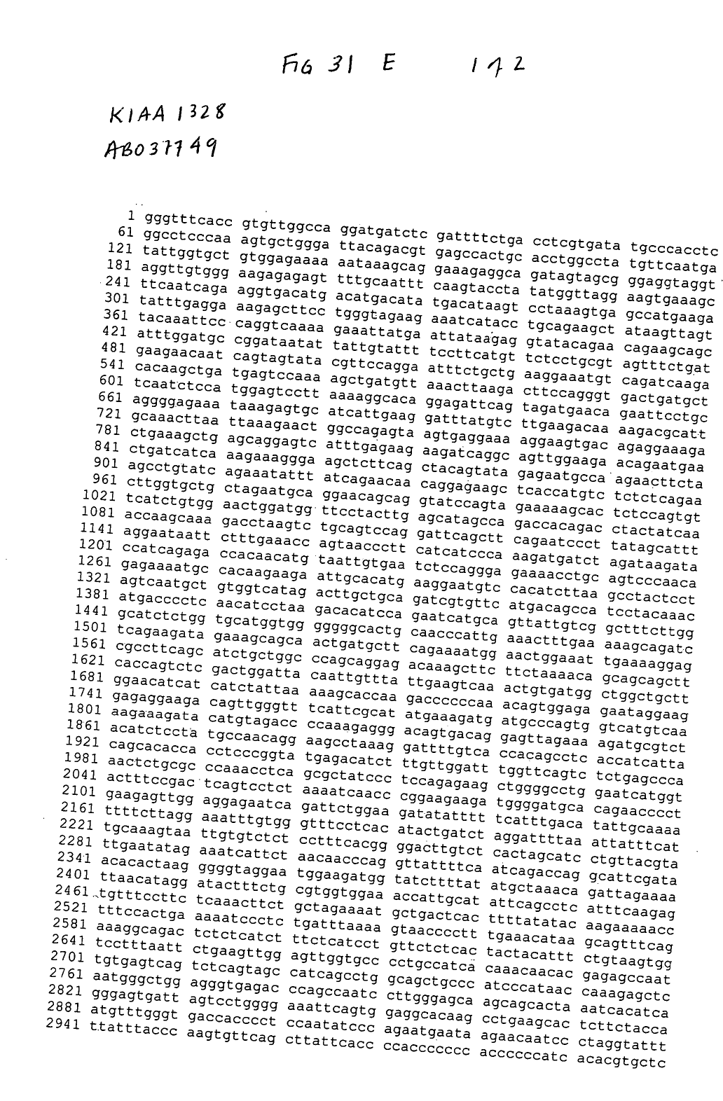

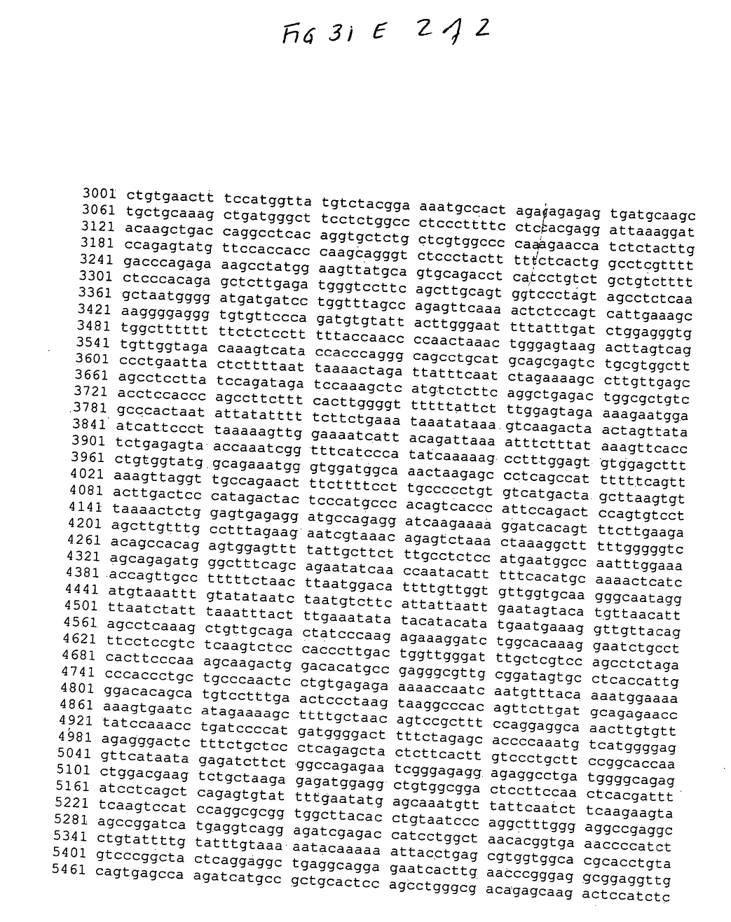

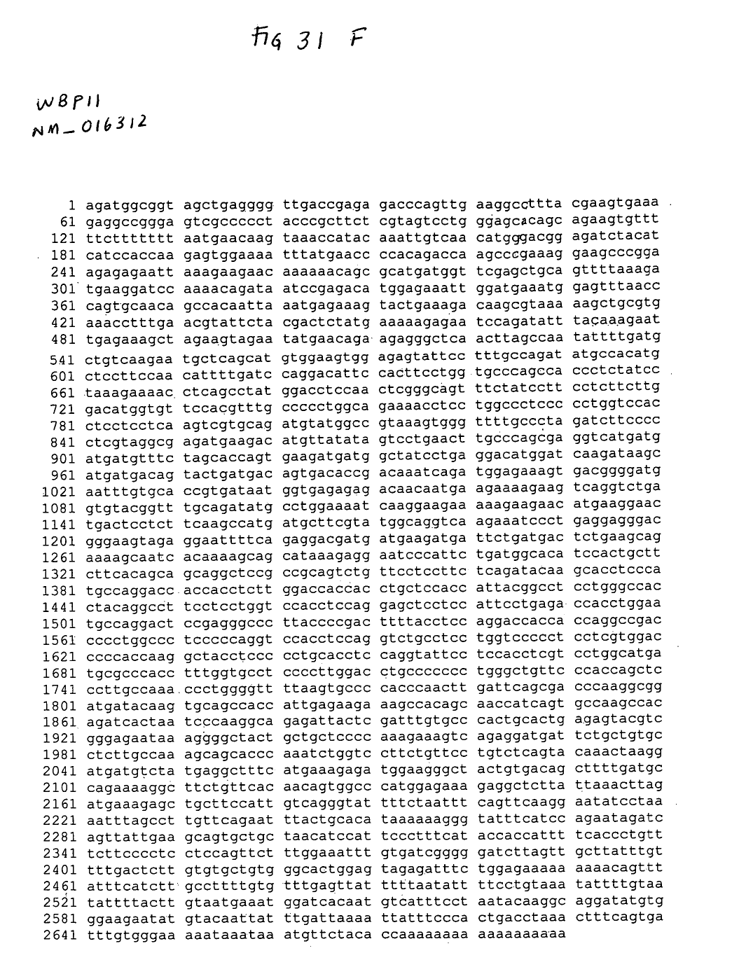

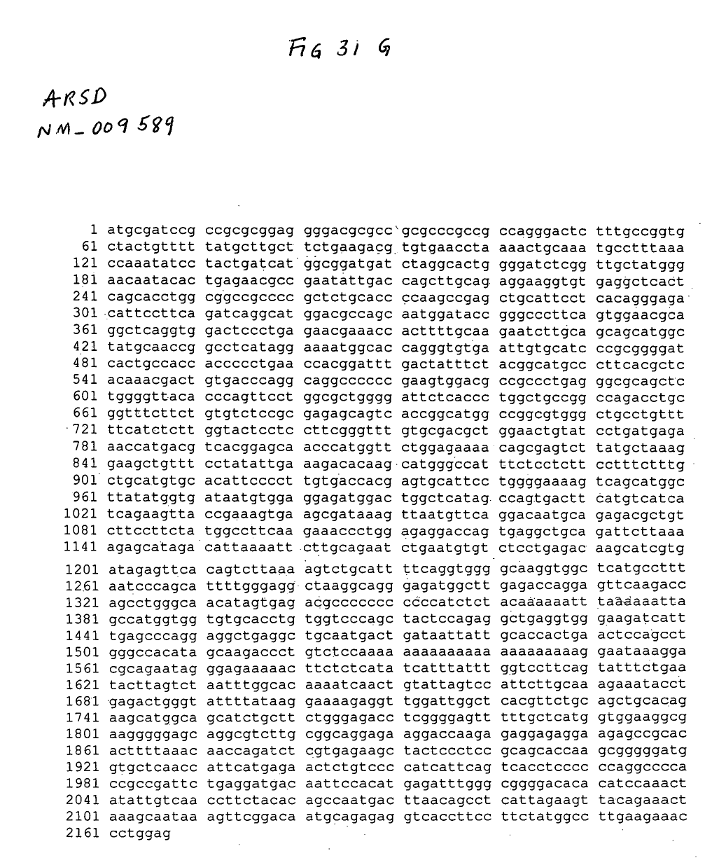

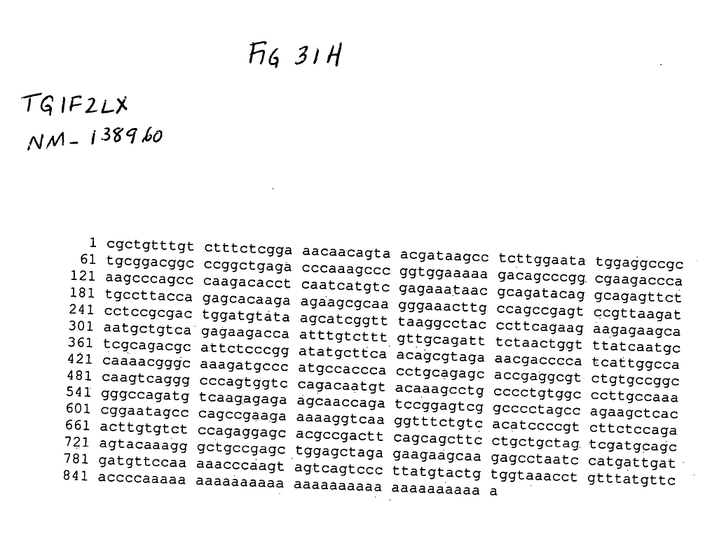

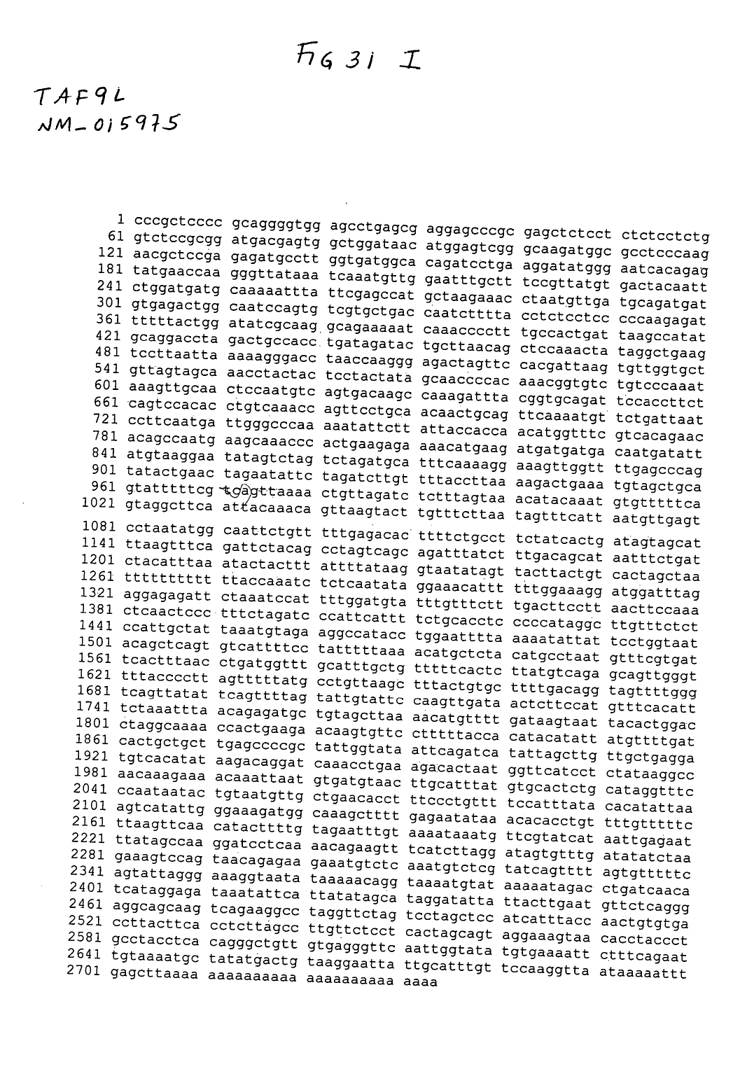

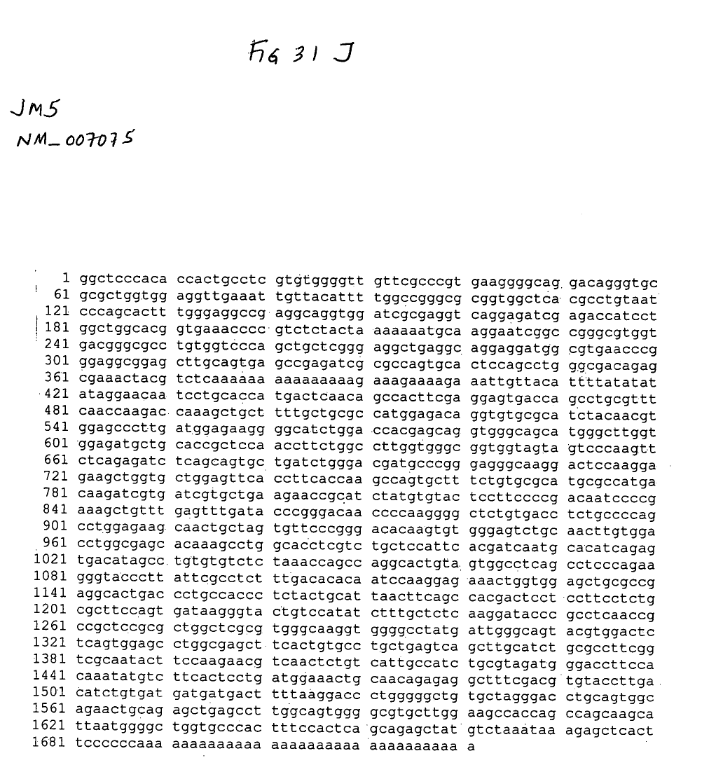

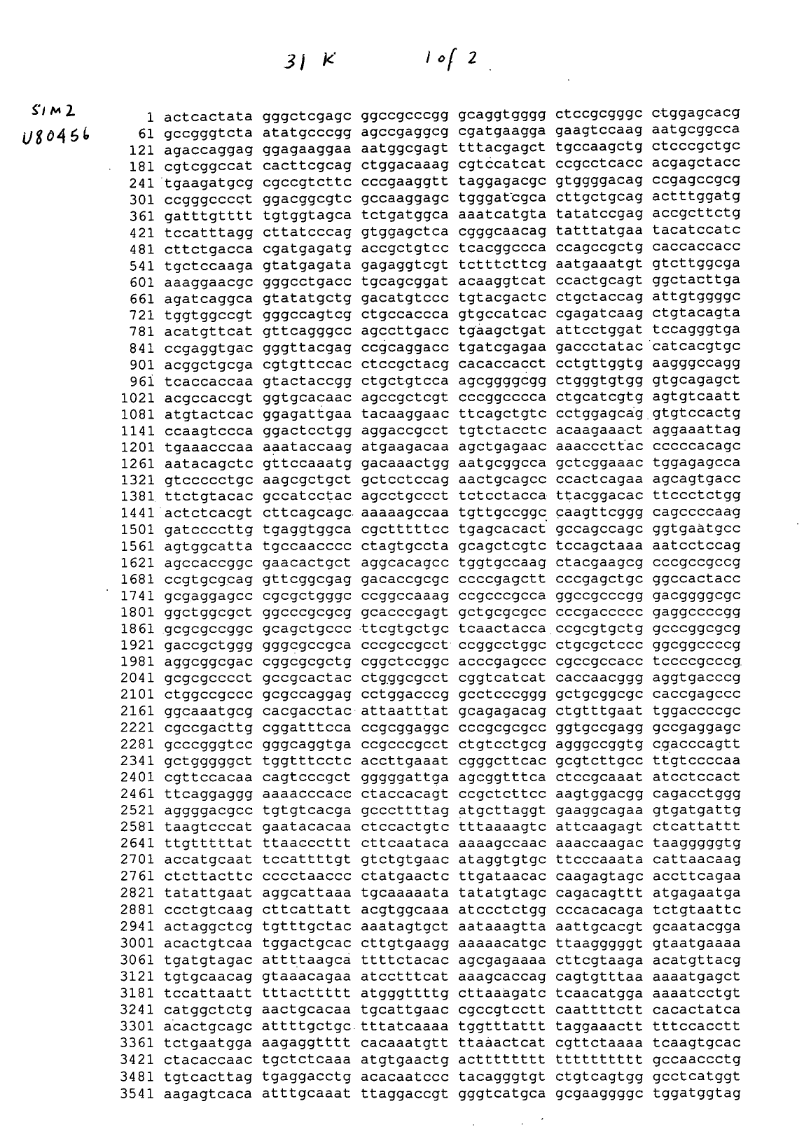

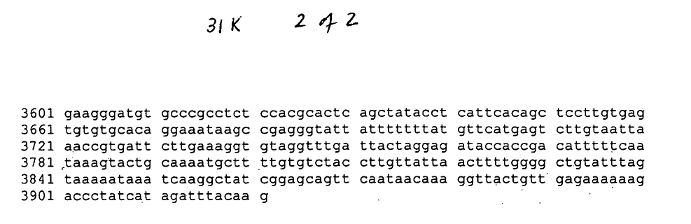

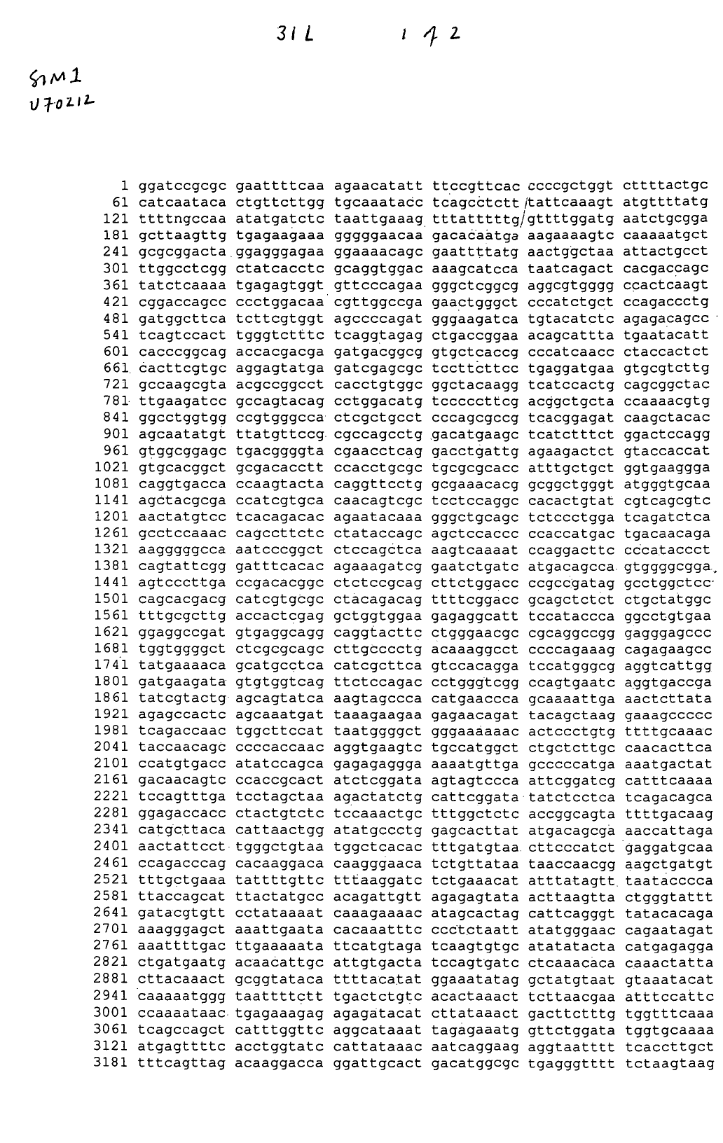

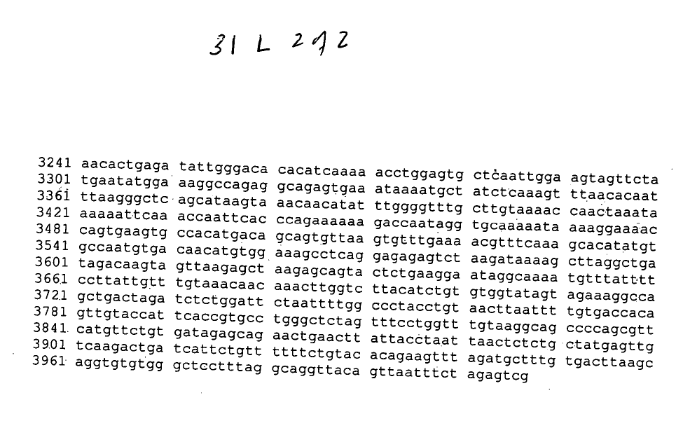

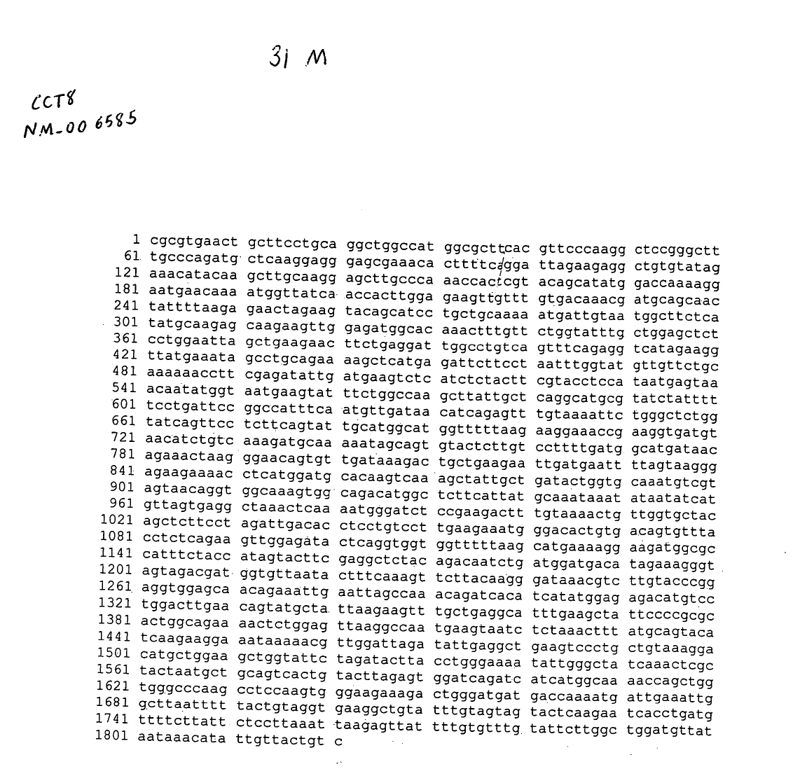

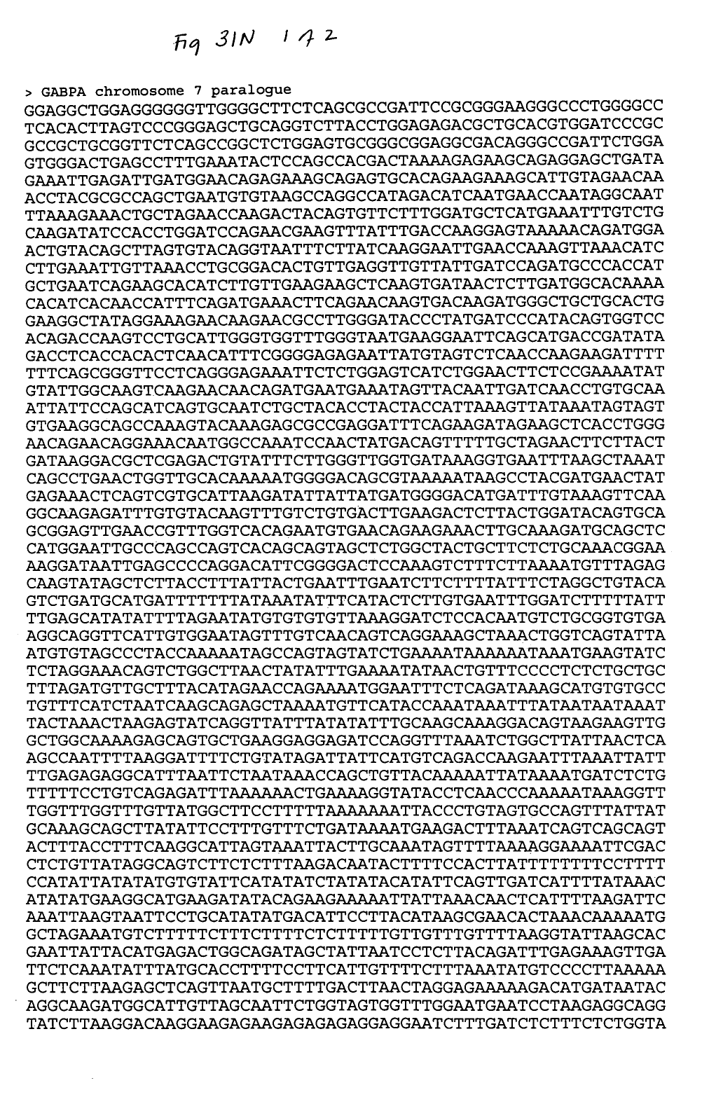

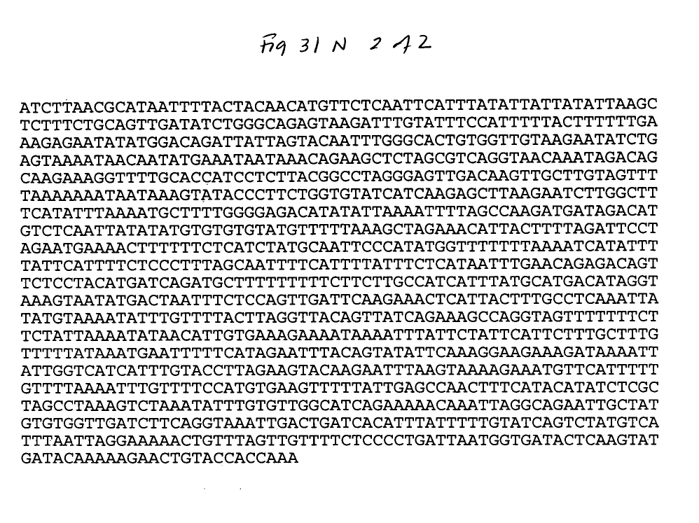

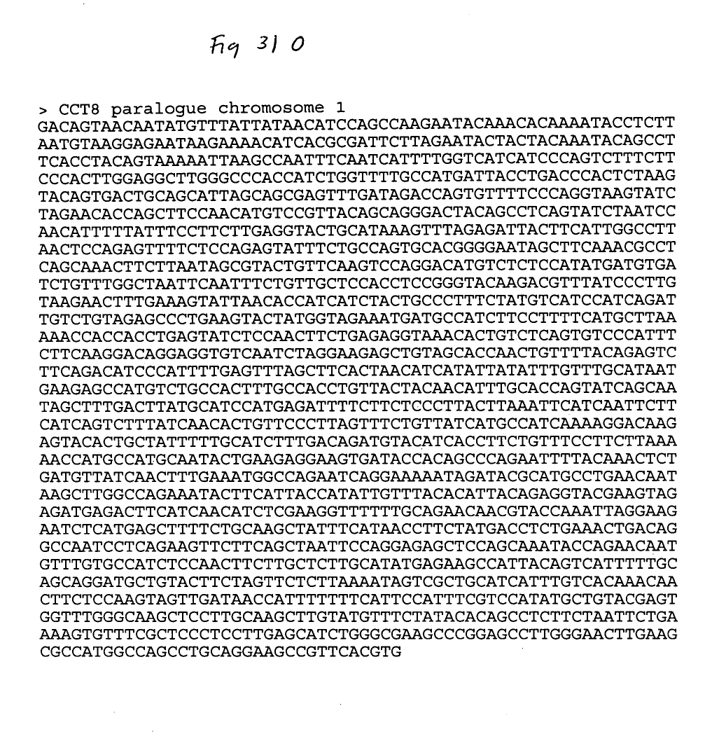

The results of the two independent assays for each aneuploidy, the results of both tests for each sample were integrated to generate a combined distribution. This resulted in a significant improvement in the separation between control and affected individuals, as seen by the greater sensitivities and specificities across all the tests (Table 6 and FIG. 30) and 99% of the samples being unambiguously diagnosed.

TABLE 6
Specificity and sensitivity of combined assays. Throughout the
study, 12 DNA samples repeatedly failed to amplify for at least
one of the assays, hence these samples were not further
considered.

| | Hsa 13 | Hsa 18 | Hsa 21 |
|---|---|---|---|
| Assay | combined | combined | combined |
| Mean control | 50 | 50 | 50 |
| SD control | 1.27 | 1.11 | 0.9 |
| Mean trisomic | 59.8 | 58.3 | 59.6 |
| SD trisomic | 1.32 | 1.11 | 1.05 |
| number of samples | 91 | 89 | 105 |
| # of uncertain samples | 1 | 0 | 0 |
| Sensitivity | 0.97 | 1 | 1 |
| Speficity | 1 | 1 | 1 |

Assays for Autosomal Aneuploidies

For trisomies of chromosomes 18 and 21, 89 and 105 samples respectively were tested, and used to obtain a correct and unambiguous diagnosis in all cases (Table 4). All 29 trisomy 13 samples and 47 trisomy 21 samples present were correctly identifed. Concerning the assays for trisomy 13, 91 samples were analysed, and out of these an unambiguous diagnosis was obtained for 90 samples. The status of one sample remained uncertain, since its combined value was outside the 99% confidence intervals. The two trisomy 13 assays for this sample were repeated, and again resulted in an ambiguous result, which could suggest that the individual is mosaic for trisomy 13. A 47,XX+13 karyotype was given for this sample, but since DNAs had been fully anonymised prior to the study, it was not possible to re-analyse the original karyotype.

Assays for Sex Chromosome Aneuploidies

93 samples for combined X vs. Y assays were analyzed and used to obtain a very clear separation between the 4 groups defined by the ratio between the X and Y chromosomes (FIG. 30B). In particular, the separation between the male group, and the group containing the females (46,XX; 45,X and 47,XXX that all have 100% of chromosome X) was very large, but this was expected and reflects the theoretical outcomes (Table 3). Nevertheless, since very few XXY and XYY individuals were present in the study, additional samples are required in order to establish the precise performance of these tests.

For the X vs. A combined assays, 91 samples were analyzed, out of which two samples 20 gave intermediate values that could not be diagnosed. However since these tests are partially redundant with the X vs. Y assays only one sample could not be fully resolved. One of the samples that had given a value of 41% in the X vs. A assay (hence an intermediate value between one and two X chromosomes), gave a value of 52% in the X vs. Y assay and thus was unambiguously diagnosed as a normal male. The second sample with an inconclusive diagnosis (X vs. A combined value of 43%) had given a value of 89% for the X vs. Y assay, and therefore it was not possible to discriminate between a 46,XX or a 45,XO diagnosis. The two X vs. A tests were therefore repeated and ued to obtain a combined value of 48% showing that individual is 46,XX.

Reproducibility

To estimate reproducibility of individual measurements, control and an affected sample for each aneuploidy (for the X vs. Y and X vs. A assays we picked individuals of different gender) were selected, and used to perform 12 replicate assays as detailed in the methods section. The results shown in table 7 demonstrate a high reproducibility for all of the assays, with a low coefficient of variation between same day and same batch replicates (0.7-4.3% of the mean), and for some assays a larger variation for inter batch replicates (up to 6.2%). These results indicate that some of the tests are sensitive to precise PCR conditions and thus to improve the reliability of the tests it might be advisable to work with frozen aliquots of a previously validated PCR mix containing the primers, buffer and dNTPs.

TABLE 7
Reproducibility of assays. Values indicate the coefficient of
variation for each of the assays. CV1 refers to same run, same PCR
batch mix variability, CV2 to different run, same PCR batch variability,
and CV3 to different run, different PCR batch variability.

| Assays | Control | | | Anueploid | | |
|---|---|---|---|---|---|---|
| Autosomal | CV1 | CV2 | CV3 | CV1 | CV2 | CV3 |
| Hsa 13a | 0.020 | 0.023 | 0.024 | 0.023 | 0.020 | 0.027 |
| Hsa 13b | 0.024 | 0.024 | 0.023 | 0.014 | 0.017 | 0.015 |
| Hsa 18a | 0.023 | 0.028 | 0.024 | 0.036 | 0.044 | 0.045 |
| Hsa 18b | 0.011 | 0.013 | 0.014 | 0.020 | 0.024 | 0.018 |
| Hsa 21a | 0.012 | 0.015 | 0.025 | 0.020 | 0.015 | 0.027 |
| Hsa 21b | 0.023 | 0.028 | 0.046 | 0.041 | 0.043 | 0.041 |
| Sex | Male | | | Female | | |
| chromosome | CV1 | CV2 | CV3 | CV1 | CV2 | CV3 |
| Hs XYa | 0.042 | 0.030 | 0.062 | 0.007 | 0.007 | 0.009 |
| Hs XYb | 0.022 | 0.017 | 0.029 | 0.016 | 0.012 | 0.034 |
| Hs XAa | 0.033 | 0.032 | 0.034 | 0.036 | 0.044 | 0.039 |
| Hs XAb | 0.038 | 0.044 | 0.058 | 0.021 | 0.040 | 0.060 |

In this study we present the paralogous sequence quantification approach, PSQ, as an alternative method for rapid and efficient detection of targeted aneuploidies that does not rely on the use of polymorphic markers. Ten different assays, designed for the identification of autosomal trisomies of chromosomes 13, 18 and 21 and sex chromosome number abnormalities were tested. We performed a retrospective study on 175 DNAs that were selected to include a relatively large number of aneuploid samples, in order to evaluate the sensitivity and specificity of the tests.

The performance of individual assays was characterised by no false negative or false positive, but a certain number of samples (7% on average) fell outside the 99% confidence intervals, for which an unambiguous diagnosis could not be established.

When combining the two tests for each chromosomal disorder, there was a significant improvement in the separation between control and affected samples, resulting in increased sensitivities and specificities across all tests, and the correct identification of 118 out of 120 abnormal samples present in the study. The remaining two samples were inconclusive after the first run and were subsequently re-tested, allowing an unambiguous diagnosis for one of the two, whereas the second sample remained uncertain, and could possibly originate from an individual with mosaicism.

Eight out of the 10 assays gave average values that were very close to the theoretically expected value. This shows that the strategy of using co-amplification of paralogous sequences with a single pair of primers that match perfectly at both loci, resulted in almost identical amplification efficiencies, and importantly, that end-point measurements using the Pyrosequencing method is a quantitative and reliable technique, consistent with previously published results Deutsch et al. 2003, supra; Hochberg et al., 2003, supra; Qiu et al., 2003, supra; Neve et al., 2002, supra. Selected samples for each assay were measured 12 times in order to evaluate the reproducibility of the tests. The intra and inter run variation between measurements was low, when the PCR mixes were from the same batch. Inter-batch variances were higher for some assays, suggesting that even small differences in the PCR mix resulting from inaccurate pipeting can have an effect. Our results suggests that in order to optimise the reliability of the procedure it might be necessary to make batches of PCR mix that can be tested and stored prior to use.

The first generation design of this test requires 10 separate PCR reactions per sample, which significantly reduces the sample throughput and increases the probability of handling errors. However, since the Pyrosequencing technology allows for a certain degree of multiplexing, the subsequent improvements of these assays should consist of no more that 3 or 4 PCR reactions per sample. Even with the current protocol, a single operator can handle at least 30-40 samples a day, and report results in less than 48 hours, which should cover the needs of most diagnostic laboratories.

Alternative molecular methods for the diagnosis of aneuploidies have been recently developed (Hulten et al., 2003, Reproduction, 126(3):279-97; Armour et al., 2002, Human Mutation 20(5):325-37). PCR based methods such as QF-PCR (Verma et al., 1998, Lancet 352(9121):9-12; Pertl et al., 1994, Lancet 343(8907):1197-8; Mann et al., 2001, Lancet 358(9287):1057-61; Adinolfi et al., 1997, Prenatal Diagnosis 17(13):1299-311), multiple amplifiable probe hybridization (MAPH) (Armour et al., 2000, Nucleic Acids Res 28(2):605-9), multiplex probe ligation assay (MPLA) (Slater et al., 2003, J Med Genet 40(12)907-12; Schouten et al., 2002 30(12:e57) and PSQ (presented herein) all have the advantage of being inexpensive, efficient in terms of labour and high-throughput. QF-PCR which is based on the use of polymorphic markers, is by far the most established of all the PCR based techniques, however it has a number of shortcomings, since some individuals can be homozygous at all sites, and the informativeness of markers can vary across different populations. Despite these problems, QF-PCR has been successfully implemented in several diagnostic laboratories (Mann et al., 2001, supra; Pertl et al., 1999, J Med Genet 36(4):300-3) and protocols using single nucleotide polymorphisms (SNPs) are currently being developed. MAPH and MPLA (both based on size specific probe design, co-amplification and size separation by capillary electrophoresis) do not make use of polymorphic markers and in principle work on all individuals. These two approaches have the advantage of allowing the simultaneous analysis of up to 40 loci using size specific probes that can be efficiently resolved by capillary electrophoresis, but initial results have shown up to 8 probes per chromosome are needed to obtain reliable results Slater et al. 2003, supra).

The major drawback of all PCR based tests is that they are targeted to specific regions of the genome, hence rare chromosomal abnormalities and balanced translocations will be missed. In addition low-level mosaicism, which can have significant clinical consequences, is difficult to detect with any DNA (rather than cell) based method.

Non PCR-based technologies such as comparative genome hybridization (CGH) have recently shown encouraging results (Veltman et al., 2002, Am J Hum Genet 70(5):1269-76; Snijders et al., 2001 Nat Genet 29(3):263-4) and the development of high-resolution arrays will surely become a powerful tool for the molecular diagnosis of DNA copy number abnormalities. However current protocols are considerably labour intensive and costly, hence its application as a routine diagnostic technique is not yet feasible.

The important debate of whether molecular tests should be used as ‘stand-alone’ tests (thus replacing karyotyping altogether) is a complex issue and has been discussed at length elsewhere (Hulten et al., 2003, Reproduction 126(3):279-97). A consensus however seems to be forming that molecular tests might be appropriate as stand-alone, for the low-risk group of women that are tested only on the basis of maternal age (this group constitutes the large majority of cases) and for which trisomies of chromosome 13, 18 and 21 and XY aneuploidies account for up to 99.9% of the disease-associated abnormalities.

No one single molecular method seems to be obviously superior to the rest, since all have advantages and disadvantages. Our data suggest that PSQ is a robust, easy to interpret and easy to set-up method for the diagnosis of common aneuploidies, that should represent a very competitive alternative for widespread use in routine diagnostic laboratories.

The practice of the present invention will employ, unless otherwise indicated, conventional techniques of molecular biology, cell biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); (Harlow, E. and Lane, D.) Using Antibodies: A Laboratory Manual (1999) Cold Spring Harbor Laboratory Press; and a series, Methods in Enzymology (Academic Press, Inc.); Short Protocols In Molecular Biology, (Ausubel et al., ed., 1995).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method for detecting risk of a chromosomal imbalance, comprising:
providing a sample of nucleic acids from an individual;
amplifying a first sequence at a first chromosomal location to produce a first amplification product;
amplifying a second sequence at a second chromosomal location to produce a second amplification product, said first and second amplification products comprising greater than about 80% identity, and comprising at least one nucleotide difference at a least one nucleotide position;
determining the ratio of said first and second amplification products; wherein a ratio which is not 1:1 is indicative of a risk of a chromosomal imbalance.

2. The method according to claim 1, wherein said amplifying is performed using PCR.

3. The method according to claim 1, wherein said first and second sequence are amplified using a single pair of primers.

4. The method according to claim 1, wherein said first and second chromosomal location are on different chromosomes.

5. The method according to claim 1, wherein said first and second sequences are paralogous sequences.

6. The method according to claim 1, wherein said first and second amplification products are the same number of nucleotides in length.

7. The method according to claim 1, further comprising identifying a first nucleotide at said at least one nucleotide position in said first amplification product and identifying a second nucleotide at said at least one nucleotide position in said second amplification product.

8. The method according to claim 7, wherein said identifying is performed by sequencing said first and second amplification product.

9. The method according to claim 8, wherein said sequencing is pyrosequencing™.

10. The method according to any one of claims 7-9, further comprising determining the amount of said first and second nucleotide at said at least one nucleotide position in said sample, wherein the ratio of said first and second nucleotide is proportional to the dose of said first and second sequence in said sample.

11. The method according to claim 10, further comprising the step of determining the amount of a nucleotide at a nucleotide position in said first and second amplification product comprising an identical nucleotide.

12. The method according to claim 1, wherein said chromosome imbalance is a trisomy.

13. The method according to claim 12, wherein said trisomy is trisomy 21.

14. The method according to claim 1, wherein said chromosome imbalance is a monosomy.

15. The method according to claim 1, wherein said chromosome imbalance is a duplication.

16. The method according to claim 1, wherein said chromosome imbalance is a deletion.

17. The method according to claim 3, wherein said primers are coupled with a first member of a binding pair for binding to a solid support on which a second member of a binding pair is bound, said second member capable of specifically binding to said first member.

18. The method according to claim 17, further comprising providing said solid support comprising said second member and binding said primers comprising said first member to said support.

19. The method according to claim 17, wherein said binding is performed prior to said amplifying.

20. The method according to claim 18, wherein said binding is performed after said amplifying.

21. The method according to claim 1, wherein said first sequence comprises the sequence of SIM1 and said second sequence comprises the sequence of SIM2.

22. The method according to claim 1, wherein said sample comprises at least one fetal cell.

23. The method according to claim 1, wherein said sample comprises somatic cells.

24. The method according to claim 1, wherein said first sequence comprises the sequence of a CCT8 paralogue and the second sequence comprises the sequence of CCT8.

25. The method according to claim 1, wherein said second sequence comprises the sequence of C210RF19.

26. The method according to claim 1, wherein said second sequence comprises the sequence of DSCR3.

27. The method according to claim 1, wherein said second sequence comprises the sequence of KIAA0958.

28. The method according to claim 1, wherein said second sequence comprises the sequence of TTC3.

29. The method according to claim 1, wherein said second sequence comprises the sequence of ITSN1.

30. The method according to claim 1, wherein said first sequence comprises the sequence of a RAP2A paralogue and the second sequence comprises the sequence of RAP2A.

31. The method according to claim 1, wherein said first sequence comprises the sequence of a CDK8 paralogue and the second sequence comprises the sequence of CDK8.

32. The method according to claim 1, wherein said first sequence comprises the sequence of an ACAA2 paralogue and the second sequence comprises the sequence of ACAA2.

33. The method according to claim 1, wherein said first sequence comprises the sequence of an ME2 paralogue and the second sequence comprises the sequence of ME2.

34. The method according to claim 1 wherein said first sequence comprises the sequence of an intersectin paralogue and the second sequence comprises the sequence of intersectin.

35. The method of claim 34, wherein said intersectin paralogue comprises the sequence presented in FIG. 18.

36. The method according to claim 3, wherein said pair of primers comprises ITSNF (ATTATTGCCATGTACACTT, SEQ ID NO 7) and ITSNR (GAATCTTTAAGCCTCACATAG, SEQ ID NO 8).

37. The method according to claim 1 wherein said first sequence comprises the sequence of a GABPA paralogue and the second sequence comprises the sequence of GABPA.

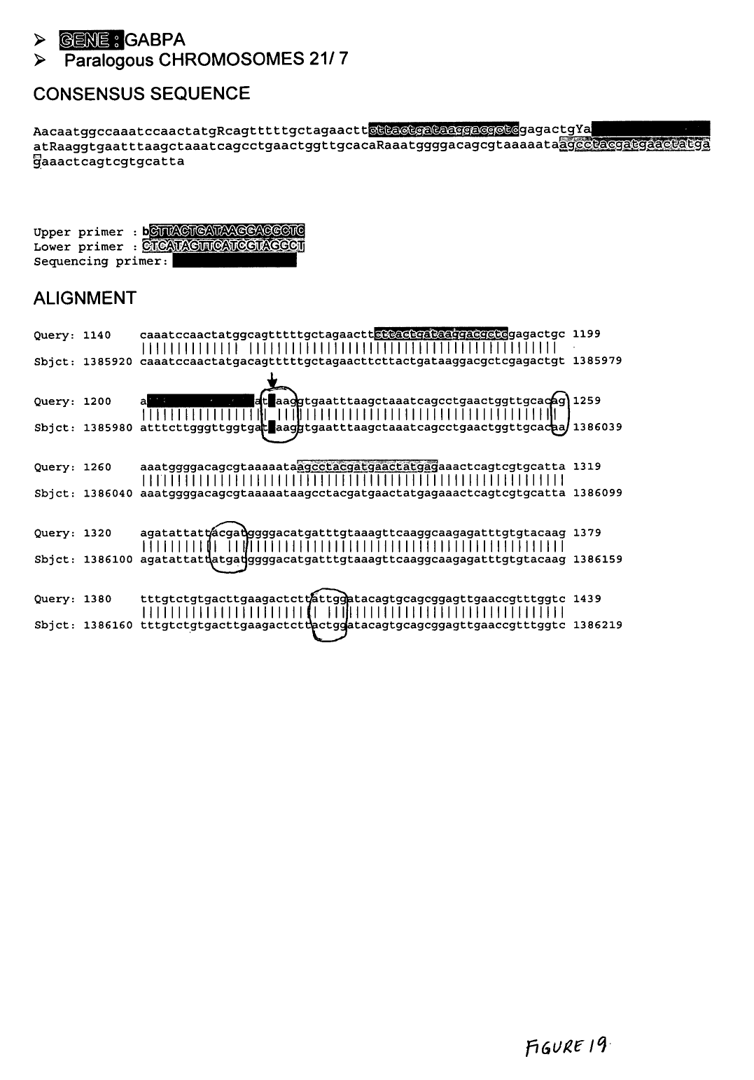

38. The method of claim 37, wherein said GABPA paralogue comprises the sequence presented in FIG. 19.

39. The method according to claim 3, wherein said pair of primers comprises GABPAF (CTTACTGATAAGGACGCTC, SEQ ID NO 3) and GABPAR (CTCATAGTTCATCGTAGGCT, SEQ ID NO 4).

40. The method according to claim 1 wherein said first sequence comprises the sequence of a NUFIP1 paralogue and the second sequence comprises the sequence of NUFIP1.

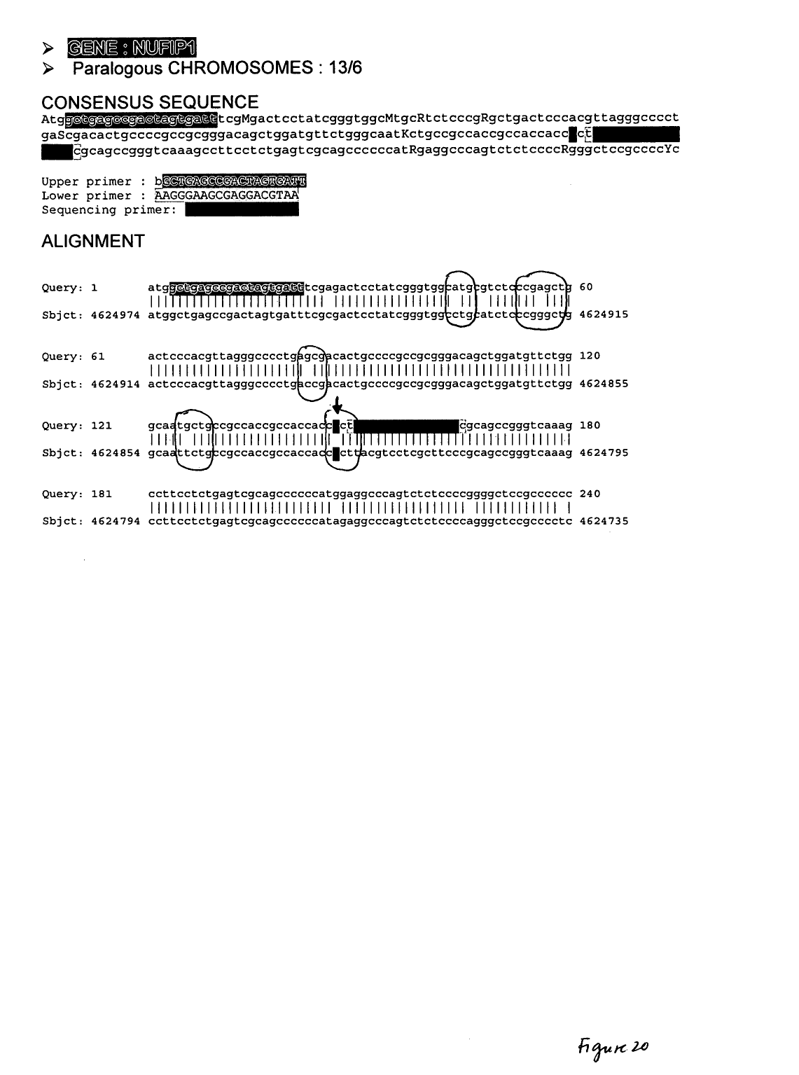

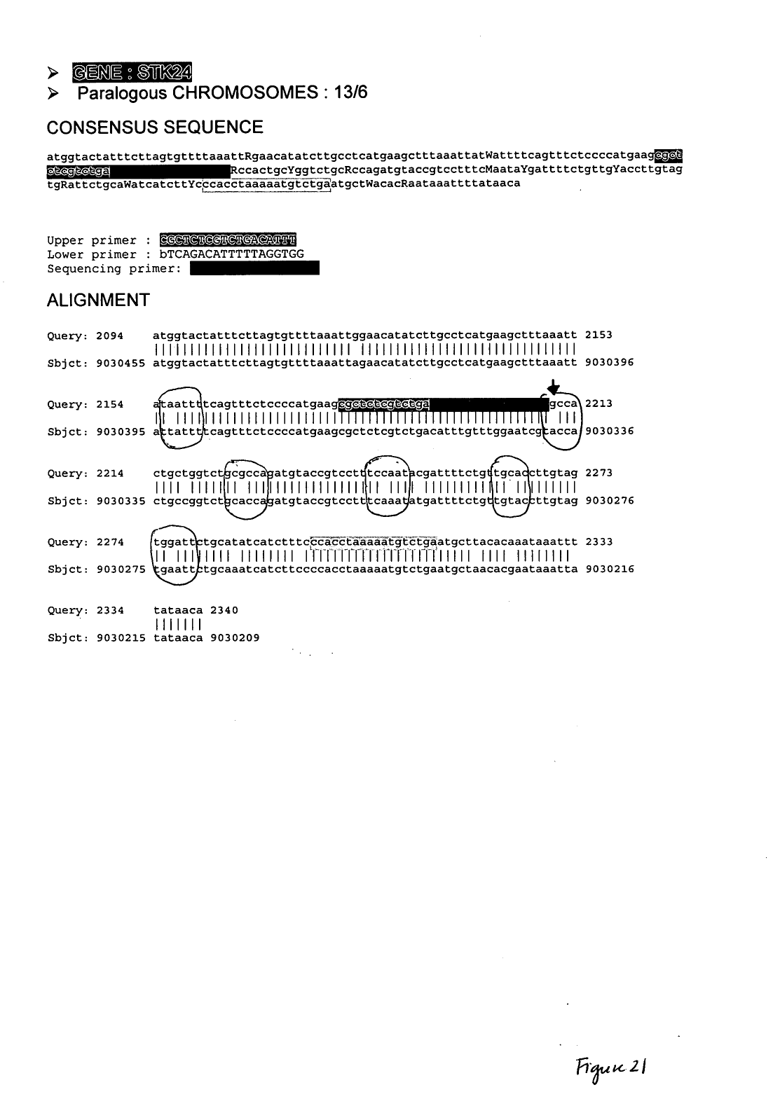

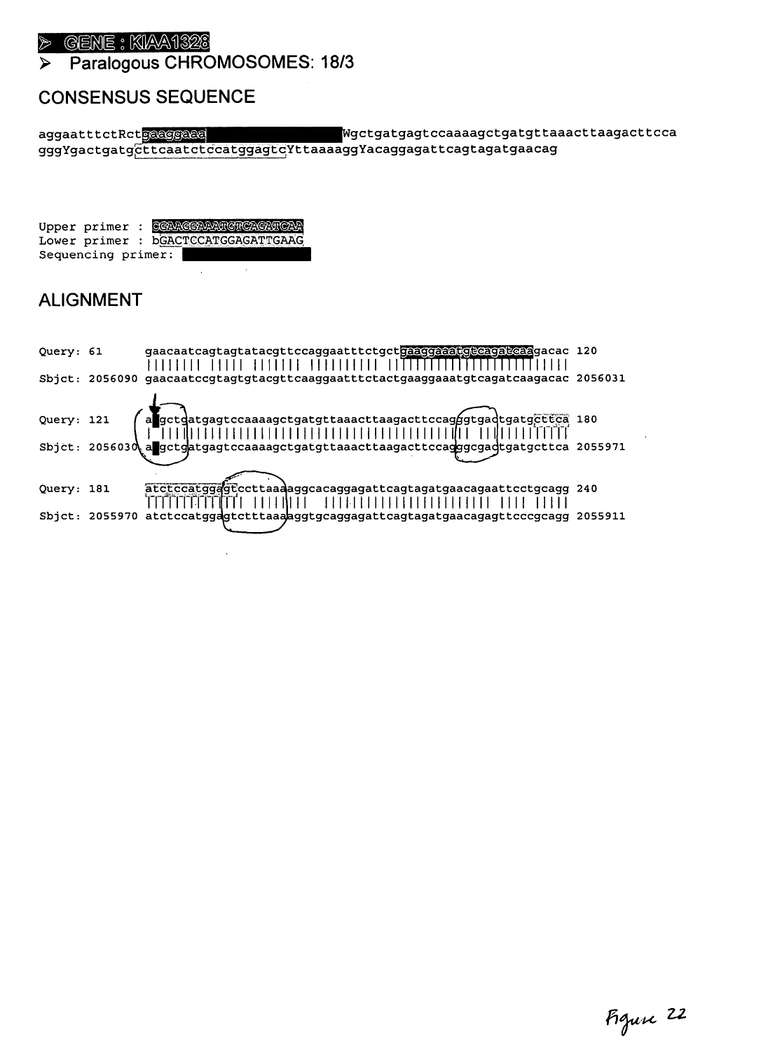

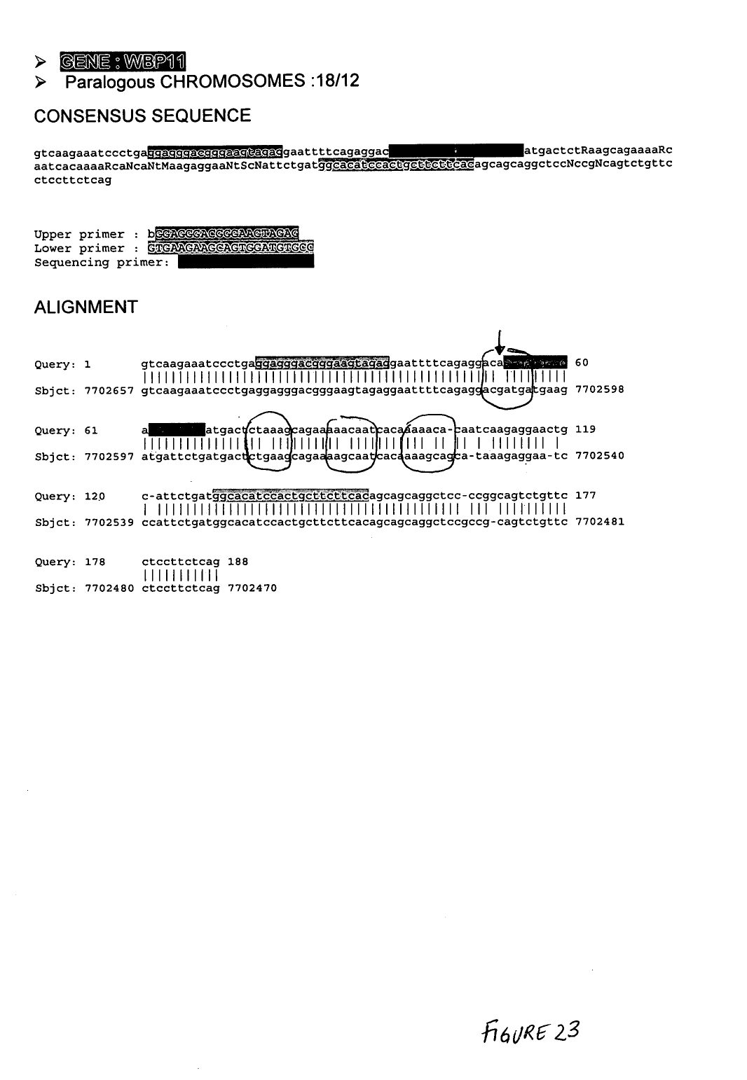

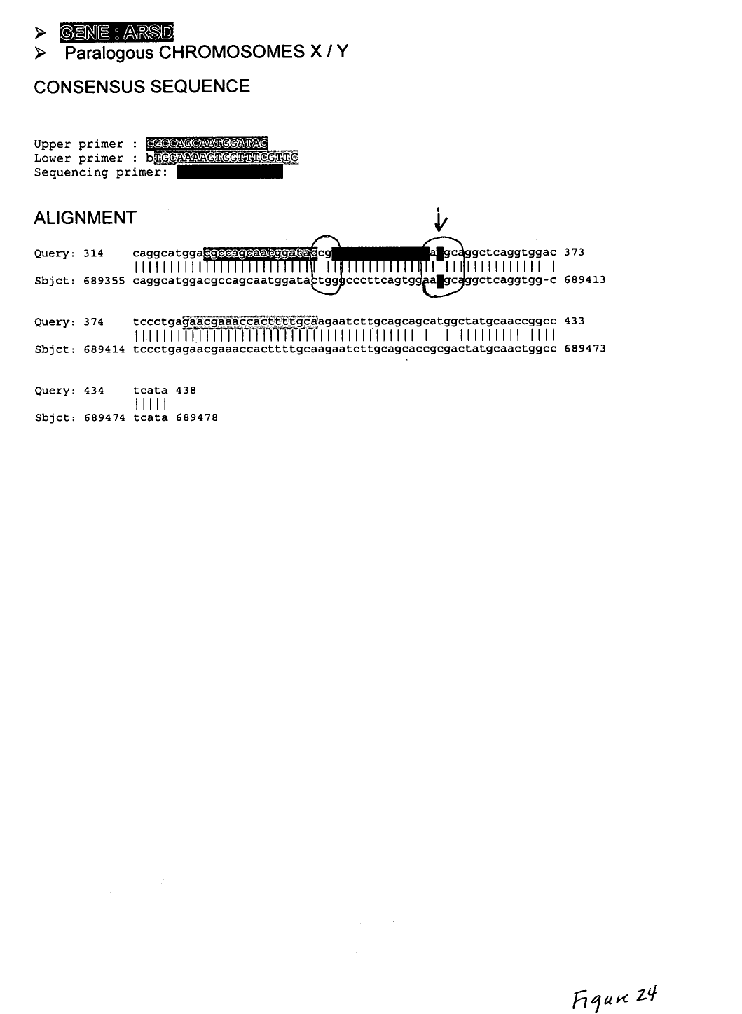

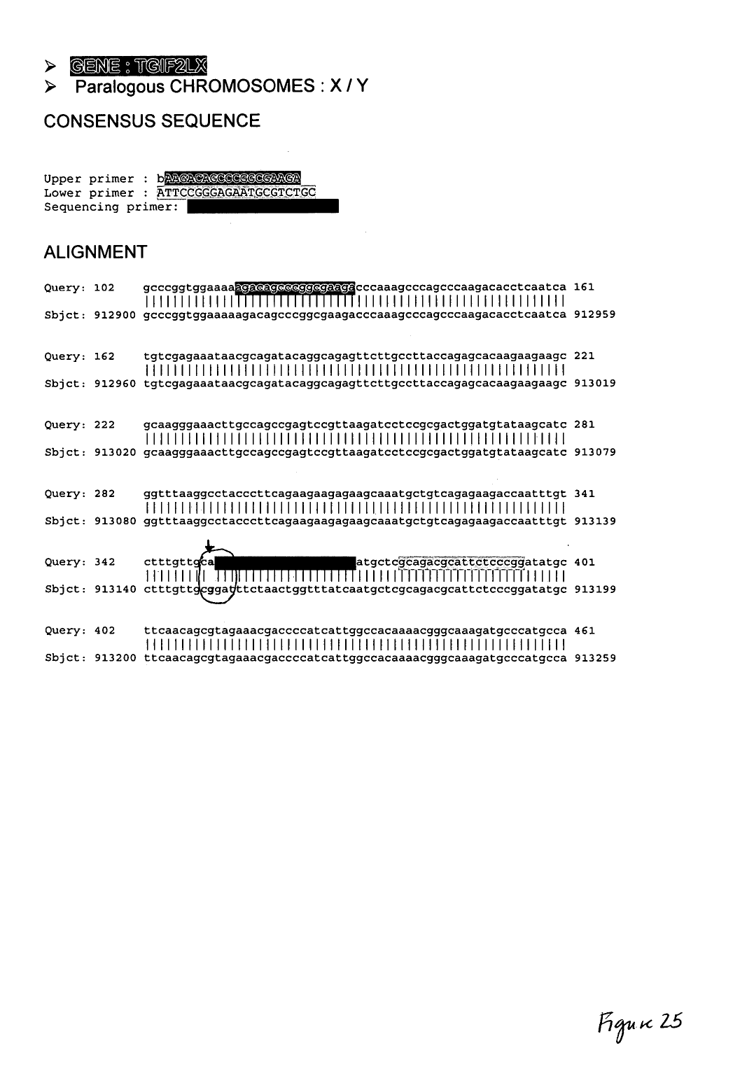

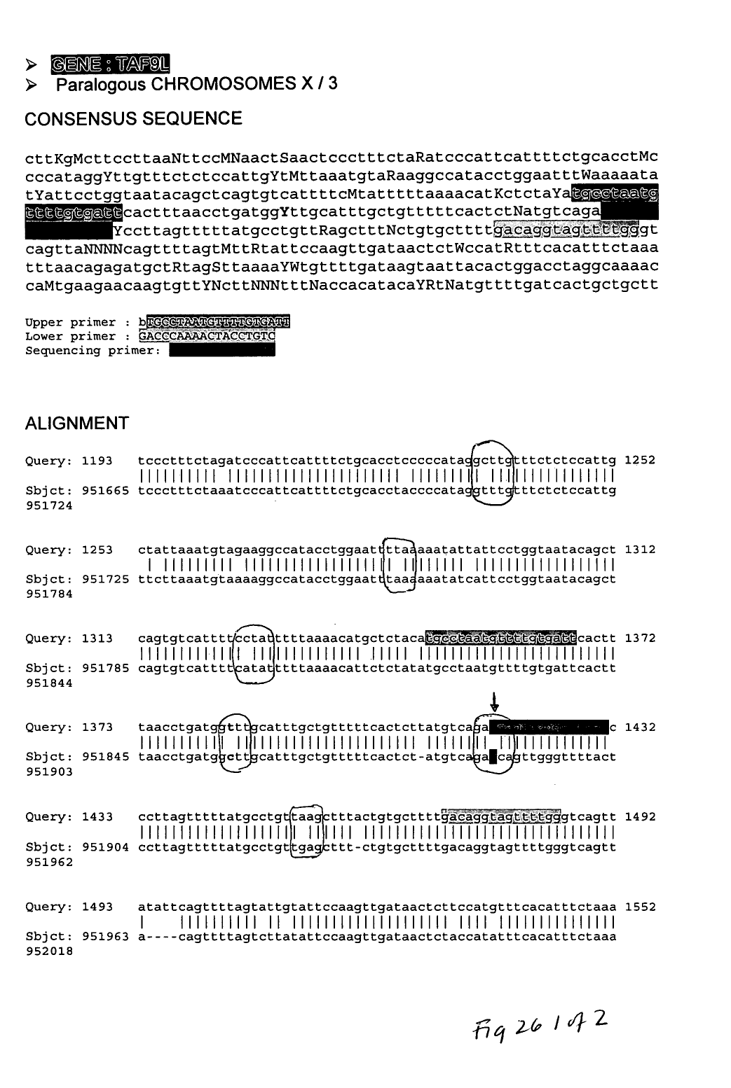

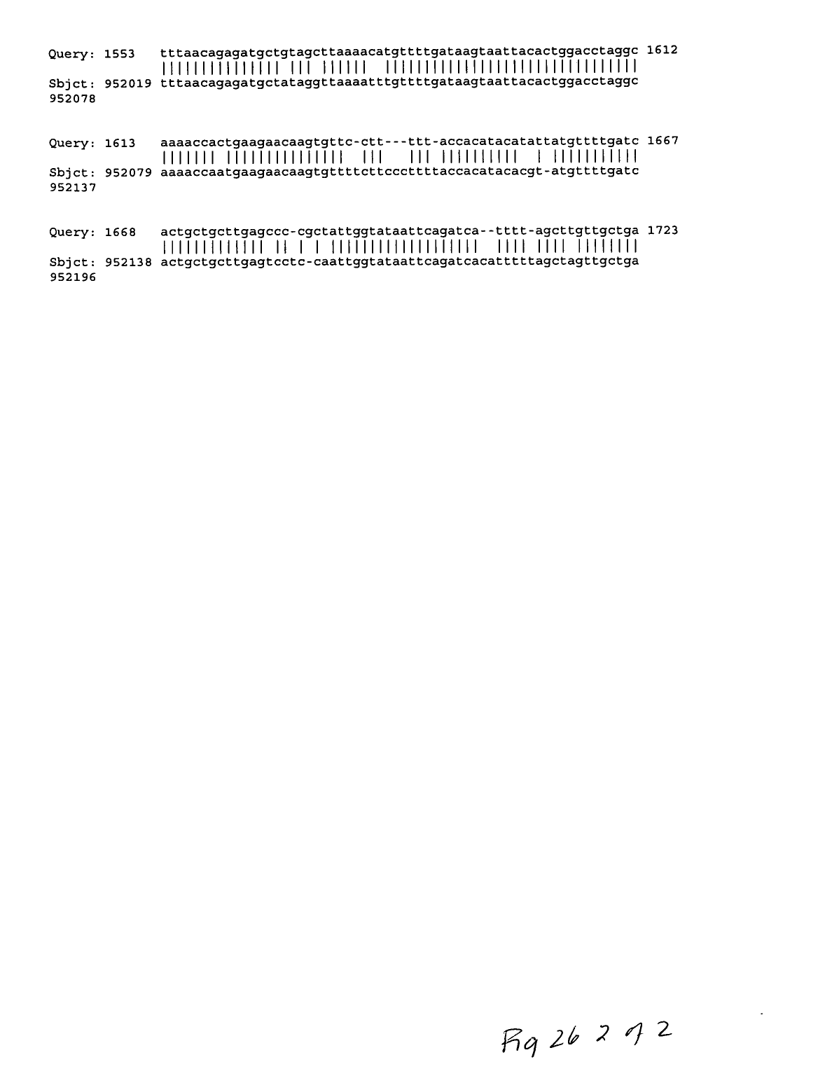

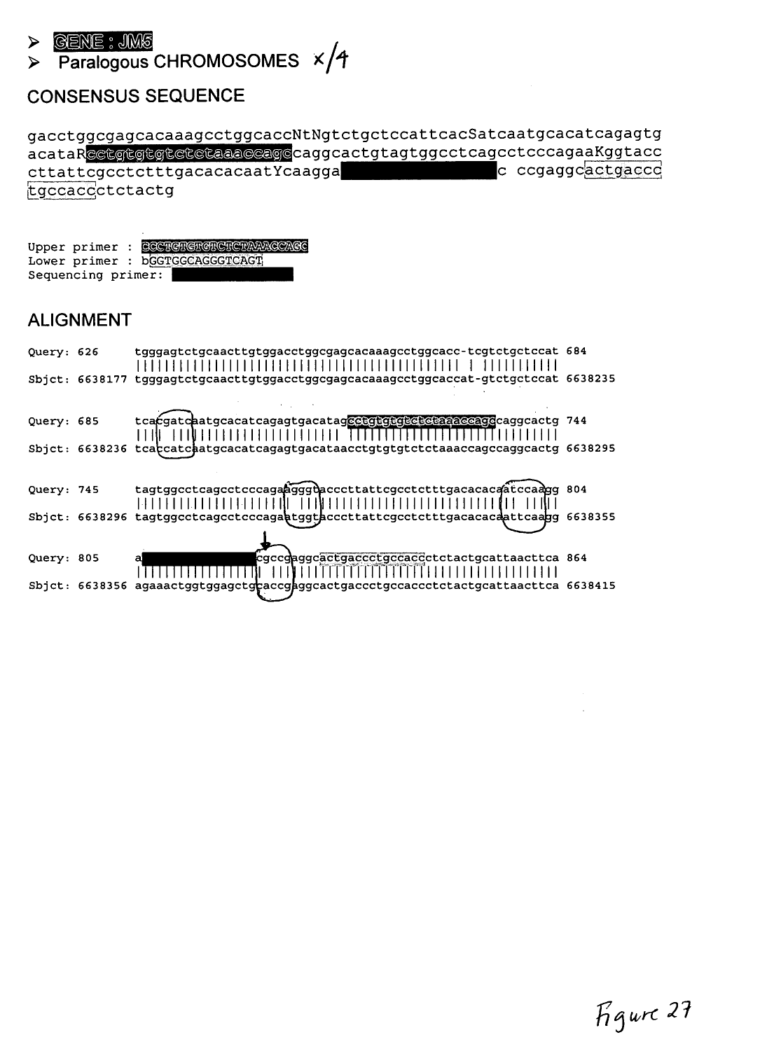

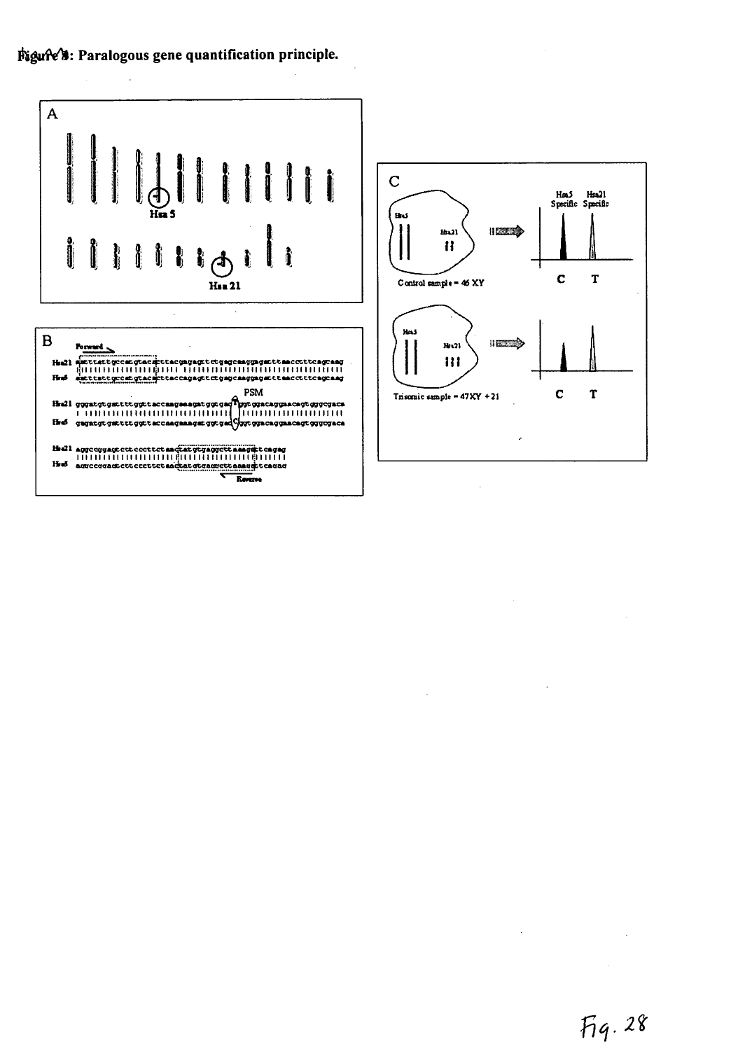

41. The method of claim 40, wherein said NUFIP1 paralogue comprises the sequence presented in FIG. 20.

42. The method according to claim 3, wherein said pair of primers comprises NUFIP1F (GCTGAGCCGACTAGTGATT, SEQ ID NO 9) and NUFIP1R (AAGGGAAGCGAGGACGTAA, SEQ ID NO 10).

43. The method according to claim 1 wherein said first sequence comprises the sequence of an STK24F paralogue and the second sequence comprises the sequence of STK24.

44. The method of claim 43, wherein said STK24R paralogue comprises the sequence presented in FIG. 21.

45. The method according to claim 3, wherein said pair of primers comprises STK24F (CGCTCTCGTCTGACATTT, SEQ ID NO 11) and STK24R (TCAGACATTTTTAGGTGG, SEQ ID NO 12).

46. The method according to claim 1 wherein said first sequence comprises the sequence of a KIAA1328 paralogue and the second sequence comprises the sequence of KIAA1328.

47. The method of claim 46, wherein said KIAA1328 paralogue comprises the sequence presented in FIG. 22.

48. The method according to claim 3, wherein said pair of primers comprises KIAA1328F (CGAAGGAAATGTCAGATCAA, SEQ ID NO 13) and KIAA1328R (GACTCCATGGAGATTGAAG, SEQ ID NO 14).

49. The method according to claim 1 wherein said first sequence comprises the sequence of a WBP11 paralogue and the second sequence comprises the sequence of WBP11.

50. The method of claim 49, wherein said WBP11 paralogue comprises the sequence presented in FIG. 23.

51. The method according to claim 3, wherein said pair of primers comprises WBP11F (GGAGGGACGGGAAGTAGAG, SEQ ID NO 15) and WBP11R (GTGAAGAAGCAGTGGATGTGCC SEQ ID NO 16).

52. The method according to claim 1 wherein said first sequence comprises the sequence of an ARSD paralogue and the second sequence comprises the sequence of ARSD.

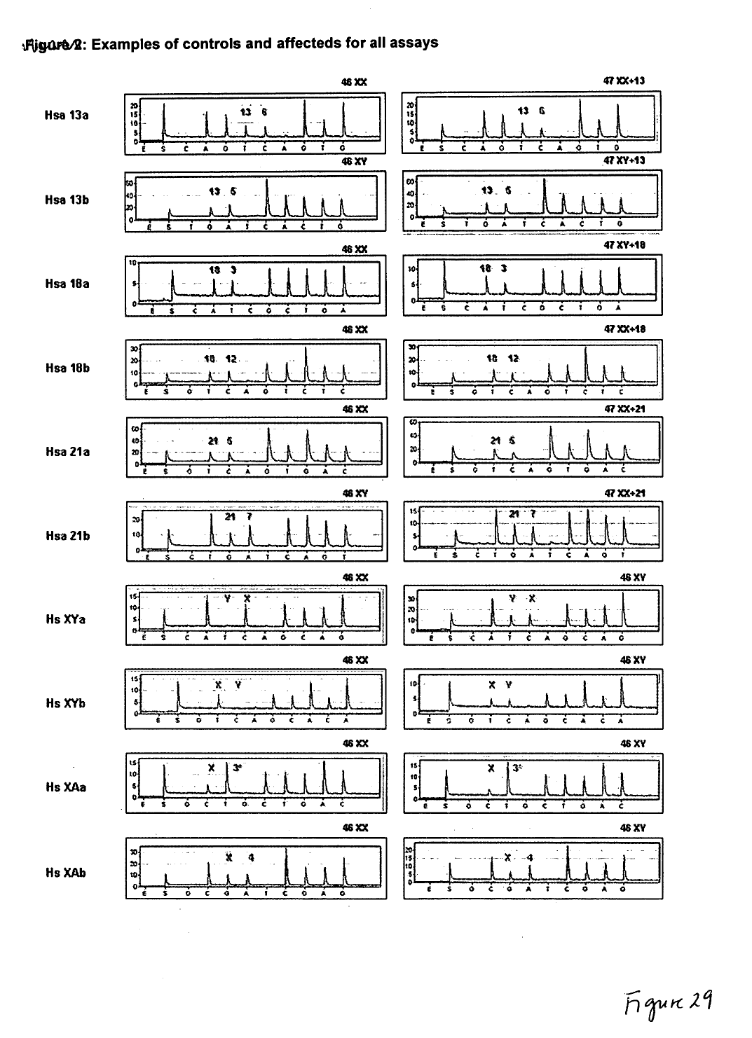

53. The method of claim 52, wherein said ARSDD paralogue comprises the sequence presented in FIG. 24.

54. The method according to claim 3, wherein said pair of primers comprises ARSDF (CGCCAGCAATGGATAC, SEQ ID NO 17) and ARSDR (TGCAAAAGTGGTTTCGTTC, SEQ ID NO 18).

55. The method according to claim 1 wherein said first sequence comprises the sequence of a TGIF2LX paralogue and the second sequence comprises the sequence of TGIF2LX.

56. The method of claim 55, wherein said TGIF2LX paralogue comprises the sequence presented in FIG. 25.

57. The method according to claim 3, wherein said pair of primers comprises TGIF2LXF (AAGACAGCCCGGCGAAGA, SEQ ID NO 19) and TGIF2LXR (ATTCCGGGAGAATGCGTCTGC, SEQ ID NO 20).

58. The method according to claim 1 wherein said first sequence comprises the sequence of a TAF9L paralogue and the second sequence comprises the sequence of TAF9L.

59. The method of claim 58, wherein said TAF9L paralogue comprises the sequence presented in FIG. 26.

60. The method according to claim 3, wherein said pair of primers comprises TAF9LF (TGCCTAATGTTTTGTGATT, SEQ ID NO 21) and TA9LR (GACCCAAAACTACCTGTC, SEQ ID NO 22).

61. The method according to claim 1 wherein said first sequence comprises the sequence of a JM5 paralogue and the second sequence comprises the sequence of JM5.

62. The method of claim 61, wherein said JM5 paralogue comprises the sequence presented in FIG. 27.

63. The method according to claim 3, wherein said pair of primers comprises JM5F (CCCTGTGTGTCTCTAAACCAGC, SEQ ID NO 23) and JM5R (GGTGGCAGGGTCAGT, SEQ ID NO 24).

64. The method according to claim 24, wherein said CCT8 paralogue comprises the sequence presented in FIG. 4.
